SEQUENCE LISTING (19 sequences)

SEQ ID NO: 1 - DNA, 4841 bp, synthetic construct
  source 1..4841: mol_type = other DNA
ttcgaaacgcgctagtgtagaggatctgtgcagttcaacctgttgatagcatgcaggcgggctcacctccgtgtgggatatcgtacgggcaacccgagaaggctgagcctggactcaacgggttgctgggtggactccagactcggggcgacgactcttcacgcgcagagcaagggcgtcgagcggtcgtgaaagtcttagtaccgcacgtgccgactcactggggatattgcctggagctgtaccgttctaggggggggaggttggagacctcctcttctcacgactggacccgcgagggccgcgttgccggttcccccagaggctgaagaacaagggcttactgtgggcagggggacgcccattcagcggctggcgctttacgcgtccaatcctgtccctagtggccccctgccgcggaagaacttttctatttctgggtaggcatcatcaggagcaggaaaactagggataacagggtaattaggctgagctcaattctgtcagccgttaagtgttcctgtgtcactcaaaattgctttgagaggctctaagggcttctcagtgcgttacatccctggcttgttgtccacaaccgttaaaccttaaaagctttaaaagccttatatattctttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacactagaagatgcatggacgcaccgctagcagcgcccctagcggtatcctataaaaaaacacaccgcgccgctagcagcacccctaatataaaataatgttttttataaaaatagtcagtaccacccctacaaaacggtgtcggcgcgttgttgtagccgcgccgacaccgcttttttaaatatcataaagagagtaagagaaactaatttttcataacactctatttataaagaaaaatcagcaaaaacttgtttttgcgtggggtgtggtgcttttggtggtgagaaccaccaacctgttgagcctttttgtggaactagtactcgaattcgttgacagctagctcagtcctaggtataatgctagctaggggccactagggacaggatgttttagagctagaaatagcaagttaaaataaggctagtccgttatcaacttgaaaaagtggcaccgagtcggtgctttttttctagatagtggatcctagggataacagggtaatgtttgccctctctcttcattcagggtcatgagaggcacgccattcaaggggagaagtgagatcggtaccggggccactagggacaggattggcaattgggcgcgccctcattgtagtttctgcggccgcccacacggaggtgagcccgcctattagctagcgtcgactctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatggccgcggtaccagatctttaaatctagacggtgaccacaacgcgcccgctttgatcggggacgtctgcggccgaccatttacgggtcttgttgtcgttggcggtcatgggccgaacatactcacccggatcggagggccgaggacaaggtcgaacgaggggcatgacccggtgcggggcttcttgcactcggcataggcgagtgctaagaataacgttggcactcgcgaccggttgttgacaattaatcatcggcatagtatatcggcatagtataatacgacaaggtgaggaactaaacccaggaggcagatcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcgggactctgggg

SEQ ID NO: 2 - DNA, 10681 bp, synthetic construct
  source 1..10681: mol_type = other DNA
atgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgttcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatacccatgggctagcctcgagtctagacctaggcgtacgtcgatgcaggtgtctagattaagaaataatcttcatctaaaatatacttcagtcacctcctagctgactcaaatcaatgcgtgtttcataaagaccagtgatggattgatggataagagtggcatctaaaacttcttttgtagacgtatatcgtttacgatcaattgttgtatcaaaatatttaaaagcagcgggagctccaagattcgtcaacgtaaataaatgaataatattttctgcttgttcacgtattggtttgtctctatgtttgttatatgcactaagaactttatctaaattggcatctgctaaaataacacgcttagaaaattcactgatttgctcaataatctcatctaaataatgcttatgctgctccacaaacaattgtttttgttcgttatcttctggactacccttcaacttttcataatgactagctaaatataaaaaattcacatatttgcttggcagagccagctcatttcctttttgtaattctccggcactagccagcatccgtttacgaccgttttctaactcaaaaagactatatttaggtagtttaatgattaagtcttttttaacttccttatatcctttagcttctaaaaagtcaatcggatttttttcaaaggaacttctttccataattgtgatccctagtaactctttaacggattttaacttcttcgatttccctttttccaccttagcaaccactaggactgaataagctaccgttggactatcaaaaccaccatatttttttggatcccagtcttttttacgagcaataagcttgtccgaatttctttttggtaaaattgactccttggagaatccgcctgtctgtacttctgttttcttgacaatattgacttggggcatggacaatactttgcgcactgtggcaaaatctcgccctttatcccagacaatttctccagtttccccattagtttcgattagagggcgtttgcgaatctctccatttgcaagtgtaatttctgttttgaagaagttcatgatattagagtaaaagaaatattttgcggttgctttgcctatttcttgctcagacttagcaatcattttacgaacatcataaactttataatcaccatagacaaactccgattcaagttttggatatttcttaatcaaagcagttccaacgacggcatttagatacgcatcatgggcatgatggtaattgttaatctcacgtactttatagaattggaaatcttttcggaagtcagaaactaatttagattttaaggtaatcactttaacctctcgaataagtttatcattttcatcgtatttagtattcatgcgactatccaaaatttgtgccacatgcttagtgatttggcgagtttcaaccaattggcgtttgataaaaccagctttatcaagttcactcaaacctccacgttcagctttcgttaaattatcaaacttacgttgagtgattaacttggcgtttagaagttgtctccaatagtttttcatctttttgactacttcttcacttggaacgttatccgatttaccacgatttttatcagaacgcgttaagaccttattgtctattgaatcgtctttaaggaaactttgtggaacaatgtgatcgacatcataatcacttaaacgattaatatctaattcttggtccacatacatgtctcttccattttggagataatagagatagagcttttcattttgcaattgagtattttcaacaggatgctctttaagaatctgacttcctaattctttgataccttcttcgattcgtttcatacgctctcgcgaatttttctggcccttttgagttgtctgattttcacgtgccatttcaataacgatattttctggcttatgccgccccattactttgaccaattcatcaacaacttttacagtctgtaaaataccttttttaatagcagggctaccagctaaatttgcaatatgttcatgtaaactatcgccttgtccagacacttgtgctttttgaatgtcttctttaaatgtcaaactatcatcatggatcagctgcataaaattgcgattggcaaaaccatctgatttcaaaaaatctaatattgttttgccagattgcttatccctaataccattaatcaattttcgagacaaacgtccccaaccagtataacggcgacgtttaagctgtttcatcaccttatcatcaaagaggtgagcatatgttttaagtctttcctcaatcatctccctatcttcaaataaggtcaatgttaaaacaatatcctctaagatatcttcattttcttcattatccaaaaaatctttatctttaataatttttagcaaatcatggtaggtacctaatgaagcattaaatctatcttcaactcctgaaatttcaacactatcaaaacattctatttttttgaaataatcttcttttaattgcttaacggttacttttcgatttgttttgaagagtaaatcaacaatggctttcttctgttcacctgaaagaaatgctggttttcgcattccttcagtaacatatttgacctttgtcaattcgttataaaccgtaaaatactcataaagcaaactatgttttggtagtactttttcatttggaagatttttatcaaagtttgtcatgcgttcaataaatgattgagctgaagcacctttatcgacaacttcttcaaaattccatggggtaattgtttcttcagacttccgagtcatccatgcaaaacgactattgccacgcgccaatggaccaacataataaggaattcgaaaagtcaagattttttcaatcttctcacgattgtcttttaaaaatggataaaagtcttcttgtcttctcaaaatagcatgcagctcacccaagtgaatttgatggggaatagagccgttgtcaaaggtccgttgcttgcgcagcaaatcttcacgatttagtttcaccaataattcctcagtaccatccattttttctaaaattggtttgataaatttataaaattcttcttggctagctcccccatcaatataacctgcatatccgttttttgattgatcaaaaaagatttctttatacttttctggaagttgttgtcgaactaaagcttttaaaagagtcaagtcttgatgatgttcatcgtagcgtttaatcattgaagctgataggggagccttagttatttcagtatttactcttaggatatctgaaagtaaaatagcatctgataaattcttagctgccaaaaacaaatcagcatattgatctccaatttgcgccaataaattatctaaatcatcatcgtaagtatcttttgaaagctgtaatttagcatcttctgccaaatcaaaatttgatttaaaattaggggtcaaacccaatgacaaagcaatgagattcccaaataagccatttttcttctcaccggggagctgagcaatgagattttctaatcgtcttgatttactcaatcgtgcagaaagaatcgctttagcatctactccacttgcgttaatagggttttcttcaaataattgattgtaggtttgtaccaactggataaatagtttgtccacatcactattatcaggatttaaatctccctcaatcaaaaaatgaccacgaaacttaatcatatgcgctaaggccaaatagattaagcgcaaatccgctttatcagtagaatctaccaatttttttcgcagatgatagatagttggatatttctcatgataagcaacttcatctactatatttccaaaaataggatgacgttcatgcttcttgtcttcttccaccaaaaaagactcttcaagtcgatgaaagaaactatcatctactttcgccatctcatttgaaaaaatctcctgtagataacaaatacgattcttccgacgtgtataccttctacgagctgtccgtttgagacgagtcgcttccgctgtctctccactgtcaaataaaagagcccctataagattttttttgatactgtggcggtctgtatttcccagaaccttgaactttttagacggaaccttatattcatcagtgatcaccgcccatccgacgctatttgtgccgatatctaagcctattgagtatttcttatccattttttataacctccttagagctcgaattcccaaaaaaacgggtatggagaaacagtagagagttgcgataaaaagcgtcaggtaggatccgctaatcttatggataaaaatgctatggcatagcaaagtgtgacgccgtgcaaataatcaatgtggacttttctgccgtgattatagacacttttgttacgcgtttttgtcatggctttggtcccgctttgttacagaatgcttttaataagcggggttaccggtttggttagcgagaagagccagtaaaagacgcagtgacggcaatgtctgatgcaatatggacaattggtttcttctctgaatggcgggagtatgaaaagtatggctgaagcgcaaaatgatcccctgctgccgggatactcgtttaatgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtttgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacggggttctttcgcccggatgaagcgcaccagccgcatttcagcgacctgtttgggcaaatcattaacgccgggcaaggggaagggcgctattcggagctgctggcgataaatctgcttgagcaattgttactgcggcacatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggttgtgaagaaaaagtgaatgatgtagccgtcaagttgtcataattggtaacgaatcagacaattgacggcttgacggagtagcatagggtttgcagaatccctgcttcgtccatttgacaggcacattatgcatttaattaatggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaaattcagcaggatcaccatatggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataaaacgaatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaacaatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgacaccggacattatcctgcagcgtaccgggatcgatgtgagagctgtcgaacagggggaagatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatgacgcatcctcacgataatatccgggtaggcgcaatcactttcgtctctctagggaattcagcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgattgcggccgcaaccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgt

SEQ ID NO: 3 - DNA, 3696 bp, synthetic construct
  source 1..3696: mol_type = other DNA
gcggccaattcgatctagaattccattgagtaagtttttaagcacatcagcttcaaaagcctgcgagggctagaattacctaccggcctccaccatgcctgcggcatgcccaatcctgtccctagtggccccgctagccccatcttaattaagcgataagcttgatatcgaattcccccgggaccaaaatggcaagtaaaatagcctgatgggataggctctaagtccaacgaaccagtgtcaccactgacacaatgaggaaaaccatgtcacatctcgcagaactggttgccagtgcgaaggcggccattagccaggcgtcagatgttgccgcgttagataatgtgcgcgtcgaatatttgggtaaaaaagggcacttaacccttcagatgacgaccctgcgtgagctgccgccagaagagcgtccggcagctggtgcggttatcaacgaagcgaaagagcaggttcagcaggcgctgaatgcgcgtaaagcggaactggaaagcgctgcactgaatgcgcgtctggcggcggaaacgattgatgtctctctgccaggtcgtcgcattgaaaacggcggtctgcatccggttacccgtaccatcgaccgtatcgaaagtttcttcggtgagcttggctttaccgtggcaaccgggccggaaatcgaagacgattatcataacttcgatgctctgaacattcctggtcaccacccggcgcgcgctgaccacgacactttctggtttgacactacccgcctgctgcgtacccagacctctggcgtacagatccgcaccatgaaagcccagcagccaccgattcgtatcatcgcgcctggccgtgtttatcgtaacgactacgaccagactcacacgccgatgttccatcagatggaaggtctgattgttgataccaacatcagctttaccaacctgaaaggcacgctgcacgacttcctgcgtaacttctttgaggaagatttgcagattcgcttccgtccttcctacttcccgtttgcggaaccttctgcagaagtggacgtcatgggtaaaaacggtaaatggctggaagtgctgggctgcgggatggtgcatccgaacgtgttgcgtaacgttggcatcgacccggaagtttactctggtttcggcttcgggatggggatggagcgtctgactatgttgcgttacggcgtcaccgacctgcgttcattcttcgaaaacgatctgcgtttcctcaaacagtttaaataaggcaggaatagattatgaaattcagtgaactgtggttacgcgaatgggtgaacccggcgattgatagcgatgcgctggcaaatcaaatcactatggcgggcctggaagttgggggatctggcgcgccggggccactagggacaggattggcaattgtacatcgtacgggcaacccgagaaggctgagcctggactcaacgggttgctgggtggactccagactcggggcgacgactcttcacgcgcagagcaagggcgtcgagcggtcgtgaaagtcttagtaccgcacgtgccgactcactggggatattgcctggagctgtaccgttctaggggggggaggttggagacctcctcttctcacgactggacccgcgagggccgcgttgccggttcccccagaggctgaagaacaagggcttactgtgggcagggggacgcccattcagcggctggcgctttgatgtaggcttcatgaaagcttggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttatagtccggaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgattgcggcagtttttcgggtggtttgttgccatttttacctgtctgctgccgtgatcgcgctgaacgcgttttagcggtgcgtacaattaagggattatggtaaatccacttactgtctgccctcgtagccatcggtcatgacctttgtcaacagcaatggatcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccnnnnnnnnnnnnnnnnnnnn

SEQ ID NO: 4 - DNA, 4881 bp, synthetic construct
  source 1..4881: mol_type = other DNA
ttcgaaacgcgctagtgtagaggatctgtgcagttcaacctgttgatagcatgcaggcgggctcacctccgtgtgggatatcgtacgggcaacccgagaaggctgagcctggactcaacgggttgctgggtggactccagactcggggcgacgactcttcacgcgcagagcaagggcgtcgagcggtcgtgaaagtcttagtaccgcacgtgccgactcactggggatattgcctggagctgtaccgttctaggggggggaggttggagacctcctcttctcacgactggacccgcgagggccgcgttgccggttcccccagaggctgaagaacaagggcttactgtgggcagggggacgcccattcagcggctggcgctttacgcgtccaatcctgtccctagtggccccctgccgcggaagaacttttctatttctgggtaggcatcatcaggagcaggaaaactagggataacagggtaattaggctgagctcaattctgtcagccgttaagtgttcctgtgtcactcaaaattgctttgagaggctctaagggcttctcagtgcgttacatccctggcttgttgtccacaaccgttaaaccttaaaagctttaaaagccttatatattctttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacactagaagatgcatggacgcaccgctagcagcgcccctagcggtatcctataaaaaaacacaccgcgccgctagcagcacccctaatataaaataatgttttttataaaaatagtcagtaccacccctacaaaacggtgtcggcgcgttgttgtagccgcgccgacaccgcttttttaaatatcataaagagagtaagagaaactaatttttcataacactctatttataaagaaaaatcagcaaaaacttgtttttgcgtggggtgtggtgcttttggtggtgagaaccaccaacctgttgagcctttttgtggaactagtactcgaattcgttgacagctagctcagtcctaggtataatgctagctaggggccactagggacaggatgttttagagctagaaatagcaagttaaaataaggctagtccgttatcaacttgaaaaagtggcaccgagtcggtgctttttttctagatagtggatcctagggataacagggtaatgtttgccctctctcttcattcagggtcatgagaggcacgccattcaaggggagaagtgagatcggtaccggggccactagggacaggattggcaattgcgagggctagaattacctaccggcctccaccatgcctgcgggcgcgccctcattgtagtttctgcggccgcccacacggaggtgagcccgcctattagctagcgtcgactctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatggccgcggtaccagatctttaaatctagacggtgaccacaacgcgcccgctttgatcggggacgtctgcggccgaccatttacgggtcttgttgtcgttggcggtcatgggccgaacatactcacccggatcggagggccgaggacaaggtcgaacgaggggcatgacccggtgcggggcttcttgcactcggcataggcgagtgctaagaataacgttggcactcgcgaccggttgttgacaattaatcatcggcatagtatatcggcatagtataatacgacaaggtgaggaactaaacccaggaggcagatcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcgggactctgggg

SEQ ID NO: 5 - DNA, 4043 bp, synthetic construct
  source 1..4043: mol_type = other DNA
agtggatcctagggataacagggtaatgtttgccctctctcttcattcagggtcatgagaggcacgccattcaaggggagaagtgagatcggtacccaggcgggctcacctccgtgtgggcggccatggcgcgccagctagtagcggccgcccaatcctgtccctagtggccccgcccctaggactagtacgcgcgataagcttgatatcgaattcccccgggaccaaaatggcaagtaaaatagcctgatgggataggctctaagtccaacgaaccagtgtcaccactgacacaatgaggaaaaccatgtcacatctcgcagaactggttgccagtgcgaaggcggccattagccaggcgtcagatgttgccgcgttagataatgtgcgcgtcgaatatttgggtaaaaaagggcacttaacccttcagatgacgaccctgcgtgagctgccgccagaagagcgtccggcagctggtgcggttatcaacgaagcgaaagagcaggttcagcaggcgctgaatgcgcgtaaagcggaactggaaagcgctgcactgaatgcgcgtctggcggcggaaacgattgatgtctctctgccaggtcgtcgcattgaaaacggcggtctgcatccggttacccgtaccatcgaccgtatcgaaagtttcttcggtgagcttggctttaccgtggcaaccgggccggaaatcgaagacgattatcataacttcgatgctctgaacattcctggtcaccacccggcgcgcgctgaccacgacactttctggtttgacactacccgcctgctgcgtacccagacctctggcgtacagatccgcaccatgaaagcccagcagccaccgattcgtatcatcgcgcctggccgtgtttatcgtaacgactacgaccagactcacacgccgatgttccatcagatggaaggtctgattgttgataccaacatcagctttaccaacctgaaaggcacgctgcacgacttcctgcgtaacttctttgaggaagatttgcagattcgcttccgtccttcctacttcccgtttgcggaaccttctgcagaagtggacgtcatgggtaaaaacggtaaatggctggaagtgctgggctgcgggatggtgcatccgaacgtgttgcgtaacgttggcatcgacccggaagtttactctggtttcggcttcgggatggggatggagcgtctgactatgttgcgttacggcgtcaccgacctgcgttcattctttgagaacgatctgcgtttcctcaaacagtttaaataaggcaggaatagattatgaaattcagtgaactgtggttacgcgaatgggtgaacccggcgattgatagcgatgcgctggcaaagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcaccgcatgcctcccagattatatcactcgacgacgtcaccggttcgcgattcgaaacccggggccactagggacaggattgggatatcgtacgggcaacccgagaaggctgagcctggactcaacgggttgctgggtggactccagactcggggcgacgactcttcacgcgcagagcaagggcgtcgagcggtcgtgaaagtcttagtaccgcacgtgccgactcactggggatattgcctggagctgtaccgttctaggggggggaggttggagacctcctcttctcacgactggacccgcgagggccgcgttgccggttcccccagaggctgaagaacaagggcttactgtgggcagggggacgcccattcagcggctggcgctttacgcgtccacacggaggtgagcccgcctgctgccgcggaagaacttttctatttctgggtaggcatcatcaggagcaggaaaactagggataacagggtaattaggctgagctcaattctgtcagccgttaagtgttcctgtgtcactcaaaattgctttgagaggctctaagggcttctcagtgcgttacatccctggcttgttgtccacaaccgttaaaccttaaaagctttaaaagccttatatattctttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacactagaagatgcatggacgcaccgctagcagcgcccctagcggtatcctataaaaaaacacaccgcgccgctagcagcacccctaatataaaataatgttttttataaaaatagtcagtaccacccctacaaaacggtgtcggcgcgttgttgtagccgcgccgacaccgcttttttaaatatcataaagagagtaagagaaactaatttttcataacactctatttataaagaaaaatcagcaaaaacttgtttttgcgtggggtgtggtgcttttggtggtgagaaccaccaacctgttgagcctttttgtggaactagtactcgaattcgttgacagctagctcagtcctaggtataatgctagctacaggcgggctcacctccgtggttttagagctagaaatagcaagttaaaataaggctagtccgttatcaacttgaaaaagtggcaccgagtcggtgctttttttctagat

SEQ ID NO: 6 - DNA, 3913 bp, synthetic construct
  source 1..3913: mol_type = other DNA
tctgaaggctcttctccttgagccacctgtccgccaaggcaaagcgctcacagcagtggtcattctcgagataatcgacgcgtaccaacttgccatcctgaagaatggtgcagtgtctcggcaccccatagggaacctttgccatcaactcggcaagatgcagcgtcgtgttggcatcgtgtcccacgccgaggagaagtacctgcccatcgagttcatggacacgggcgaccgggcttgcaggcgagtgaggtggcaggggcaatggatcagagatgatctgctctgcctgtggccccgctgccgcaaaggcaaatggatgggcgctgcgctttacatttggcaggcgccagaatgtgtcagagacaactccaaggtccggtgtaacgggcgacgtggcaggatcgaacggctcgtcgtccagacctgaccacgagggcatgacgagcgtccctcccggacccagcgcagcacgcagggcctcgatcagtccaagtggcccatcttcgaggggccggacgctacggaaggagctgtggaccagcagcacaccgccgggggtaaccccaaggttgagaagctgaccgatgagctcggcttttcgccattcgtattgcacgacattgcactccaccgctgatgacataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagctctagtagttacattgtcgatcgcatgcctcccagattatatcactcgacgacgtcaccggttcgcgattcgaaacccggggccactagggacaggattgggatatcgtacgggcaacccgagaaggctgagcctggactcaacgggttgctgggtggactccagactcggggcgacgactcttcacgcgcagagcaagggcgtcgagcggtcgtgaaagtcttagtaccgcacgtgccgactcactggggatattgcctggagctgtaccgttctaggggggggaggttggagacctcctcttctcacgactggacccgcgagggccgcgttgccggttcccccagaggctgaagaacaagggcttactgtgggcagggggacgcccattcagcggctggcgctttacgcgtccacacggaggtgagcccgcctgctgccgcggaagaacttttctatttctgggtaggcatcatcaggagcaggaaaactagggataacagggtaattaggctgagctcaattctgtcagccgttaagtgttcctgtgtcactcaaaattgctttgagaggctctaagggcttctcagtgcgttacatccctggcttgttgtccacaaccgttaaaccttaaaagctttaaaagccttatatattctttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacactagaagatgcatggacgcaccgctagcagcgcccctagcggtatcctataaaaaaacacaccgcgccgctagcagcacccctaatataaaataatgttttttataaaaatagtcagtaccacccctacaaaacggtgtcggcgcgttgttgtagccgcgccgacaccgcttttttaaatatcataaagagagtaagagaaactaatttttcataacactctatttataaagaaaaatcagcaaaaacttgtttttgcgtggggtgtggtgcttttggtggtgagaaccaccaacctgttgagcctttttgtggaactagtactcgaattcgttgacagctagctcagtcctaggtataatgctagctacaggcgggctcacctccgtggttttagagctagaaatagcaagttaaaataaggctagtccgttatcaacttgaaaaagtggcaccgagtcggtgctttttttctagatagtggatcctagggataacagggtaatgtttgccctctctcttcattcagggtcatgagaggcacgccattcaaggggagaagtgagatcggtacccaggcgggctcacctccgtgtgggcggccatggcgcgccagctagtagcggccgcccaatcctgtccctagtggccccgcccctaggactagtacgcgcgataagcttgatatcgaattcccccgggaccaaaatggcaagtaaaatagcctgatgggataggctctaagtccaacgaaccagtgtcaccactgacacaatgaggaaaaccatgtcacatctcgcagaactggttgccagtgcgaaggcggccattagccaggcgtcagatgttgccgcgttagataatgtgcgcgtcgaatatttgggtaaaaaagggcacttaacccttcagatgacgaccctgcgtgagctgccgccagaagagcgtccggcagctggtgcggttatcaacgaagcgaaagagcaggttcagcaggcgctgaatgcgcgtaaagcggaactggaaagcgctgcactgaatgcgcgtctggcggcggaaacgattgatgtctctctgccaggtcgtcgcattgaaaacggcggtctgcatccggttacccgtaccatcgaccgtatcgaaagtttcttcggtgagcttggctttaccgtggcaaccgggccggaaatcgaagacgattatcataacttcgatgctctgaacattcctggtcaccacccggcgcgcgctgaccacgacactttctggtttgacactacccgcctgctgcgtacccagacctctggcgtacagatccgcaccatgaaagcccagcagccaccgattcgtatcatcgcgcctggccgtgtttatcgtaacgactacgaccagactcacacgccgatgttccatcagatggaaggtctgattgttgataccaacatcagctttaccaacctgaaaggcacgctgcacgacttcctgcgtaacttctttgaggaagatttgcagattcgcttccgtccttcctacttcccgtttgcggaaccttctgcagaagtggacgtcatgggtaaaaacggtaaatggctggaagtgctgggctgcgggatggtgcatccgaacgtgttgcgtaacgttggcatcgacccggaagtttactctggtttcggcttcgggatggggatggagcgtctgactatgttgcgttacggcgtcaccgacctgcgttcattctttgagaacgatctgcgtttcctcaaacagtttaaataaggcaggaatagattatgaaattcagtgaactgtggttacgcgaatgggtgaacccggcgattgatagcgatgcgctggcaaatcaaatcactatggcgggcctggaagttgggggatctggaccaaaacgaaaaaagacgcttttcagcgtcttttttcgaaaatttggtaccgagcctcagccttcccttggggttatcattggggctcactcaaaggcggtaatcagataaaaaaaatccttagctttcgctaaggatgatttctgctttagccaatcgactggcgagcggcatcgcattcttcgcatcccgcctctggcggatgcaggaagatcaacggatctcggcccagttgacccagggctgtcgccacaatgtcgcgggagcggatcaaccgagcaaaggcatgaccgactggaccttcct

SEQ ID NO: 7 - protein, 473 aa, Bacillus subtilis
MNIKKFAKQATVLTFTTALLAGGATQAFAKETNQKPYKETYGISHITRHDMLQIPEQQKNEKYQVPEFDSSTIKNISSAKGLDVWDSWPLQNADGTVANYHGYHIVFALAGDPKNADDTSIYMFYQKVGETSIDSWKNAGRVFKDSDKFDANDSILKDQTQEWSGSATFTSDGKIRLFYTDFSGKHYGKQTLTTAQVNVSASDSSLNINGVEDYKSIFDGDGKTYQNVQQFIDEGNYSSGDNHTLRDPHYVEDKGHKYLVFEANTGTEDGYQGEESLFNKAYYGKSTSFFRQESQKLLQSDKKRTAELANGALGMIELNDDYTLKKVMKPLIASNTVTDEIERANVFKMNGKWYLFTDSRGSKMTIDGITSNDIYMLGYVSNSLTGPYKPLNKTGLVLKMDLDPNDVTFTYSHFAVPQAKGNNVVITSYMTNRGFYADKQSTFAPSFLLNIKGKKTSVVKDSILEQGQLTVNK

SEQ ID NO: 8 - protein, 341 aa, synthetic construct
MKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 9 - protein, 316 aa, synthetic construct
MSELVVFKANELAISRYDLTEHETKLILCCVALLNPTIENPTRKERTVSFTYNQYVQMMNISRENAYGVLAKATRELMTRTVEIRNPLVKGFEIFQWTNYAKFSSEKLELVFSEEILPYLFQLKKFIKYNLEHVKSFENKYSMRIYEWLLKELTQKKTHKANIEISLDEFKFMLMLENNYHEFKRLNQWVLKPISKDLNTYSNMKLVVDKRGRPTDTLIFQVELDRQMDLVTELENNQIKMNGDKIPTTITSDSYLHNGLRKTLHDALTAKIQLTSFEAKFLSDMQSKYDLNGSFSWLTQKQRTTLENILAKYGRI

SEQ ID NO: 10 - protein, 1368 aa, Streptococcus pyogenes [sequence omitted]

SEQ ID NO: 11 - protein, 292 aa, synthetic construct
MAEAQNDPLLPGYSFNAHLVAGLTPIEANGYLDFFIDRPLGMKGYILNLTIRGQGVVKNQGREFVCRPGDILLFPPGEIHHYGRHPEAREWYHQWVYFRPRAYWHEWLNWPSIFANTGFFRPDEAHQPHFSDLFGQIINAGQGEGRYSELLAINLLEQLLLRHMEAINESLHPPMDNRVREACQYISDHLADSNFDIASVAQHVCLSPSRLSHLFRQQLGISVLSWREDQRISQAKLLLSTTRMPIATVGRNVGFDDQLYFSRVFKKCTGASPSEFRAGCEEKVNDVAVKLS

SEQ ID NO: 12 - protein, 138 aa, synthetic construct
MDINTETEIKQKHSLTPFPVFLISPAFRGRYFHSYFRSSAMNAYYIQDRLEAQSWARHYQQLAREEKEAELADDMEKGLPQHLFESLCIDHLQRHGASKKSITRAFDDDVEFQERMAEHIRYMVETIAHHQVDIDSEV

SEQ ID NO: 13 - protein, 261 aa, synthetic construct
MSTALATLAGKLAERVGMDSVDPQELITTLRQTAFKGDASDAQFIALLIVANQYGLNPWTKEIYAFPDKQNGIVPVVGVDGWSRIINENQQFDGMDFEQDNESCTCRIYRKDRNHPICVTEWMDECRREPFKTREGREITGPWQSHPKRMLRHKAMIQCARLAFGFAGIYDKDEAERIVENTAYTAERQPERDITPVNDETMQEINTLLIALDKTWDDDLLPLCSQIFRRDIRASSELTQAEAVKALGFLKQKAAEQKVAA

SEQ ID NO: 14 - protein, 226 aa, synthetic construct
MTPDIILQRTGIDVRAVEQGEDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 15 - protein, 286 aa, synthetic construct
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 16 - protein, 327 aa, synthetic construct
MSHLAELVASAKAAISQASDVAALDNVRVEYLGKKGHLTLQMTTLRELPPEERPAAGAVINEAKEQVQQALNARKAELESAALNARLAAETIDVSLPGRRIENGGLHPVTRTIDRIESFFGELGFTVATGPEIEDDYHNFDALNIPGHHPARADHDTFWFDTTRLLRTQTSGVQIRTMKAQQPPIRIIAPGRVYRNDYDQTHTPMFHQMEGLIVDTNISFTNLKGTLHDFLRNFFEEDLQIRFRPSYFPFAEPSAEVDVMGKNGKWLEVLGCGMVHPNVLRNVGIDPEVYSGFGFGMGMERLTMLRYGVTDLRSFFENDLRFLKQFK

SEQ ID NO: 17 - protein, 177 aa, synthetic construct [sequence omitted]

SEQ ID NO: 18 - protein, 263 aa, synthetic construct
MREAVIAEVSTQLSEVVGVIERHLEPTLLAVHLYGSAVDGGLKPHSDIDLLVTVTVRLDETTRRALINDLLETSASPGESEILRAVEVTIVVHDDIIPWRYPAKRELQFGEWQRNDILAGIFEPATIDIDLAILLTKAREHSVALVGPAAEELFDPVPEQDLFEALNETLTLWNSPPDWAGDERNVVLTLSRIWYSAVTGKIAPKDVAADWAMERLPAQYQPVILEARQAYLGQEEDRLASRADQLEEFVHYVKGEITKVVGK

SEQ ID NO: 19 - protein, 267 aa, synthetic construct
MSSAVECNVVQYEWRKAELIGQLLNLGVTPGGVLLVHSSFRSVRPLEDGPLGLIEALRAALGPGGTLVMPSWSGLDDEPFDPATSPVTPDLGVVSDTFWRLPNVKRSAHPFAFAAAGPQAEQIISDPLPLPPHSPASPVARVHELDGQVLLLGVGHDANTTLHLAELMAKVPYGVPRHCTILQDGKLVRVDYLENDHCCERFALADRWLKEKSLQKEGPVGHAFARLIRSRDIVATALGQLGRDPLIFLHPPEAGCEECDAARQSIG

(19) United States
(12) Patent Application Publication
(10) Pub. No.: US 2026/0043048 A1
(43) Pub. Date: Feb. 12, 2026

(54) HIGH-THROUGHPUT IN VIVO DNA RECOMBINEERING

(71) Applicant: BacStitch DNA, Inc., South San Francisco, CA (US)

(72) Inventors: Sasha LEVY, South San Francisco, CA (US); Takeshi MATSUI, South San Francisco, CA (US)

(21) Appl. No.: 19/297,960

(22) Filed: Aug. 12, 2025

Related U.S. Application Data
(60) Provisional application No. 63/682,315, filed on Aug. 12, 2024.

(51) Int. Cl. C12N 15/90; C12N 9/22; C12N 15/11

(52) U.S. Cl. CPC C12N 15/902; C12N 9/226; C12N 15/111; C12N 2310/20

(57) ABSTRACT

Provided herein are methods and compositions for truncating and, optionally, joining polynucleotides. The methods bypass inefficient cloning methods and the requirement for expensive enzymes by using in vivo homologous recombination in conjunction with specific endonuclease digestion. The methods may further be used to assemble long fragments of DNA from existing DNA constructs or libraries or to generate novel DNA libraries.

### CROSS-REFERENCE TO RELATED APPLICATIONS

The present application claims priority to U.S. Provisional Patent Application No. 63/682,315 that was filed Aug. 12, 2024, the entire contents of which are hereby incorporated by reference.

### REFERENCE TO A SEQUENCE LISTING

This application is being filed electronically via Patent Center and includes an electronically submitted Sequence Listing in .txt format. The .txt file contains a sequence listing entitled “185383_00005.xml” created on Aug. 12, 2025, and is 54,012 bytes in size. The Sequence Listing contained in this .xml file is part of the specification and is hereby incorporated by reference herein in its entirety.

### BACKGROUND

Recent advances in recombinant oligonucleotide technology have ignited research in the fields of traditional biology and bioengineering. However, oligonucleotide assembly processes can be expensive and time-consuming, with requirements for multiple purification steps and various enzymes. Moreover, current molecular biology methods have limitations in the size and composition of DNA elements that can be combined. Thus, methods for assembling DNA elements (e.g. promoters, gene fragments, etc.) together are needed to address these limitations and bypass the necessity for numerous and expensive enzymes (e.g. ligases, etc.). New methods are required for efficient, high throughput, and versatile assembly of DNA fragments on a massively parallel scale.

Provided herein, inter alia, are solutions to these and other problems in the art.

### BRIEF SUMMARY OF THE INVENTION

In an aspect of the current disclosure, methods of truncating and, optionally, replacing, at least one DNA sequence are provided. In some embodiments, the methods comprise: (a) conjugating a first donor cell comprising a first donor construct with a first recipient cell comprising a first recipient polynucleotide to (i) transfer the first donor construct from the first donor cell to the first recipient cell and (ii) recombine the first donor construct and the first recipient construct in the first recipient cell by homologous recombination. The first donor construct comprises, from 5′ to 3′, (1) a first endonuclease site (C1), (2) a first homologous recombination region (HR1), optionally, a first joining polynucleotide sequence, which optionally comprises a seventh homologous recombination region (HR7), (3) a first selection cassette comprising at least one first selectable marker, (4) a second homologous recombination region (HR2), and (5) a second endonuclease site (C2). The recipient construct comprises, from 5′ to 3′, (1) a first recipient polynucleotide comprising a first sequence to be truncated comprising a third homologous recombination region (HR3) that is homologous to HR1, and (2) a fourth homologous recombination region (HR4) that is homologous to R2, and, optionally, further comprising a removable selectable construct comprising from 5′ to 3′ a third endonuclease site (C3), a second selection cassette comprising at least one second selectable marker, wherein the at least one second selectable marker is distinct from the at least one first selectable marker in the first selection cassette, and a fourth endonuclease site (C4), wherein HR3 ends at least one base pair (bp) upstream of the 3′ end of the first sequence to be truncated. Following the conjugation of the first donor cell and the first recipient cell, the first recipient cell comprises one or more endonuclease specific for C1, C2, and optionally C3, and C4, such that C1, C2, and optionally C3, and C4 are cleaved. After conjugation, cleavage by the endonucleases and following the homologous recombination of HR1 with HR3 and HR2 with HR4, a first recombined polynucleotide in the recipient cell comprising, from 5′ to 3′, a fragment of the first sequence to be truncated, HR1/HR3, optionally, the first joining polynucleotide sequence, the first selection cassette, and HR2/HR4.

In an aspect of the current disclosure, methods of truncating and, optionally, replacing, at least one DNA sequence are provided. In some embodiments, the methods comprise: (a) conjugating a first donor cell with a first recipient cell, wherein the first donor cell comprises a first donor construct and wherein the first recipient cell comprises a first recipient polynucleotide, to (i) transfer the first donor construct from the first donor cell to the first recipient cell and (ii) recombine the first donor construct and the first recipient construct in the first recipient cell by homologous recombination. The first donor construct comprises, from 5′ to 3′, (1) a first endonuclease site (C11), a first sequence to be truncated comprising a first homologous recombination region (HR1), (2) a first selection cassette comprising at least one first selectable marker, (3) a second homologous recombination region (HR2), and (4) a second endonuclease site (C2), wherein HR1 is located at least one bp downstream of the 5′ end of the first sequence to be truncated; the recipient construct comprises, from 5′ to 3′, optionally, a first joining sequence, (1) a third homologous recombination region (HR3) that is homologous to HR1, and (2) a fourth homologous recombination region (HR4) that is homologous to R2, and optionally further comprising a removable selectable construct comprising from 5′ to 3′ an optional third endonuclease site (C3), a second selection cassette comprising at least one second selectable marker, wherein the at least one second selectable marker is distinct from the at least one first selectable marker in the first selection cassette, a fourth endonuclease site (C4); wherein, following the conjugation of the first donor cell and the first recipient cell, the first recipient cell comprises one or more endonuclease specific for C1, C2, and optionally C3, and C4, such that C1, C2, and optionally C3, and C4 are cleaved; thereby providing, following the homologous recombination of HR1 with HR3 and R2 with HR4, a first recombined polynucleotide in the recipient cell comprising, from 5′ to 3′, optionally, the first joining sequence, HR1/HR3, a 3′ fragment of the first sequence to be truncated, the first selection cassette, and HR2/HR4.

In another aspect of the current disclosure, methods of joining two polynucleotide sequences are provided. The method comprises: (a) conjugating a first donor cell comprising a first bridging construct with a first recipient cell comprising a first recipient polynucleotide to (i) transfer the first bridging construct from the first donor cell to the first recipient cell and (ii) recombine the first bridging construct and the first recipient construct in the first recipient cell by homologous recombination. The first bridging construct comprises, from 5′ to 3′, (1) a first endonuclease site (C1), (2) a first homologous recombination region (HR1), (3) a fifth homologous recombination region (HR5), (4) an optional fifth endonuclease site (C5), (5) a first selection cassette comprising at least one first selectable marker, (6) an optional sixth endonuclease site (C6), (7) a second homologous recombination region (HR2), and (8) a second endonuclease site (C2). The first bridging construct may also optionally contain a first sequence to be joined in the bridging construct between HR1 and HR5. The recipient construct comprises, from 5′ to 3′, an optional first joining polynucleotide comprising a third homologous recombination region (HR3); a fourth homologous recombination region (HR4); and optionally further comprising between HR3 and HR4 a removable selectable construct comprising an optional third endonuclease site (C3), a second selection cassette comprising at least one second selectable marker, wherein the at least one second selectable marker is distinct from the at least one first selectable marker in the first selection cassette, and an optional fourth endonuclease site (C4). Following the conjugation of the first donor cell and the first recipient cell, the first recipient cell comprises one or more endonuclease specific for C1, C2, and optionally C3 and C4, such that C1, C2, and optionally C3 and C4 are cleaved. Thereby providing, following conjugation, cleavage with the endonuclease and the homologous recombination of HR1 with HR3 and HR2 with HR4, a first recombined polynucleotide in the recipient cell is generated. The first recombined polynucleotide comprises from 5′ to 3′, the optional portion of the first joining polynucleotide upstream of HR3, HR1/HR3, optionally the first sequence to be joined, HR5, optionally C5, the first selection cassette, optionally C6, and R2/HR4. A second conjugation can then be completed in step (b) which comprises: conjugating a second donor cell comprising a second donor construct to the cell comprising the first recombined polynucleotide to (i) transfer the second donor construct from the second donor cell to the cell comprising the first recombined polynucleotide and (ii) recombine the second donor construct and the first recombined polynucleotide in the recipient cell by homologous recombination. The second donor construct comprises, from 5′ to 3′, (1) a seventh endonuclease site (C7), (2) a second joining polynucleotide sequence comprising a sixth homologous recombination region (HR6) that is homologous to HR5, (3) an optional ninth endonuclease site (C9), (4) a third selection cassette comprising at least one selectable marker, wherein the at least one selectable marker is distinct from the at least one first selectable marker in the first selection cassette, (5) an optional tenth endonuclease site (C10), (6) a seventh homologous recombination region (HR7), and (7) an eighth endonuclease site (C8). The cell comprising the first recombined polynucleotide comprises at least one endonuclease specific for C7 and C8, such that are C7 and C8 are cleaved. Thereby providing, following conjugation, cleavage by the endonuclease for C7 and C8 and the homologous recombination of HR6 and HR5 and HR7 and R2, a second recombined polynucleotide comprising, from 5′ to 3′: the optional portion of the first joining polynucleotide upstream of HR3, HR3, the optional first sequence to be joined, HR5/HR6, the second joining polynucleotide, optionally C9, the third selection cassette, optionally C10, and HR2/HR7.

### BRIEF DESCRIPTION OF THE DRAWINGS

FIGS. 1A, 1B, 1C, 1D, 1E, 1F, 1G, and 1H. Schematic of several embodiments of the disclosed methods for DNA truncation/assembly described herein. Boxes designated C1, C2, etc. refer to endonuclease sites; boxes designated HR1, HR2, etc. refer to homologous recombination regions (which can be internal or terminal to DNA elements); boxes designated with a number and a plus or minus sign refer to selectable (+) and counter-selectable (−) markers. Not all elements shown in the schematic are required in every embodiment of the methods described here with optional elements shown located slightly below neighboring elements, e.g., JPS1, HR7, and C5 in FIG. 1A. FIG. 1A shows an embodiment of the disclosed methods where a 3′ end of the first sequence to be truncated (1stSTBT) is removed. “JPS1” refers to an optional first sequence to be joined (first joining polynucleotide sequence). (B) FIG. 1B shows an embodiment of FIG. 1A, which further includes an optional removable selection cassette flanked by C5 and C6. (C) FIG. 1C shows an embodiment of the method in FIG. 1A further comprising an additional round of “stitching” with an optional second joining polynucleotide sequence added to the 3′ end of the JPS1. (D) FIG. 1D shows an embodiment of the method shown in FIG. 1A, further requiring the optional JPS1, thus performing both a 3′ truncation of 1stSTBT and joining of the JPS1. (E) FIG. 1E shows an embodiment of the method employed to truncate the 5′ end off of an incoming DNA element located in a donor construct. In this embodiment, the 1stSTBT is located in the donor construct and, following recombination, the 5′ end of the 1stSTBT is removed. This embodiment also provides an optional first joining polynucleotide sequence (1stJS), located in the donor vector, upstream of HR3. (F) FIG. 1F shows an embodiment of the method shown in FIG. 1E further comprising the optional C5 and C6 endonuclease sites flanking the selection cassette. (H) FIG. 1G shows an embodiment where the donor cells comprises a “bridging construct.” The bridging construct comprises HR1 (which is homologous to HR3 in the recipient construct) and HR5 (which is homologous to another construct or element) such that the recombined polynucleotide has a homologous recombination region that is homologous to another polynucleotide element (e.g., JPS2), even when no homology exists in the two initial constructs. Further, the HRs may be designed such that one or both of the joining sequences (JPS1 or JPS2) are truncated.

FIGS. 2A, 2B, and 2C are a schematic overview of example methods for truncation of an oligonucleotide or assembly without PCR. O1: Oligonucleotide 1; T1: Truncation oligonucleotide 1, which recombines with an internal site on an oligonucleotide introduced on a donor plasmid and removes a segment from the front of the oligonucleotide; T2: Truncation oligonucleotide 2, which recombines with an internal site on an oligonucleotide or assembly in a recipient plasmid in a recipient cell and removes a segment from the back of the oligonucleotide or assembly; H1: a universal homology region on a recipient plasmid without an assembly, which is used as a recombination site to introduce the first DNA block in an assembly or truncation method. (A) A donor plasmid containing truncation oligonucleotide T2 is conjugated to a recipient cell containing oligonucleotide O1 in a recipient plasmid such that recombination with T2 truncates a segment from the back of O1 (producing O1*). (B) A segment from the front of the oligonucleotide O1 is removed by first conjugating a DNA block containing a homology region H1 and a truncation oligonucleotide T1 from donor cells to recipient cells such that T1 is introduced into the recipient plasmid. Next, a donor plasmid containing O1 is conjugated to recipient cells such that recombination with T1 truncates a segment from the front of O1 (producing *O1). (C) A segment from the front and back of the oligonucleotide O1 is removed. As in (B), a DNA block containing a homology region H1 and a truncation oligonucleotide T1 from donor cells to recipient cells such that T1 is introduced into the recipient plasmid. Next, a donor plasmid containing O1 is conjugated to recipient cells such that recombination with T1 truncates a segment from the front of O1 (producing *O1). Next, a donor plasmid containing truncation oligonucleotide T2 is conjugated to recipient cells such that recombination with T2 truncates a segment from the back of O1 (producing *O1*).

FIGS. 3A, 3B, and 3C are a schematic overview of example methods for joining and truncating oligonucleotides with respective HR-regions, which are referred to in this figure as bridging oligonucleotides (“BO”). O1: Oligonucleotide 1; O2: Oligonucleotide 2; BO1: Bridging Oligonucleotide 1; BO2: Bridging Oligonucleotide 2. (A) A segment from the back of the O1 is removed and O1 is joined with O2 by first conjugating a DNA block containing BO1 and BO2 from donor cells to recipient cells such that recombination with BO1 truncates a segment from the back of O1 (producing O1* adjacent to BO2). Next, a donor plasmid containing O2 is conjugated to recipient cells. Recombination between BO2 and the terminal region of O2 produces an assembly containing a truncated O1 adjacent to a full-length O2. (B) A segment from the front of the O2 is removed and O2 is joined with O1 by first conjugating a DNA block containing BO1 and BO2 from donor cells to recipient cells such that recombination between BO1 and a terminal region of O1 results in a full-length O1 adjacent to BO2. Next, a donor plasmid containing O2 is conjugated to recipient cells. Recombination between BO2 and an internal region of O2 truncates a segment from the front of O2 (producing *O2) resulting in an assembly containing a full-length O1 adjacent to a truncated O2. (C) A segment from the back of O1 and the front of the O2 are removed and truncated O1 is joined with truncated O2. First, a DNA block containing BO1 and BO2 is conjugated from donor cells to recipient cells such that recombination between BO1 and a terminal region of O1 results in a truncated O1 (O1*) adjacent to BO2. Next, a donor plasmid containing O2 is conjugated to recipient cells. Recombination between BO2 and an internal region of O2 truncates a segment from the front of O2 (producing *O2) resulting in an assembly containing a truncated O1 adjacent to a truncated O2.

FIG. 4 is a schematic overview of an example of an assembly that uses respective HR-regions, which are referred to in this figure as bridging oligonucleotides (“BO”) to truncate and join oligonucleotides that were designed for different assemblies. O1: the first DNA block of a 13-block Malbranchea aurantiaca malbrancheamide biosynthetic gene cluster assembly; O2: the first DNA block in a 3-block 9 kb assembly of synthetic DNA; O3: the second DNA block in a 2-block 10 kb assembly of synthetic DNA; O4: the fifth DNA block of a 13-block Malbranchea aurantiaca malbrancheamide biosynthetic gene cluster assembly. BO1-BO6: bridging oligonucleotides. (A) A segment from the back of the O1 is removed by recombination with an oligonucleotide that contains BO1-BO2. (B) O2 is added to the assembly by recombination with BO2. (C) A segment from the back of the O2 is removed by recombination with an oligonucleotide that contains BO3-BO4. (D) O3 is added to the assembly by recombination with BO4. Recombination occurs at an internal site on O3, removing a segment from the front of O3. (E) An oligonucleotide that contains BO5-BO6 is added to an assembly. Recombination between BO5 and the terminal region of O3 adds BO6 without a truncation to the end of O3. (F) O4 is added to the assembly by recombination with BO6. Recombination occurs at a terminal site on O4, resulting in assembly without a truncation to O4. (G) Purity scores (fraction of sequence perfect molecules) from 12 replicate assemblies following procedures A through F.

FIG. 5 is a schematic overview of an example workflow for construction of an arrayed library of donor plasmids in donor cells that contain a segment of natural DNA. Genomic DNA is digested, sonicated, or fragmented to produce a linear DNA library. The linear DNA library is integrated into donor plasmids and this plasmid library is transformed into donor cells. Clones resulting from this transformation are isolated and DNA inserts are sequenced. Selected clones are then arrayed to produce a natural DNA repository for DNA assembly.

FIG. 6 is a schematic overview of an example workflow for engineering with existing part libraries and respective HR-regions, which are referred to in this figure as bridging oligonucleotides (“BO”). Genomic DNA part arrays are re-arrayed into new positions to construct the desired assembly. Oligonucleotides each containing a pair of Bridging Oligonucleotides (BO1 and BO2) that are homologous to terminal or internal sequences of genomic DNA parts are synthesized, integrated into donor plasmids, transformed into donor cells, and arrayed to be compatible with the preceding and following genomic DNA arrays. DNA assembly proceeds with alternating genomic DNA parts and bridging oligonucleotides. Bridging oligonucleotides may truncate a segment from the front (e.g. *GP2), the back (e.g. GP1*), or the front and back (e.g. *GP2*, not shown) of a genomic DNA part, resulting in only the desired part of an existing DNA sequence being incorporated into an assembly.

FIG. 7 is a schematic overview of a branching DNA assembly using composable DNA elements, where bridging blocks (B10, B11) are designed with homology to the middle of a DNA assembly and/or incoming DNA block to join selected internal parts of existing blocks.

FIGS. 8A, 8B, and 8C. SCRIVENER schematic and fidelity. A. Arrays of input DNA blocks are introduced into donor plasmids and donor cells using standardized methods to create donor plates. In each assembly round, a DNA cassette is transferred from a donor plasmid to a recipient plasmid to extend a growing assembly. Two types of donor plasmids, used on alternating rounds, are transferred from donor cells to recipient cells via F-plasmid (not shown) mediated conjugation. Donor and recipient plasmids are cut in the recipient cell using CRISPR/Cas9 (scissors), guided by alternating gRNAs on the donor plasmids (not shown). Homology regions (HRs) promote recombination and seamlessly stitch input DNA together. Alternating selectable (+1, +2) and counter-selectable (−1, −2) markers on donor plasmids allow selection for sequential DNA transfers. B. Barplot showing the number of fluorescent (dark colors) and non-fluorescent (light colors) colonies following selection (blue) and counter-selection (red) for the last step in a liquid mPapaya assembly when the length of homology between the blocks is varied. Error bars show the standard deviation. C. Results from replicate four-part assemblies of mPapaya (fluorescent yellow colonies) and lacZ (blue colonies) grown on X-gal and visualized under blue light.

FIGS. 9A and 9B. SCRIVENER performance with long and challenging DNA sequences. A. DNA blocks of different lengths (gray boxes) were assembled by sequential conjugation and selection on 96- or 384-position arrays at 12-fold replication. Plasmids in the resulting colonies were verified using high coverage long-read Oxford Nanopore sequencing. The purity score (heatmap) for each replicate indicates the fraction of sequence-perfect DNA molecules. B. Sequence feature (top) and read depth (bottom) plots of some replicates of long and/or complex assembled constructs. Regions that BLAST against each other (triangles) indicate long interspersed repeats, while those with high hairpin scores indicate DNA secondary structure. GC content is the GC % in a 51 bp rolling window. Regions with extreme GC % (<30% or >70%) and extreme secondary structure (hairpin score<−2,500) are highlighted in the lower half of each strip (gray box). Deletions between long interspersed repeats can be observed by a drop in read coverage in a replicate assembly.

FIGS. 10A and 10B. SCRIVENER deletion dynamics. A. Heatmaps of replicate assembly purity scores at different stages of assembly for biosynthetic gene clusters. Each row is a different assembly replicate, and each column is a different stage of assembly (stitch number). B. Sequence feature (top) and read depth (bottom) plots of a replicate of the Aspergillus flavus cyclopiazonic acid biosynthetic gene cluster (highlighted in red in panel A) at different stages of assembly.

FIGS. 11A, 11B, and 11C. Assembly of combinatorial libraries in arrays and pools. A. Arrayed assembly of 1,296 combinatorial GPCR designs. Designs are ranked by overall length. Colors indicate the length of each block, with homologous overlaps shown in gray. Heatmap is the purity score for the four replicate assemblies of each design. B. Ribbon diagram of an assembled GPCR variant. Colors correspond to the blocks shown in panel A, with homologous overlaps shown in gray. c. Muller plot showing the relative frequency of each design over the course of a pooled assembly.

FIGS. 12A, 12B, 12C, and 12D. Composable DNA with bridging blocks. A. DNA blocks from different assemblies (numbered gray boxes) that lack homology to each other are seamlessly joined with 80-100 bp bridging blocks (B1-B9, blue boxes). The third assembly with low purity scores contains a 2,519 bp imperfect repeat between the synthetic DNA blocks, which is associated with frequent deletions. B. Bridging blocks (B10, B11) are designed with homology to the middle of an assembly or incoming block to join selected parts of existing blocks. C. Two complete assemblies are joined with a bridging block (B13). D. Ten assemblies are joined with nine bridging blocks (B13-B21). Sequencing is performed before single-cell isolation for A-C and after single-cell isolation for D. Heatmaps show the purity score for each replicate assembly.

FIG. 13. The histogram of read lengths for a replicate assembly of the Scytonema scytodecamide stitch number 5 shows two distinct “peaks”. These peaks presumably represent sequence data from fully intact plasmid DNA molecules that were not fragmented during plasmid extraction and library preparation, and suggests that there are at least two distinct plasmids within this replicate assembly. The red line is the read number threshold used to identify a peak. Any reads within 150 bp of these peaks were assigned to the closest peak to form “clusters”. In this example, two clusters with sizes of 12,373 bp (132 reads) and 14,173 bp (68 reads) were identified. The expected sequence length of this construct with the plasmid backbone is 14,174 bp, suggesting that one of the plasmids has a ˜1,800 bp deletion while the other plasmid is the expected size. To identify any sequence variants in these plasmids, reads in each cluster were analyzed individually using bcftools and Sniffles2 (Methods). Using these data, purity score, defined as the fraction of plasmid molecules that are both full-length and have perfectly assembled sequences, was determined by calculating the fraction of reads assigned to the expected size cluster relative to the total number of reads assigned to all clusters. If any sequence variants were detected in the assembled product of the plasmid with the expected size, the purity score was set to 0. In this example, no sequence variants were detected in the plasmid with the expected size, and therefore, the purity score is 34% (68/(132+68)).

FIG. 14. Density plots comparing the maximum blast score of 117 deletion junctions found in the long and difficult DNA test set (red) with random junctions (blue). The random junctions are separated by the same distance as the deletions and were chosen from the same construct where the deletion was detected.

FIGS. 15A, 15B, and 15C. Muller plots showing the frequency distribution of each assembly product at every stitch for pool assembly replicates 1-1 (a), 2-1 (b), and 2-2 (c). Replicate 1-2 is shown in FIG. 11C.

FIGS. 16A, 16B, 16C, and 16D. The frequency of each DNA block in the final pool for each pooled assembly replicate. Colors represent the relative frequency of each block. The expected value is 0.167 (1/6) for each block.

FIGS. 17A, 17B, 17C, and 17D. Scatter plot of the relative read frequency of each pooled assembly replicate across four stitches: (a) Stitch 1, (b), Stitch 2, (c) Stitch 3, and (d) Stitch 4. Technical replicates are 1-1 vs. 1-2 and 2-1 vs. 2-2, and biological replicates are 1-1 vs. 2-1, 1-1 vs. 2-2, 1-2 vs. 2-1, and 1-2 vs. 2-2. Blue line and red text indicate the Pearson correlation (R).

FIG. 18. A schematic showing several different applications of the truncating, joining and bridging methods provided herein.

### DETAILED DESCRIPTION

Recombineering DNA Blocks and DNA Assemblies without PCR

The inventors discovered methods to truncate existing DNA sequence and/or incorporate new sequences in a single method step. The disclosed methods are provided as several distinct embodiments.

### Truncating/Removing a 3′ Segment of a Polynucleotide

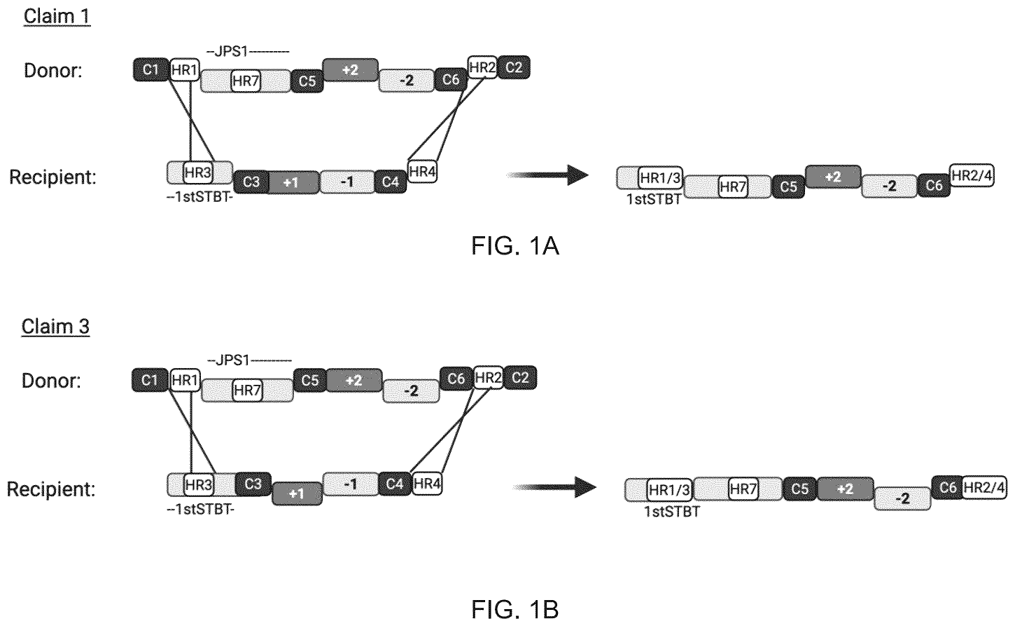

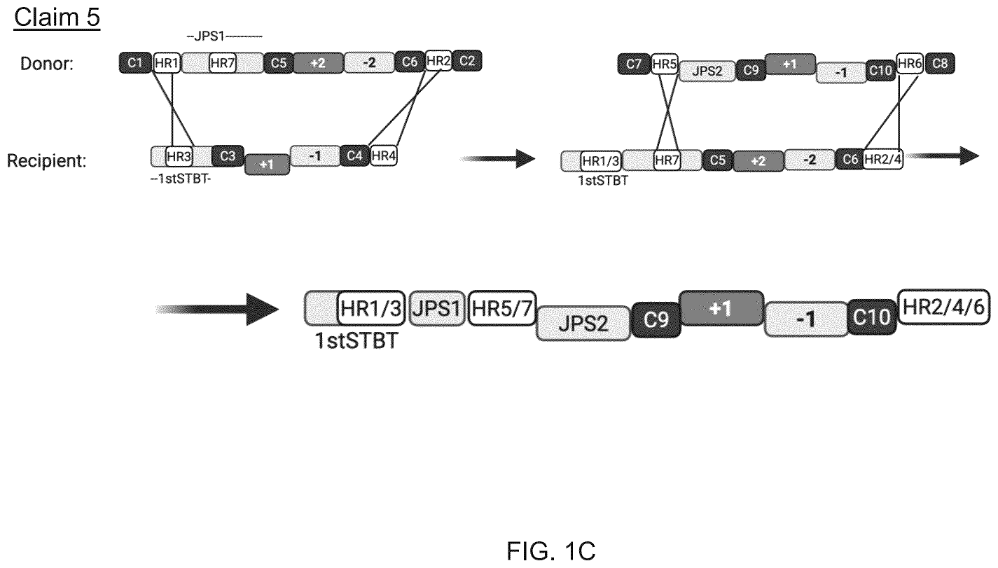

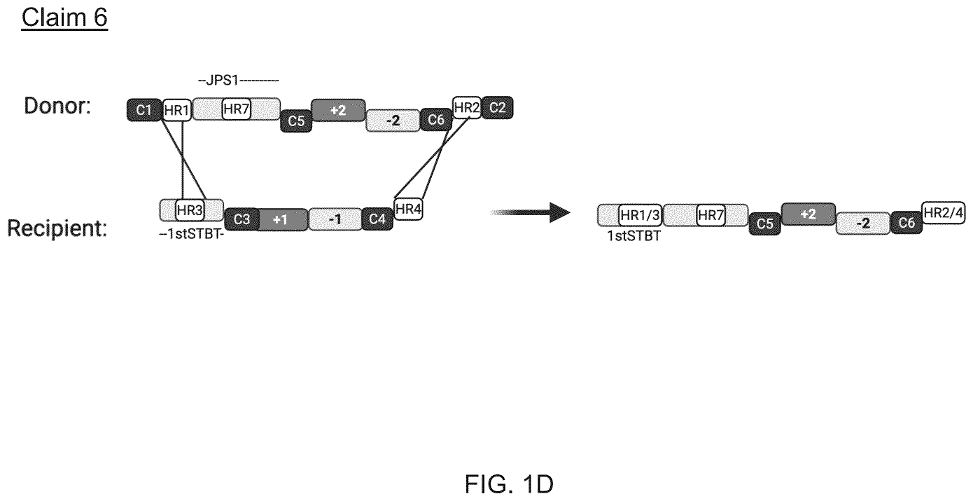

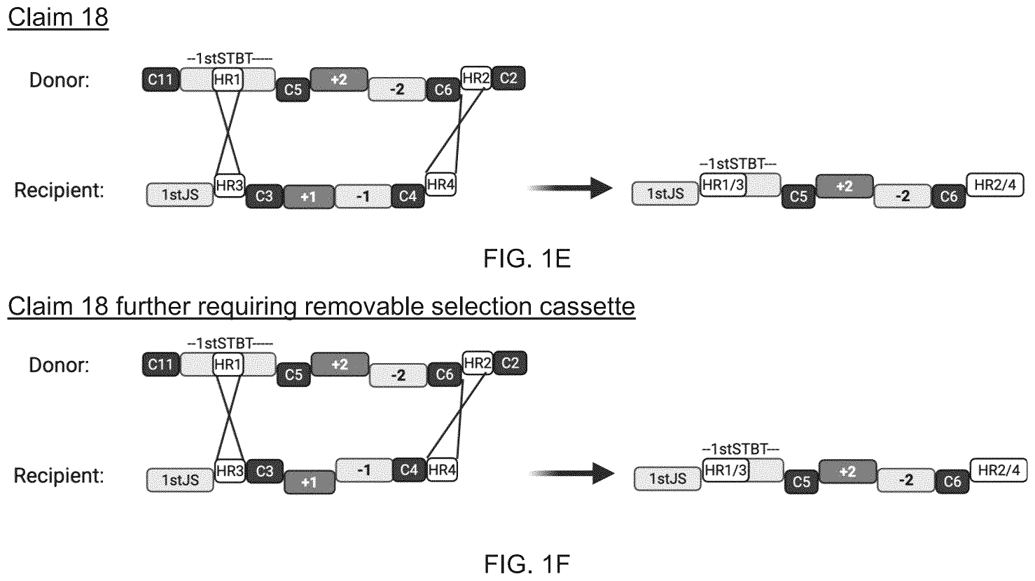

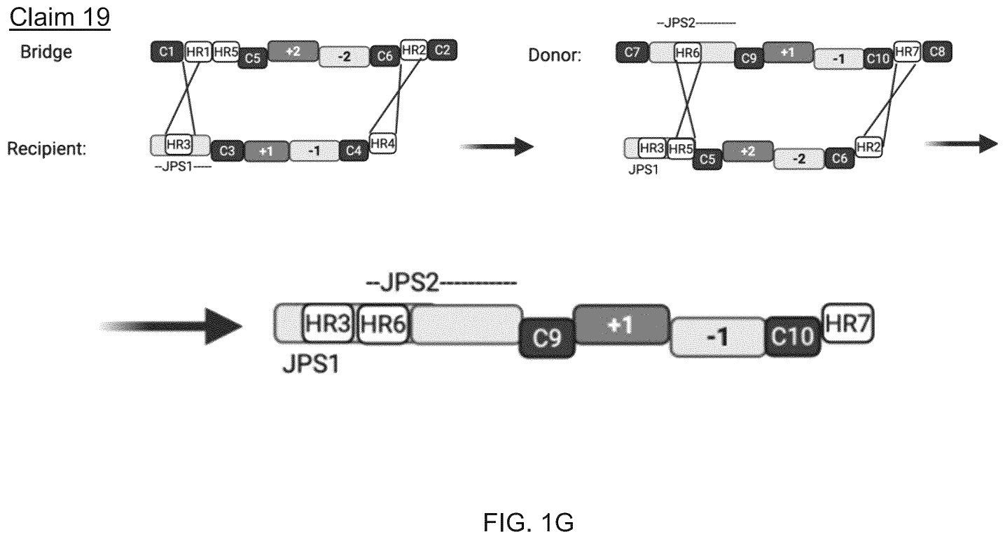

Referring now to FIG. 1, the methods comprise mating two bacterial cells, a donor cell and a recipient cell, where the donor cell comprises a donor construct and the recipient cell comprises a recipient construct. The donor construct is designed to comprise, from 5′ to 3′, a first endonuclease site, a first homologous recombination region (HR1), an optional first joining sequence, an optional fifth endonuclease site, a selection cassette, an optional sixth endonuclease site, a second homologous recombination region (HR2 in FIG. 1), and a second endonuclease site.

The recipient construct comprises a homologous recombination region (HR3) located at least one bp from the 5′ end of a sequence to be truncated (referred to as first sequence to be truncated (1stSTBT) in FIG. 1). And a fourth homologous recombination region (HR4) located 3′ of the sequence to be truncated.

Through conjugation, the donor plasmid is transferred to the recipient cell and the endonuclease sites (C1, C2, and optionally C3 and C4) are cut to (i) optionally open the recipient construct and (ii) free the donor sequence from the donor construct by cleaving at the endonuclease sites C1 and C2. Leveraging homologous recombination machinery present in the recipient cell, the homologous regions (HR1/HR3 and HR2/HR4) recombine to truncate off a portion of the sequence to be truncated that is downstream of the 3′ end of HR3 and introduce a new selectable cassette into the recipient construct, allowing for selection of the recombined vector comprising the truncated sequence. A portion of a sequence includes any portion greater than 1 bp on either side of the HR region and maintained after the conjugation, cleavage and recombination events of the method. The HR regions may be designed to allow for portions of sequences to be maintained after the method is carried out in the recipient recombined polynucleotide or to be transferred from the donor to the recombined polynucleotide. Those of skill in the art will recognize that based on the design of the HR regions the sequence of the recombined polynucleotide can be controlled.

Accordingly, provided herein are methods of truncating and, optionally, replacing at least one DNA sequence. In some embodiments, the methods comprise (a) conjugating a first donor cell comprising a first donor construct with a first recipient cell comprising a first recipient polynucleotide to (i) transfer the first donor construct from the first donor cell to the first recipient cell and (ii) recombine the first donor construct and the first recipient construct in the first recipient cell by homologous recombination. The first donor construct comprises, from 5′ to 3′, (1) a first endonuclease site (C1), (2) a first homologous recombination region (HR1), optionally, a first joining polynucleotide sequence, which optionally comprises a seventh homologous recombination region (HR7), (3) a first selection cassette comprising at least one first selectable marker, (4) a second homologous recombination region (HR2), and (5) a second endonuclease site (C2). The recipient construct comprises, from 5′ to 3′, (1) a first recipient polynucleotide comprising a first sequence to be truncated comprising a third homologous recombination region (HR3) that is homologous to HR1, and (2) a fourth homologous recombination region (HR4) that is homologous to HR2, and, optionally, further comprising a removable selectable construct comprising from 5′ to 3′ a third endonuclease site (C3), a second selection cassette comprising at least one second selectable marker, wherein the at least one second selectable marker is distinct from the at least one first selectable marker in the first selection cassette, and a fourth endonuclease site (C4), wherein HR3 ends at least one base pair (bp) upstream of the 3′ end of the first sequence to be truncated. Following the conjugation of the first donor cell and the first recipient cell, the first recipient cell comprises one or more endonuclease specific for C1, C2, and optionally C3, and C4, such that C1, C2, and optionally C3, and C4 are cleaved by the endonuclease. Thereby providing, following the conjugation, cleavage and homologous recombination of HR1 with HR3 and HR2 with HR4, a first recombined polynucleotide in the recipient cell. The first recombined polynucleotide comprises, from 5′ to 3′, a fragment of the first sequence to be truncated, HR1/HR3, optionally, the first joining polynucleotide sequence, the first selection cassette, and HR2/HR4.

The recipient cells comprising the first recombined sequence may be selected for by selecting for the selectable marker in the first selection cassette recombined from the first donor construct.

The “homologous recombination regions” or “HRs” as used herein, refer to regions that have the proper orientation and sequence homology to allow for homologous recombination to occur. For example, homologous HRs may have, in some embodiments, at least 90%, 91%, 92%, 93%, 94%, 95%, 96%, 97%, 98%, 99%, or 100% homology to allow recombination between two sets of sequences flanked by homologous regions to recombine. Thus, for an exemplary HR that is 20 bp in length, HRs have at least 18 nucleotides in common.

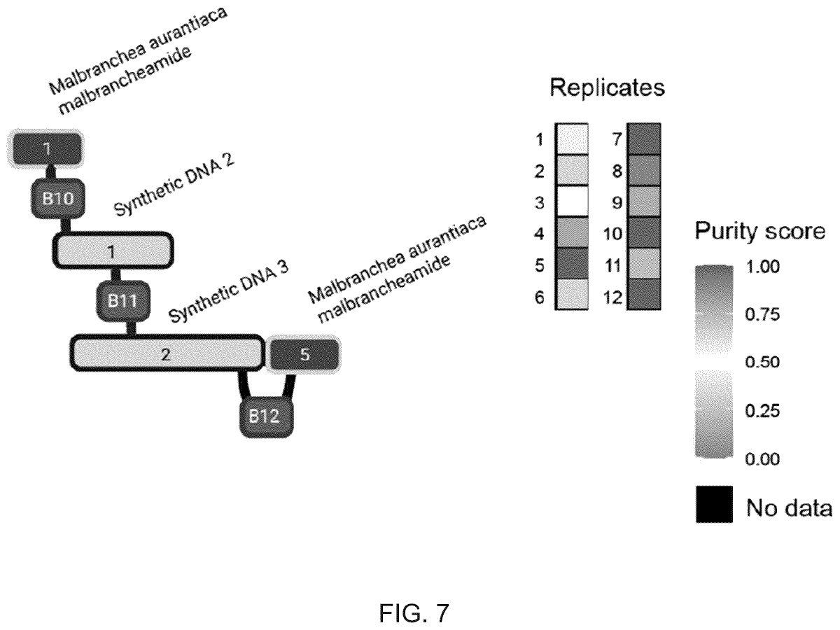

The HRs may be, e.g., greater than about 10 bp to about 40 bp, 50 bp, 60 bp, 80 bp, 100 bp, or more. The inventors demonstrated that HRs of about 40 bp are sufficient for about 100% recombination. See, e.g., FIG. 7D of WO2022187697A1. The homologous regions should be designed to not be highly homologous to other regions in the recipient or donor constructs to increase the chance of successful recombination. The first, second or subsequent homologous recombination (HR) regions and their corresponding HR regions on the recipient polynucleotide may each have a length in a range selected from the group consisting of: from 20-1000, from 30-900, from 40-800, from 50-700, from 50-600, from 50-500, from 50-400, from 50-300, from 50-200, from 60-150, from 70-125, and/or from 80-100 nucleotides in length. The length of the homology region may depend at least in part on the length of the DNA segment being added by the donor construct i.e., the length of DNA between HR1 and HR2 in the donor construct with longer DNAs requiring longer HRs.

In certain embodiments, the HRs are located within sequences to be truncated. Depending on the particular method, the region either 5′ to or 3′ to the HR is truncated. For example, in FIG. 1A, HR3 is located within the first sequence to be truncated (1stSTBT), and, because the 1stSTBT is located in the recipient vector, the 3′ end following HR3 is truncated off during homologous recombination. In contrast, referring to FIG. 1E, HR1 may be located within the incoming donor construct (1stSTBT) and, therefore, a region 5′ to HR1 in the 1stSTBT is removed by homologous recombination generating a 3′ fragment of the 1stSTBT.

The endonuclease sites, as used herein, may be any polynucleotide sequence that is specifically recognized by an endonuclease. For example, the endonuclease sites may be recognized by a RNA-guided DNA nuclease, e.g., a Cas nuclease, or a Cas-like nuclease, in complex with a guide RNA (gRNA). RNA-guided DNA nucleases (also referred to herein as endonucleases) are discussed further below. Thus, the endonuclease site may comprise or consist of a gRNA targeting sequence, e.g., a 20-mer, in the case of Streptococcus pyogenes Cas9 (spCas9). These endonuclease sites need to be designed so that they are unique sequences as well to avoid targeting DNA non-specifically.

The expression of the endonuclease and/or gRNA may be inducible. Thus, the skilled person may design constructs such that endonuclease digestion only occurs following conjugation and induction as a two-step control on the endonuclease to prevent unwanted cutting. For example, a donor construct may comprise sequences encoding gRNAs and the recipient cell may comprise a construct comprising a sequence encoding the RNA-guided DNA nuclease operably linked to an inducible promoter. Following conjugation, the recipient cell may produce gRNAs but, in the absence of induction of the RNA-guided DNA nuclease, no cutting occurs. Therefore, inducing the expression of the nuclease leads to sequence-specific cutting and recombination in the recipient cell. The gRNAs may each bind to one endonuclease site, or gRNAs may target multiple endonuclease sites in the constructs.

A “construct,” as used herein, refers to a DNA polynucleotide sequence. Constructs include, but are not limited to, plasmids, minicircles, bacterial artificial chromosomes (BACs), yeast artificial chromosomes (YACs), etc.

The endonuclease sites may also be sites for endonuclease digestion by, e.g., TAL effector nucleases (TALENs), traditional restriction enzymes, or any other suitable specific endonuclease. Further, for each “round” of recombination, the endonuclease sites may be identical and may use the same endonuclease and/or gRNA. The endonucleases are within the recipient cell for each round of recombination. There are at least three exemplary strategies: the endonuclease (or a polynucleotide encoding the same) is transferred from the donor cell to the recipient cell during conjugation, the recipient cell comprises a polynucleotide encoding the endonuclease on a self-replicating non-chromosomal polynucleotide, e.g., a plasmid, or the endonucleases are chromosomally encoded. The same strategies may be used to deliver/provide gRNAs, where applicable, in the recipient cell for one or more rounds of recombination. The key feature is that the endonuclease and the targeting sequences (such as gRNAs) should only be expressed together in the recipient cells after transfer of the donor construct to the recipient cell.

An exemplary use for the disclosed methods is the truncation and/or joining of DNA elements from, e.g., genomic DNA or a cDNA library. Therefore, a sequence to be truncated may comprise an open reading frame (ORF) and may, e.g., be flanked by 5′ and 3′ UTRs. The disclosed methods provide a scalable and efficient scheme for transforming libraries into usable blocks by, in one example, removing UTRs to isolate only an ORF. In some embodiments, HR3 is located downstream of a 3′ untranslated region (3′ UTR) in the at least one ORF or wherein the HR4 is located upstream of a 5′ untranslated region (5′ UTR) in the at least one ORF.

The disclosed methods utilize homologous recombination to truncate and/or join DNA elements. Therefore, in some embodiments, the recipient cell, which is the location for the recombination events, comprises the machinery necessary to effect recombination, also referred to herein as “one or more homologous DNA repair genes.” For example, the recombination machinery may comprise the lambda red system. Thus, the recipient cell may comprise Gam, Exo, and Beta, of the lambda red system. As with the endonuclease or gRNAs, a polynucleotide comprising sequences encoding the lambda red machinery may be delivered to the recipient cell by conjugation, encoded on a self-replicating non-chromosomal polynucleotide, e.g., a plasmid, or be chromosomally encoded.

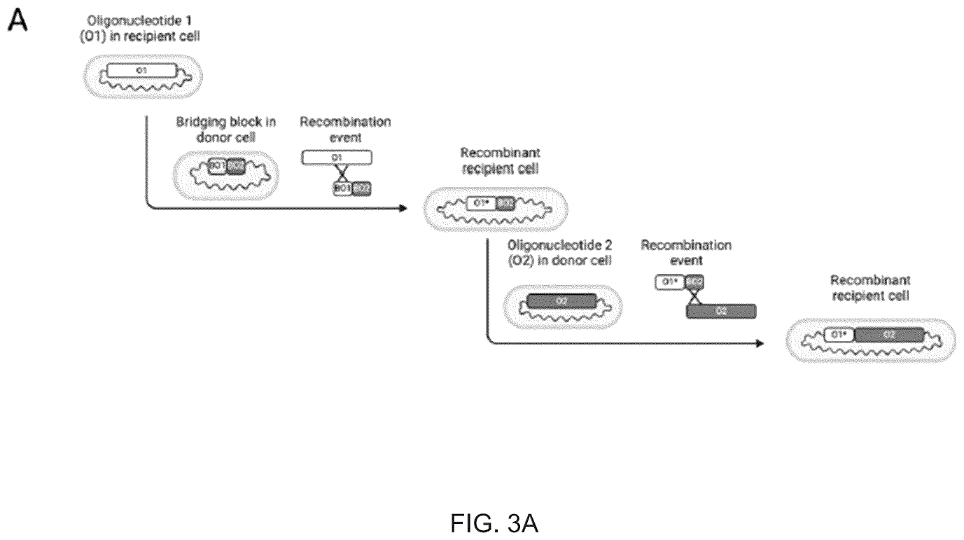

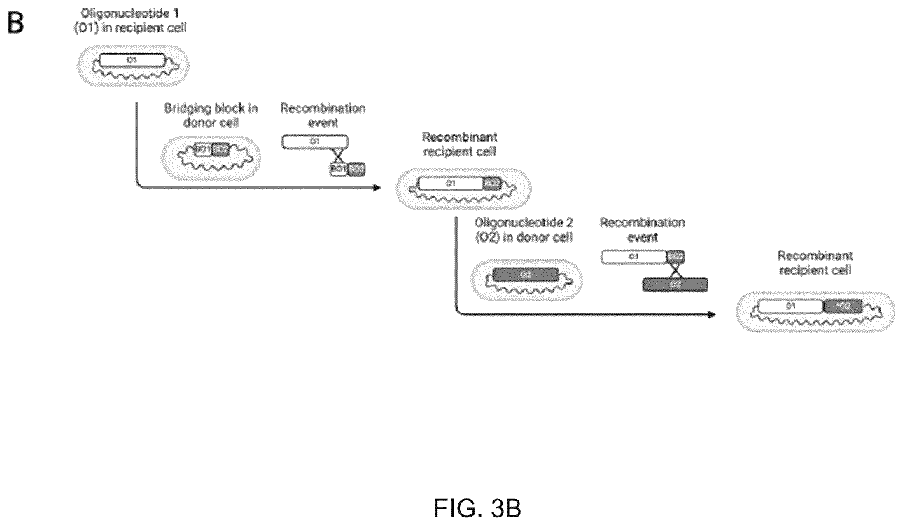

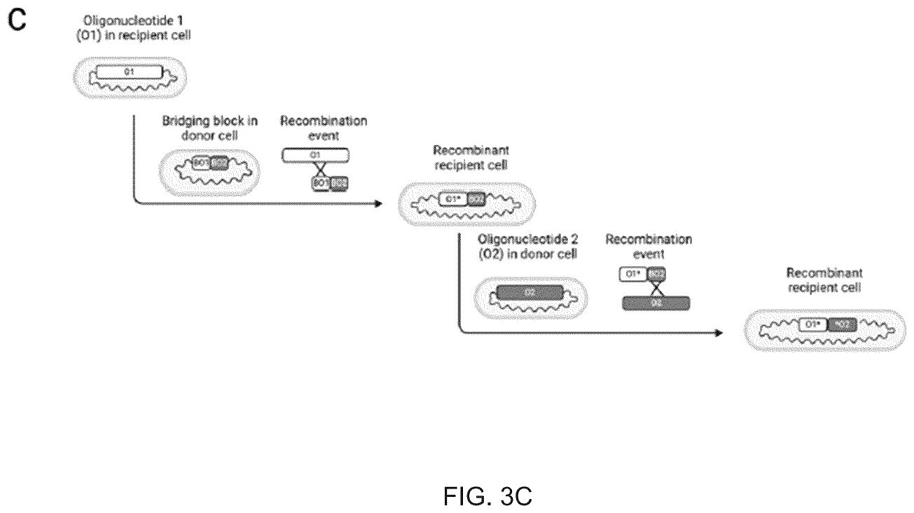

Recombination systems utilized by the present invention, such as the lambda red recombination and endogenous E. coli recombination systems, have been shown to function distant from a double stranded break. For example, E. coli recombineering systems use lambda red recombination to insert small DNA sequences at nearly any location on a 4 Mb circular bacterial genome. Because of these features, provided herein for use in the invention methods are bridging blocks (e.g., set forth in FIGS. 1-3) that can be internally recombined at sites on recipient oligonucleotides (“recombined-recipient oligonucleotide) that are distant from the terminal ends that are produced with CRISPR/Cas9, such as for example, 1 kb, 10 kb, 100 kb, 500 kb, or the like, away from a terminal end of existing DNA elements, DNA assemblies, partial DNA assemblies under stitching with the invention methods, incoming DNA blocks being used in the invention methods. This capability permits the creation and use of repositories composed of arrays of long DNA inserts (e.g., 1-500 kb are contemplated) stored on donor plasmids in donor cells (e.g., YAC or BAC libraries). In accordance with the present invention, any DNA element of a DNA insert from any plasmid in a repository can be assembled into a growing assembly on a recipient plasmid with one or more bridging blocks (e.g., HR-region) that provide homology to the respective regions of a desired DNA fragment. Several different types of repositories are useful for high throughput DNA engineering using the invention methods, including: 1) libraries of donor plasmids that together constitute the full genomic sequence of an organism (e.g. a BAC library of the human genome); 2) libraries of environmental DNA with utility for bioengineering (e.g. DNA from all organisms found in a hot spring or other extreme conditions); 3) libraries of natural or synthetic long DNA constructs (e.g. cDNAs that contain a 5′ UTR, open reading frame, and 3′ UTR) that can be broken down further into useful fragments (e.g. incorporating only the open reading frames from a cDNA repository). It is contemplated that those of skill in the art will have multiple such libraries available either locally, hosted in centralized repositories, or through a marketplace where collections or individual DNA constructs are commercially available.

In some embodiments, the recipient cell or the donor construct comprises one or more homologous DNA repair genes, e.g., the lambda red homologous repair genes, e.g., (Gam, Exo, and Beta).

The disclosed constructs, e.g., plasmids or other suitable polynucleotide, may contain at least one selectable marker. The disclosed methods may use “selection cassettes” comprising at least one selectable marker. The selection cassettes may comprise sequences encoding 2, 3, 4, 5, 6, or more selectable markers. For example, the selection cassettes may comprise sequences encoding one positive selectable marker, e.g., an antibiotic resistance factor, and one negative or counter selectable marker, e.g., SacB which encodes levansucrase from B. subtilis. The use of selection markers is routine in the art of molecular biology and microbiology.

Selectable markers for use in the methods described herein may be any suitable selectable marker, including auxotrophic markers, and the like. In embodiments, and without limitation, the selectable marker is AmpR, StrR, SpcR, HygR, NsrR, ZeoR, TetA, CmR, SpR, GmR, mFabI, TmR, neoR, Leu, His, Trp, or kanR. In embodiments, the selectable marker is HygR. In embodiments, the selectable marker is NsrR. In embodiments, the selectable marker is ZeoR. In embodiments, the selectable marker is TetA. In embodiments, the selectable marker is CmR. In embodiments, the selectable marker is SpR. In embodiments, the selectable marker is GmR. In embodiments, the selectable marker is mFabI. In embodiments, the selectable marker is TmR. In embodiments, the selectable marker is neoR. In embodiments, the selectable marker is kanR. The positive-selectable marker may comprise an antibiotic resistance factor or an auxotrophic factor.

The donor construct may comprise a conditional origin of replication, wherein replication of the donor construct is dependent on a conditional replication factor, and wherein the first recipient cell does not comprise the conditional replication factor.

The recombined polynucleotide may be in a construct selected from the group consisting of: a yeast artificial chromosome (YAC), a bacterial artificial chromosome (BAC), a mammalian artificial chromosome (MAC), a human artificial chromosome (HAC), and/or a plant artificial chromosome. In some embodiments, the donor construct comprises a replicon that can replicate constructs of lengths greater than 30 kilobases. In some embodiments, the recombined polynucleotide is from about 100 nucleotides to about 500,000 nucleotides in length

The donor construct may comprise an inducible high-copy replication origin.

The donor cell and the recipient cell may be independently a bacteria cell, e.g., E. coli, Vibrio natriegens, or V cholerae.

In accordance with the methods described here, an oligonucleotide, plasmid or vector may contain at least one counter-selectable marker, e.g., one selecting for integration of the second or subsequent oligonucleotide into a recombined recipient oligonucleotide in the methods of assembling a DNA element described here. Counter-selectable markers for use in the methods described herein may be any suitable counter-selectable marker. In embodiments, and without limitation, the counter-selectable marker is PheS, SacB rpsL, pyrF, MazF, lamB, dnaC(Tc), tolC, galK, ccdB, tetA, thyA, lacY, gata-1, URA3, relE, mqsR, chpB, vhaV, or tse2. In embodiments, the counter-selectable marker is PheS. In embodiments, the counter-selectable marker is SacB. In embodiments, the counter-selectable marker is rpsL. In embodiments, the counter-selectable marker is tolC. In embodiments, the counter-selectable marker is galK. In embodiments, the counter-selectable marker is ccdB. In embodiments, the counter-selectable marker is ccdB. In embodiments, the counter-selectable marker is tetA. In embodiments, the counter-selectable marker is thyA. In embodiments, the counter-selectable marker is lacY. In embodiments, the counter-selectable marker is gata-1. In embodiments, the counter-selectable marker is URA3. In embodiments, the counter-selectable marker is relE. In embodiments, the counter-selectable marker is mqsR. In embodiments, the counter-selectable marker is chpB. In embodiments, the counter-selectable marker is vhaV. In embodiments, the counter-selectable marker is tse2.

The donor constructs may comprise an origin of transfer (oriT). Suitable oriTs are known in the art and may be selected by a skilled artisan.

In some embodiments, the methods further comprise one or more steps of lysing the recipient cell; amplifying an assembled DNA element; isolating an assembled DNA element; isolating a recipient polynucleotide; sequencing an assembled DNA element; and sequencing a recipient recombined polynucleotide. In some embodiments, the methods further comprise utilizing two or more recipient polynucleotides having compatible homologous recombination regions to construct a DNA library.

### Successive Rounds of Recombination

The disclosed methods may each be performed reiteratively, allowing successive rounds of stitching to generate fusion constructs. See, e.g., FIGS. 1C and 1G.

Accordingly, the methods may further comprise (b) conjugating a second donor cell comprising a second donor construct to the cell comprising the first recombined polynucleotide to (i) transfer the second donor construct from the second donor cell to the cell comprising the first recombined polynucleotide and (ii) recombine the second donor construct and the first recombined polynucleotide in the recipient cell by homologous recombination, wherein: the second donor construct comprises, from 5′ to 3′, (1) a seventh endonuclease site (C7), (2) a fifth homologous recombination region (HR5) that is homologous to HR7, optionally, a second joining polynucleotide sequence, (3) an optional ninth endonuclease site (C9), (4) a third selection cassette comprising at least one selectable marker, wherein the at least one selectable marker in the third selection cassette is distinct from the at least one first selectable marker in the first selection cassette, (5) an optional tenth endonuclease site (C10), (6) a sixth homologous recombination region (HR6) that is homologous to HR2/HR4, and (7) an eighth endonuclease site (C8); wherein the cell comprising the first recombined polynucleotide comprises at least one endonuclease specific for C7 and C8, such that are C7 and C8 are cleaved; thereby providing, following the homologous recombination of HR5 and HR7 and also HR6 and HR2/HR4, a second recombined polynucleotide, the second recombined polynucleotide comprising, from 5′ to 3′, a fragment of the first sequence to be truncated, HR1/HR3, optionally, the first joining polynucleotide sequence, HR5/HR7, optionally, the second joining polynucleotide sequence, optionally C9, the third selection cassette, optionally C10, and HR6.

Exemplary methods of two rounds of successive recombination or “stitching” are shown above; however, the methods may be performed reiteratively to produce very large constructs. The methods may also be used in parallel, e.g., in arrays, to generate libraries of compositions for successive design, build, test cycles to improve the speed of innovation.

The HR combinations may comprise the same sequence, depending on placement in the vector and the desired result. That is, HR1 and HR3, in the above embodiment, may comprise or consist of the same polynucleotide sequence. Similarly, subsequent rounds of recombination may use the same 3′ HR sequence, e.g., HR2/4/6, may comprise or consist of the same polynucleotide sequence.

### Truncating Off a 3′ Fragment of a Polynucleotide and Joining to a Further Polynucleotide

The method may further include the optional joining sequence to generate a 5′ fragment of the sequence to be truncated joined to the joining sequence. Thus, in some embodiments, the first donor construct comprises, from 5′ to 3′, HR1, the first joining sequence comprising the seventh homologous recombination region (HR7), thereby generating the first recombined polynucleotide comprising, from 5′ to 3′, a fragment of the first sequence to be truncated, HR1, the first joining polynucleotide sequence, the first selection cassette, and HR2/HR4, wherein the first selection cassette is optionally flanked by endonuclease sites.

### Truncating a 5′ Fragment of a Polynucleotide

As discussed above, the disclosed methods may also be used to truncate off a 5′ portion of a sequence to be truncated. However, the scheme is inverted, relative to the 3′ truncation methods, disclosed above (see, e.g., FIGS. 1F and 1G).

Accordingly, in some embodiments, the methods comprise (a) conjugating a first donor cell with a first recipient cell, wherein the first donor cell comprises a first donor construct and wherein the first recipient cell comprises a first recipient polynucleotide, to (i) transfer the first donor construct from the first donor cell to the first recipient cell and (ii) recombine the first donor construct and the first recipient construct in the first recipient cell by homologous recombination, wherein: the first donor construct comprises, from 5′ to 3′, (1) a first endonuclease site (C11), a first sequence to be truncated comprising a first homologous recombination region (HR1), (2) a first selection cassette comprising at least one first selectable marker, (3) a second homologous recombination region (HR2), and (4) a second endonuclease site (C2), wherein HR1 is located at least one bp downstream of the 5′ end of the first sequence to be truncated; the recipient construct comprises, from 5′ to 3′, optionally, a first joining sequence, (1) a third homologous recombination region (HR3) that is homologous to HR1, and (2) a fourth homologous recombination region (HR4) that is homologous to R2, and optionally further comprising a removable selectable construct comprising from 5′ to 3′ an optional third endonuclease site (C3), a second selection cassette comprising at least one second selectable marker, wherein the at least one second selectable marker is distinct from the at least one first selectable marker in the first selection cassette, a fourth endonuclease site (C4); wherein, following the conjugation of the first donor cell and the first recipient cell, the first recipient cell comprises one or more endonuclease specific for C1, C2, and optionally C3, and C4, such that C1, C2, and optionally C3, and C4 are cleaved; thereby providing, following the homologous recombination of HR1 with HR3 and R2 with HR4, a first recombined polynucleotide in the recipient cell comprising, from 5′ to 3′, optionally, the first joining sequence, HR1/HR3, a 3′ fragment of the first sequence to be truncated, the first selection cassette, and R2/HR4.

Thus, because the sequence to be truncated is located in the donor construct, a fragment that is located upstream of the HR1 is removed during homologous recombination, thereby generating only a 3′ fragment of the sequence to be truncated.

As with the other methods disclosed herein, this method may be performed reiteratively, or integrated into a larger scheme combining successive or prior rounds of the different embodiments disclosed herein.

Joining and/or Truncating Polynucleotides Using Bridging Blocks

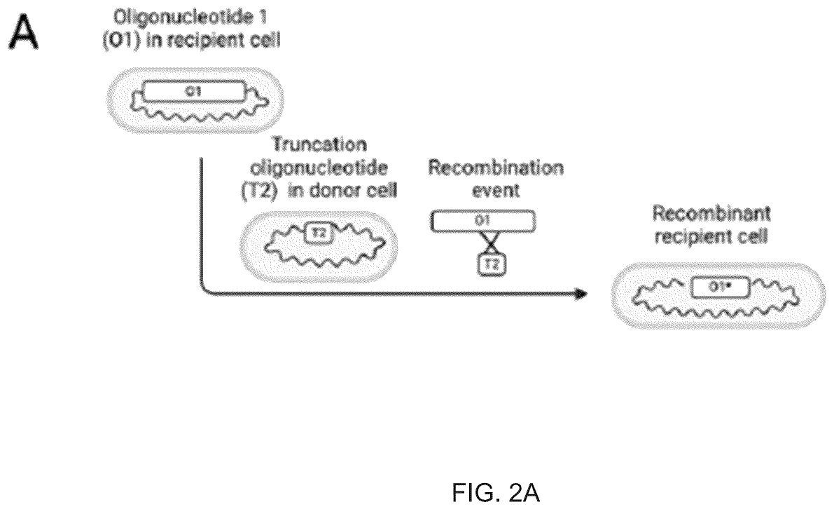

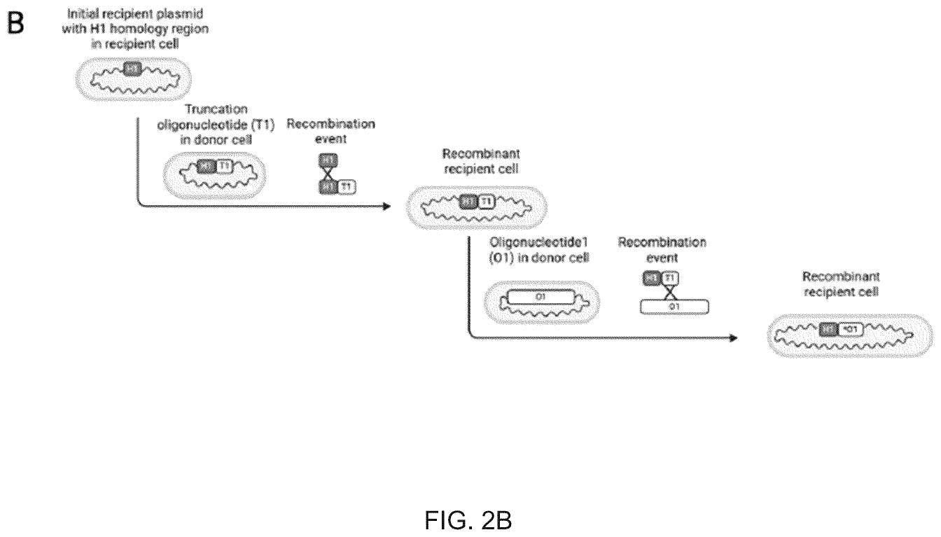

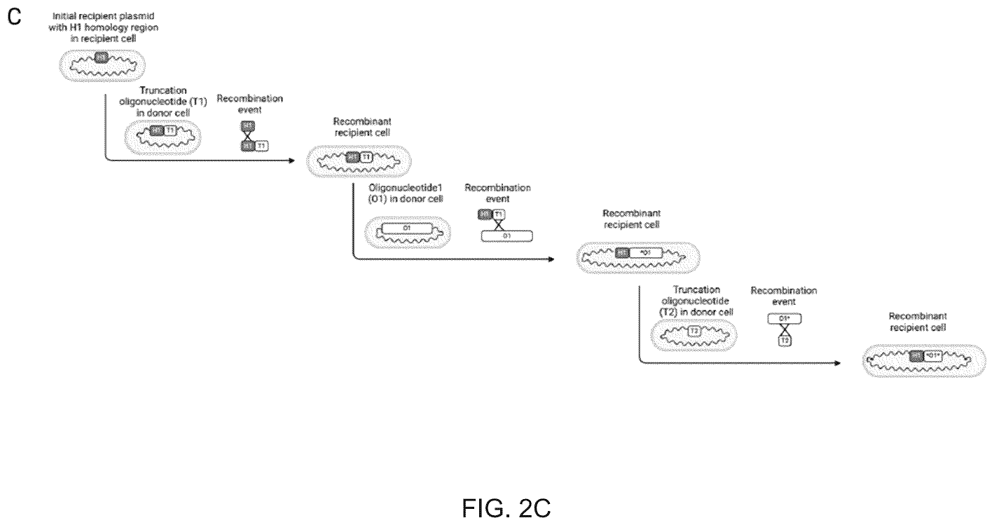

In a further embodiment, “bridging blocks” may be used to (1) scarlessly stitch together DNA elements without homology, (2) truncate sequences of interest, or (3) a combination of (1) and (2). See, e.g., FIGS. 2, 3, 7, and 12. Thus, at least one internally-derived (non-terminal) oligonucleotide region from any one or two, or more, DNA elements (e.g., DNA blocks or DNA assemblies, and the like) already placed in compatible donor plasmids/cells can be seamlessly joined by utilizing two relatively small bridging blocks (e.g., HR regions of 80-100 bp, and the like) that provide homology to both existing blocks (e.g. DNA elements) corresponding to the donor plasmid and recipient oligonucleotides in the invention methods. Accordingly, in a further aspect, methods of joining two polynucleotide sequences are provided. In some embodiments, the methods comprise: (a) conjugating a first donor cell comprising a first bridging construct with a first recipient cell comprising a first recipient polynucleotide to (i) transfer the first bridging construct from the first donor cell to the first recipient cell and (ii) recombine the first bridging construct and the first recipient construct in the first recipient cell by homologous recombination, wherein: the first bridging construct comprises, from 5′ to 3′, (1) a first endonuclease site (C1), (2) a first homologous recombination region (HR1), (3) a fifth homologous recombination region (HR5), (4) a fifth endonuclease site (C5), (5) a first selection cassette comprising at least one selectable marker, (6) a sixth endonuclease site (C6), (7) a second homologous recombination region (HR2), and (8) a second endonuclease site (C2); the recipient construct comprises, from 5′ to 3′, a first joining polynucleotide comprising a third homologous recombination region (HR3); a fourth homologous recombination region (HR4); and optionally further comprising a removable selectable construct comprising a third endonuclease site (C3), a second selection cassette comprising at least one selectable marker, wherein the at least one selectable marker is distinct from the at least one selectable marker in the first selection cassette, a fourth endonuclease site (C4); wherein, following the conjugation of the first donor cell and the first recipient cell, the first recipient cell comprises one or more endonuclease specific for C1, C2, optionally C3, and C4, such that C1, C2, and optionally C3, and C4 are cleaved; thereby providing, following the homologous recombination of HR1 with HR3 and HR2 with HR4, a first recombined polynucleotide in the recipient cell comprising, from 5′ to 3′, HR1/HR3, the first joining polynucleotide, HR5, C5, the first selection cassette, C6, and HR2/HR4; (b) conjugating a second donor cell comprising a first donor construct to a cell comprising the first recombined polynucleotide to (i) transfer the first donor construct from the second donor cell to the cell comprising the first recombined polynucleotide and (ii) recombine the first donor construct and the first recombined polynucleotide in the recipient cell by homologous recombination, wherein: the first donor construct comprises, from 5′ to 3′, (1) a seventh endonuclease site (C7), (2) a second joining polynucleotide sequence comprising a sixth homologous recombination region (HR6) that is homologous to HR5, (3) a ninth endonuclease site (C9), (4) a third selection cassette comprising at least one selectable marker, wherein the at least one selectable marker is distinct from the at least one selectable marker in the first selection cassette, (5) a tenth endonuclease site (C10), (6) a seventh homologous recombination region (HR7), and (7) an eighth endonuclease site (C8); wherein the cell comprising the first recombined polynucleotide comprises at least one endonuclease specific for C5, C6, C7 and C8, such that are C5, C6, C7 and C8 are cleaved; thereby providing, following the homologous recombination of HR6 and HR5 and HR7 and HR2, a second recombined polynucleotide comprising, from 5′ to 3′, HR1, the first joining polynucleotide, HR5/HR6, the second joining polynucleotide, C9, the third selection cassette, C10, and HR2/HR7.

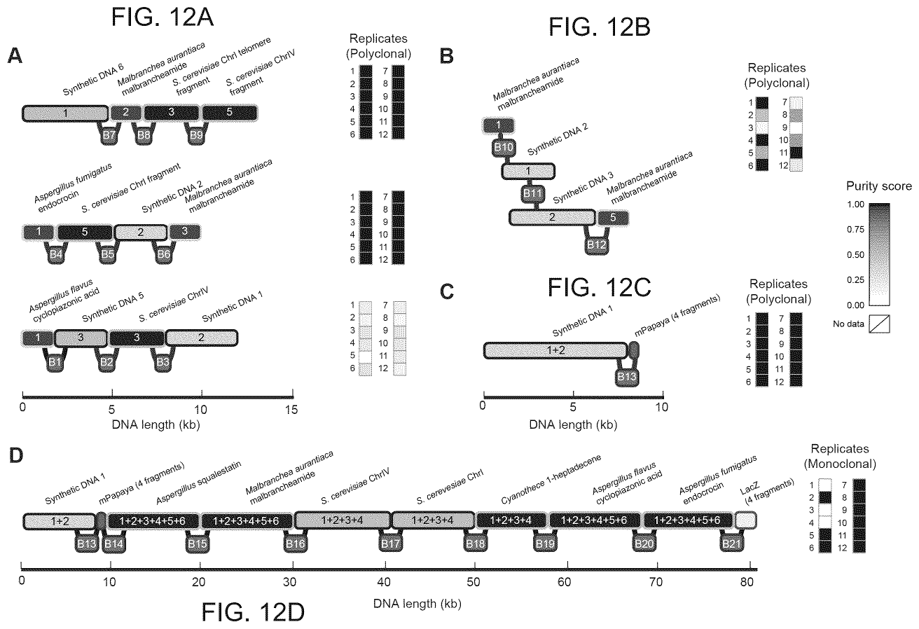

The bridging blocks may comprise a 5′ section comprising an HR homologous to a sequence to be joined at the 5′ end, and a 3′ section that comprises an HR homologous to a sequence to be joined at the 3′ end. In this embodiment, there may be no nucleotides separating the 5′ section and the 3′ section such that there is no junctional scar between the two blocks when assembled. See, e.g., FIG. 12A. Put another way, there may be 10 bp or fewer between the 3′ end of the first joining sequence and the beginning of HR5 in the first bridging construct, or no nucleotides between the 3′ end of the first joining sequence and the beginning of HR5 in the first bridging construct. The two sequences being joined may, in some embodiments, have no regions of homology that is 10 or greater successive nucleotides in length.

Referring now to FIG. 1G, the bridging block may comprise a sequence between HR1 and HR5 that, following recombination, is introduced between JPS1 and JPS2.

The HRs may be designed to truncate an incoming DNA element by designing an HR on the bridging block such that the HR on the bridging block such that the HR begins at least one bp downstream from the incoming (from donor) polynucleotide. See, e.g., FIG. 12B.

Bridging block/oligonucleotide homology regions, for use in the invention methods, can be designed to provide homology to any desired internal location (i.e., non-terminal region) of a partial growing DNA assembly or an existing complete DNA assembly from a recipient cell; or an incoming DNA block from a donor cell, thereby advantageously permitting selected parts of existing DNA blocks to be truncated, and optionally recombined into a growing high throughput DNA assembly without a PCR step. Initial results provided herein indicated that bridges (internal homology regions, such as HR7 in FIG. 1A) can truncate up to 740 bp off of a growing assembly and 500 bp off of an incoming block. The bridging blocks described may be designed to stitch together DNA blocks with no homology by constructing a bridging block having a homologous region (40-50 nucleotides long) with a first DNA to be bridged and a second homologous region (40-50 nucleotides long) with a second DNA to be bridged to allow stitching the two non-related DNA blocks together via the methods provided here.

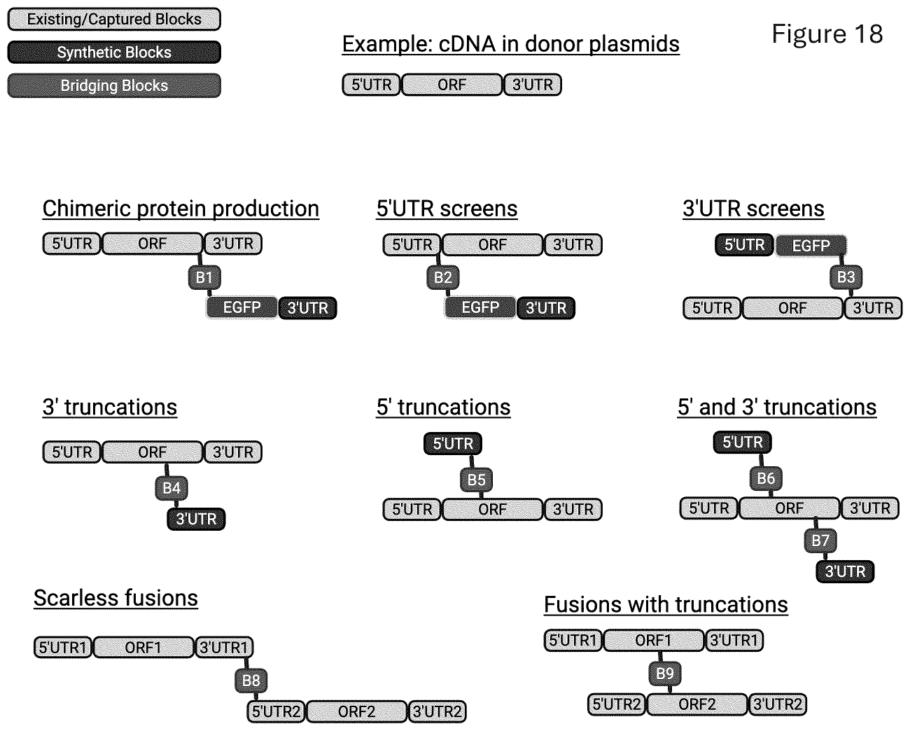

The methods may be useful for several different types of joining or truncating polynucleotides as shown in FIG. 18. The methods may be useful for generating fusion or chimeric proteins comprising distinct N-terminal and C-terminal proteins by stitching together in frame regions of polynucleotides encoding portions of the polypeptides to generate a novel fusion protein. The method could be used to position distinct promoters to drive expression of downstream polynucleotides or to perform 5′ UTR or 3′ UTR screens to assess the effect of these regions on gene expression or RNA stability. The methods could be used to generate libraries of cDNAs, genomic DNAs or splice variants. The methods could be used to generate 3′ truncations, 5′ truncations or both to study protein structure and function as described herein. In addition, scarless gene fusions can be made or fusions of polynucleotides containing truncations can also be generated.

The disclosed methods may be applied to any sequences and is not meant to be limited to any particular sequence. The methods may employ any one of the sequences disclosed herein, e.g., at least one of SEQ ID NOs: 1-19, or sequences with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to at least one of SEQ ID NOs: 1-19.

### RNA-Guided DNA Nucleases

As used herein, the term “RNA-guided DNA endonuclease” refers to any DNA endonuclease that is guided to a target DNA sequence by a helper or guide RNA molecule. Examples of an RNA-guided DNA endonuclease include, but are not limited to, Cas1, Cas1B, Cas2, Cas3, Cas4, Cas5, Cas6, Cas7, Cas8, Cas9 (also known as Csn1 and Csx12), Cas10, Cas12, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4, all variants and homologs thereof.

As used herein, the term “Cas9” or “CRISPR-associated protein 9” is used in accordance with its plain ordinary meaning and refers to an enzyme that uses CRISPR sequences as a guide to recognize and cleave specific strands of DNA that are at least partially complementary to the CRISPR sequence. Cas9 enzymes together with CRISPR sequences form the basis of a technology known as CRISPR-Cas9 that can be used to edit genes within organisms. This editing process has a wide variety of applications including basic biological research, development of biotechnology products, and treatment of diseases.

A “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In aspects, the Cas9 protein is substantially identical to the protein identified by the UniProt reference number Q99ZW2 or a variant or homolog having substantial identity thereto. In aspects, the Cas9 protein has at least 75% sequence identity to the amino acid sequence of the protein identified by the UniProt reference number Q99ZW2. In aspects, the Cas9 protein has at least 80% sequence identity to the amino acid sequence of the protein identified by the UniProt reference number Q99ZW2. In aspects, the Cas9 protein has at least 85% sequence identity to the amino acid sequence of the protein identified by the UniProt reference number Q99ZW2. In aspects, the Cas9 protein has at least 90% sequence identity to the amino acid sequence of the protein identified by the UniProt reference number Q99ZW2. In aspects, the Cas9 protein has at least 95% sequence identity to the amino acid sequence of the protein identified by the UniProt reference number Q99ZW2.

A “CRISPR-associated endonuclease Cas12a,” “Cas12a,” “Cas12” or “Cas12 protein” as referred to herein includes any of the recombinant or naturally-occurring forms of the Cas12 endonuclease or variants or homologs thereof that maintain Cas12 endonuclease enzyme activity (e.g. within at least 50%, 80%, 90%, 95%, 96%, 97%, 98%, 99% or 100% activity compared to Cas12). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas12 protein. In aspects, the Cas12 protein is substantially identical to the protein identified by the UniProt reference number A0Q7Q2 or a variant or homolog having substantial identity thereto.

A “CRISPR-associated endoribonuclease Cas13a,” “Cas13a,” “Cas13” or “Cas13 protein” as referred to herein includes any of the recombinant or naturally-occurring forms of the Cas13 endoribonuclease or variants or homologs thereof that maintain Cas13 endoribonuclease enzyme activity (e.g. within at least 50%, 80%, 90%, 95%, 96%, 97%, 98%, 99% or 100% activity compared to Cas13). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas13 protein. In aspects, the Cas13 protein is substantially identical to the protein identified by the UniProt reference number P0DPB8 or a variant or homolog having substantial identity thereto.

As used herein, “TALEN” or “transcription activator-like effector nuclease” refers to restriction enzymes generated by attaching a DNA binding domain (e.g. a TAL effector DNA-binding domain) to a nuclease (e.g. FokI). TALEN typically includes a naturally occurring DNA-binding domain, which include multiple modules, termed TALs or TALEs. Thus, the TALs, which include variable diresidues, confer DNA binding specificity.

A “guide RNA” or “gRNA” as provided herein refers to an RNA sequence having sufficient complementarity with a target polynucleotide sequence to hybridize with the target sequence and direct sequence-specific binding of a CRISPR complex to the target sequence. For example, a gRNA can direct Cas to the target polynucleotide. In embodiments, the gRNA includes the crRNA and the tracrRNA. For example, the gRNA can include the crRNA and tracrRNA hybridized by base pairing. Thus, in embodiments, the two RNA can be encoded separately by a crRNA and tracrRNA as 2 RNA molecules which then form an RNA/RNA complex due to complementary base pairing between the crRNA and tracrRNA. In aspects, the degree of complementarity between a guide RNA sequence and its corresponding target sequence, when optimally aligned using a suitable alignment algorithm, is about 50%, 60%, 75%, 80%, 85%, 90%, 95%, 97.5%, 99%, or more. In aspects, the degree of complementarity between a guide RNA sequence and its corresponding target sequence, when optimally aligned using a suitable alignment algorithm, is at least about 50%, 60%, 75%, 80%, 85%, 90%, 95%, 97.5%, 99%.

Non-limiting examples of CRISPR enzymes include Cas1, Cas1B, Cas2, Cas3, Cas4, Cas5, Cas6, Cas7, Cas8, Cas9 (also known as Csn1 and Csx12), Cas10, Cas12, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In embodiments, the CRISPR enzyme is a Cas9 enzyme. In embodiments, the Cas9 enzyme is S. pneumoniae, S. pyogenes or S. thermophilus Cas9, or mutants derived thereof in these organisms. In embodiments, the CRISPR enzyme is codon-optimized for expression in a eukaryotic cell. In embodiments, the CRISPR enzyme directs cleavage of one or two strands at the location of the target sequence. In embodiments, the CRISPR enzyme lacks DNA strand cleavage activity.

As used herein, a “zinc finger” is a polypeptide structural motif folded around a bound zinc cation. In embodiments, the polypeptide of a zinc finger has a sequence of the form X₃-Cys-X₂₋₄-Cys-X₁₂-His-X₃₋₅-His-X₄, wherein X is any amino acid (e.g., X₂₋₄ indicates an oligopeptide 2-4 amino acids in length). Thus, “zinc finger nuclease” as used herein refers to a nuclease including a zinc finger motif and a domain capable of inducing breaks in the target DNA.

In another aspect of the invention method, step (b) is repeated for one or more iterations with a third or subsequent donor cell comprising a third or subsequent donor plasmid comprising compatible HR regions and a third or subsequent oligonucleotide comprising a fourth or subsequent DNA element (oligo4, oligo5, . . . oligoN), thereby forming a third or subsequent recombined-recipient oligonucleotide comprising at least a portion of the first, the second, third, fourth and/or subsequent DNA elements which together form a third or subsequent recombined-recipient oligonucleotide.

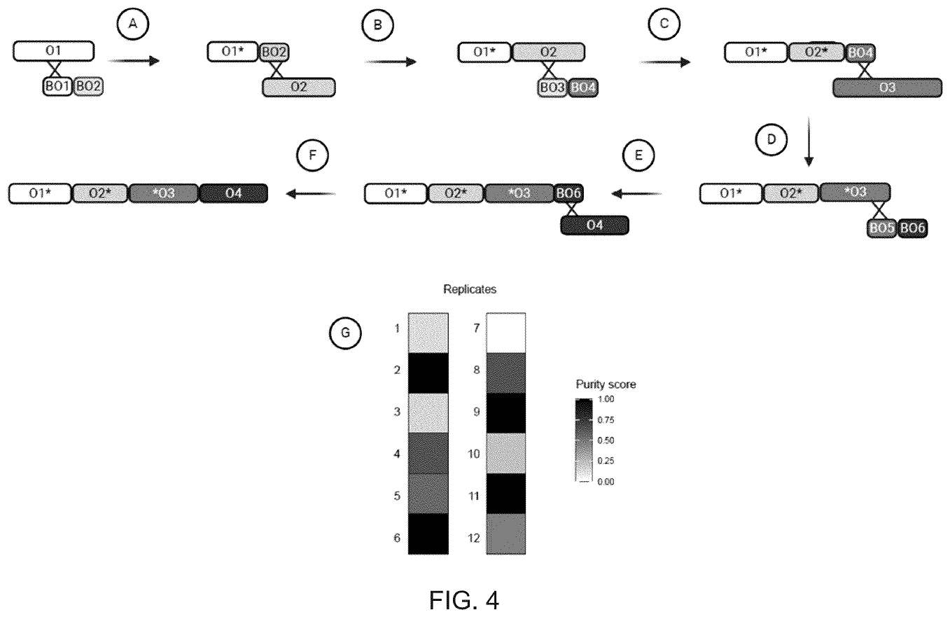

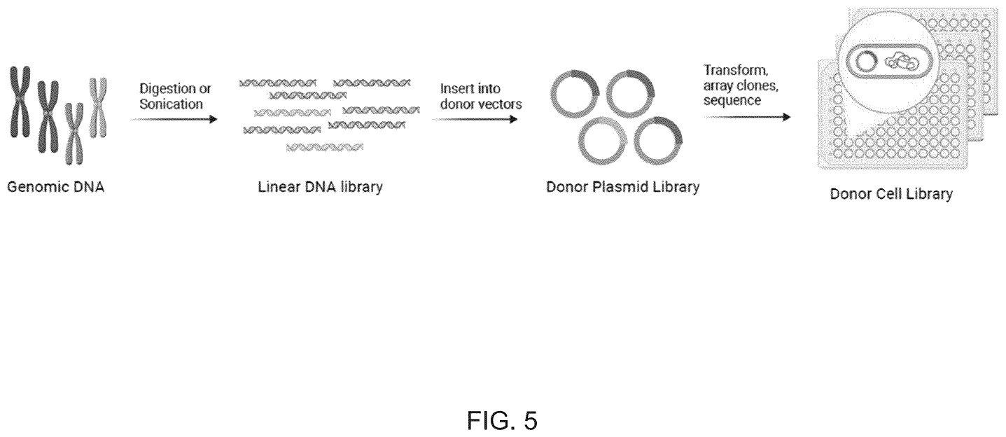

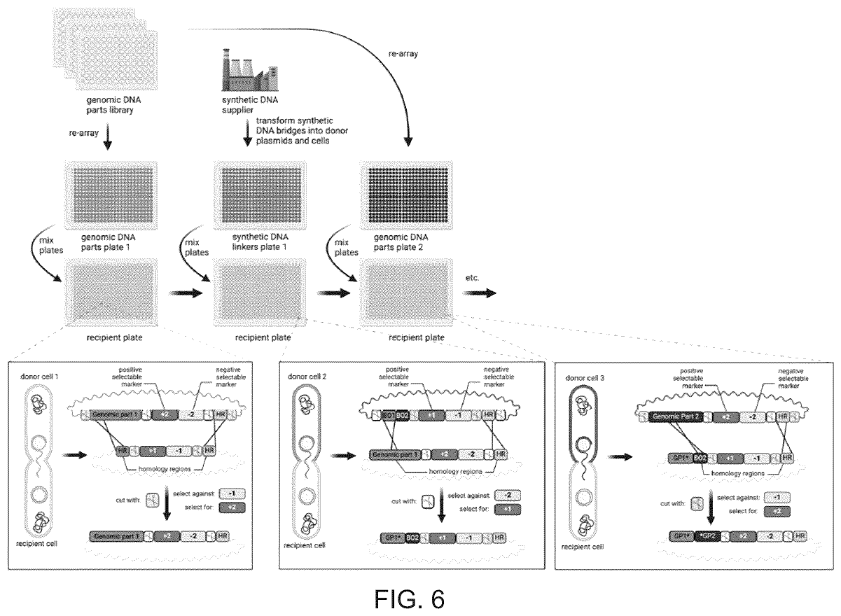

The term, “composability,” “composable,” or grammatical variations thereof, refers to the ability to reuse and repurpose code snippets, e.g., genetic code snippets, with low operational friction. Composability has been a critical feature of information programming platforms because it accelerates the ability of engineers to build on top of each other's work. Repurposing of DNA blocks using existing in vitro methods generally requires that they be amplified with long primers by PCR to produce compatible ends, purified, validated, re-assembled, and transformed back into cells, a process that is difficult to automate and scale. By contrast, in accordance with the present invention, at least one internally-derived (non-terminal) oligonucleotide region from any one or two, or more, DNA elements (e.g., DNA blocks or DNA assemblies, and the like) already placed in compatible donor plasmids/cells can be seamlessly joined by utilizing two relatively small bridging blocks (e.g., HR regions of 80-100 bp, and the like) that provide homology to both existing blocks (e.g. DNA elements) corresponding to the donor plasmid and recipient oligonucleotides in the invention methods (such as particular homology regions as set forth in FIGS. 1-7, and the like).

Multiple DNA synthesis providers have scaled processes to offer DNA blocks (e.g., recipient oligonucleotides or DNA elements) already cloned into a vector of choice. In accordance with the present invention, the skilled artisan can readily adapt these existing processes and existing DNA blocks to function with the invention donor plasmids, cells, and methods, such that the skilled artisan can readily obtain “stitch-ready” DNA blocks in donor plasmids/cells, permitting mid- to high-throughput DNA stitching assembly by mating cells according the invention methods.

Accordingly, also contemplated herein is the construction of arrayed donor cell libraries that create a reservoir of useful DNA sequences (DNA blocks or DNA elements) that can be recombined into a growing assembly without PCR (e.g. a human genome BAC or YAC library, and the like). The composability provided by the invention methods, advantageously permits DNA block storage, reuse, and sharing, facilitating the development of a robust disaggregated DNA engineering ecosystem where skilled artisans can build on top of each other's designs.

This “code base” of DNA constructs permits those of skill in the art to source DNA constructs from various repositories and incorporate the desired fragment from each construct into a recombineered designer DNA assembly. In certain embodiments, some existing DNA fragments from repositories can be sourced and combined with other DNA oligonucleotides that have been synthesized de novo. The advantages and rationale for using the invention methods for sourcing DNA fragments/oligonucleotides from repositories rather than synthesize them de novo include: 1) the DNA fragment can not be synthesized de novo because of certain sequence features that are difficult to synthesize (e.g. tandem repeats, interspersed repeats, high GC content, DNA structure); 2) the DNA fragment is more expensive to synthesize de novo; 3) the DNA fragment takes a longer amount of time to synthesize de novo; and 4) the confidentiality of the desired DNA fragment can be maintained versus disclosing it to a DNA synthesis provider.

### Definitions and Related Embodiments

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” means that the value may vary by +/−10%.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

As may be used herein, the terms “nucleic acid,” “nucleic acid molecule,” “nucleic acid sequence,” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, sgRNA, guide RNA, tracrRNA, a recombinant polynucleotide, a branched polynucleotide, a plasmid, a vector, isolated DNA of a sequence, isolated RNA of a sequence, a PCR product, a nucleic acid probe, and a primer. Polynucleotides useful in the methods of the disclosure may comprise natural nucleic acid sequences and variants thereof, artificial nucleic acid sequences, or a combination of such sequences.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides, including “bridging-oligonucleotides” as used herein. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides, contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids including known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids including one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, the phrase “DNA assembly” or grammatical variants thereof, refers to a recombined-recipient oligonucleotide formed by stitching or joining via recombination using in vivo bacterial conjugation, at least two oligonucleotides (e.g., DNA elements) to form a recombined-recipient oligonucleotide within a recipient cell.

The phrase “bridging-blocks”, or “HR-regions”, or grammatical variations thereof, refers to small homologous recombination regions (e.g., HR5, HR2) that in particular embodiments provide homology to two sub-regions of DNA elements that are being stitched together via in vivo recombination (see, e.g., FIG. 1G). In other embodiments, a bridging block/HR-region corresponds to a single region of homology to an internal region of a recipient oligonucleotide, such that the recipient oligonucleotide is truncated upon the recombination event (see, e.g., FIG. 2, panel A, block T2). The bridging blocks/HR-regions, for use herein, in certain embodiments can be from 20-1000 or more nucleotides in length. In other embodiments, the bridging blocks/HR-regions can be ranges selected from the group consisting of: from 30-900, from 40-800, from 50-700, from 50-600, from 50-500, from 50-400, from 50-300, from 50-200, from 60-150, from 70-125, and/or from 80-100 nucleotides in length.

A “barcode” refers to one or more nucleotide sequences that are used to identify a cell or a plurality of cells with which the barcode is associated. Barcodes can be 3-1000 or more nucleotides in length, preferably 3-250 nucleotides in length, and more preferably 4-40 nucleotides in length, including any length within these ranges, such as 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, or 1000 nucleotides in length. A barcode is “unique” when the barcode is (statistically) present in about one cell in a population of cells. The cell containing the barcode can then be expanded to make a clonal plurality of cells, such that each cell of the plurality of cells contains the same barcode. For example, “a plurality of barcoded cells, wherein each barcoded cell comprises a single, unique barcode” may refer to a population of cells which contains (statistically) a single cell containing a given barcode or a unique combination of barcodes. Alternatively, it may refer to a population of cells which contains a plurality of clonal populations of cells, each cell of each clonal population containing the same barcode, but cells of different clonal populations containing different barcodes.

As used herein, the term “complement,” refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

As used herein, the term “gene” is used in accordance with its plain ordinary meaning and refers to the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The term “expression vector” refers to a nucleic acid molecule that encodes for genes and/or regulatory elements necessary for the expression of genes. Expression of a gene from a vector, which may in the form of a plasmid, can occur in cis or in trans. If a gene is expressed in cis, the gene and regulatory elements are encoded by the same plasmid. Expression in trans refers to the instance where the gene and the regulatory elements are encoded by separate plasmids.

As used herein, the term “vector” refers to a nucleic acid molecule capable of transporting another nucleic acid to which it has been linked. A vector may be in the form of a “plasmid”, which in this context refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cell type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, including the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The term “promoter” as used herein refers to a region of DNA that initiates transcription of a particular gene. Promoters are typically located near the transcription start site of a gene, upstream of the gene and on the same strand (i.e., 5′ on the sense strand) on the DNA. Promoters may be, e.g., about 100 to about 1000 base pairs in length.

A nucleotide base “position” is denoted by a number that sequentially identifies each amino acid (or nucleotide base) in the reference sequence based on its position relative to the 5′-end. Due to deletions, insertions, truncations, fusions, and the like that must be taken into account when determining an optimal alignment, in general the amino acid residue number in a test sequence determined by simply counting from the 5′-end will not necessarily be the same as the number of its corresponding position in the reference sequence. For example, in a case where a variant has a deletion relative to an aligned reference sequence, there will be no nucleotide base in the variant that corresponds to a position in the reference sequence at the site of deletion. Where there is an insertion in an aligned reference sequence, that insertion will not correspond to a numbered nucleotide position in the reference sequence. In the case of truncations or fusions there can be stretches of nucleotides in either the reference or aligned sequence that do not correspond to any nucleotide in the corresponding sequence.

The terms “numbered with reference to” or “corresponding to,” when used in the context of the numbering of a given polynucleotide sequence, refers to the numbering of the residues of a specified reference sequence when the given polynucleotide sequence is compared to the reference sequence.

As used herein, the term “virus” or “virus particle” is used according to its plain ordinary meaning within the context of viral transduction. Transduction with viral vectors can be used to insert or modify genes in mammalian cells.

As used herein, the terms “genetic modification”, “gene modification”, “gene editing”, “genetic editing”, “genome editing”, “genome engineering” or the like refer to a type of genetic engineering in which DNA is inserted, deleted, modified or replaced at one or more specified locations in the genome of a cell. One key step in gene editing is creating a double stranded break at a specific point within a gene or genome. Examples of gene editing tools such as nucleases that accomplish this step include but are not limited to Zinc finger nucleases (ZFNs), transcription activator like effector nucleases (TALEN), meganucleases, and clustered regularly interspaced short palindromic repeats system (CRISPR/Cas).

As used herein, “DNA element” “DNA elements” or grammatical variations thereof, refer to any DNA sequence that can be transferred between cells, such as between a donor cell and a recipient cell. Thus, a DNA element includes, but is not limited to genomic DNA, a gene, a promoter, an enhancer, a terminator, an intron, an intergenic region, a barcode, or a gRNA. A DNA element may be a fragment of a gene, a promoter, an enhancer, a terminator, an intron, an intergenic region, a barcode, or a gRNA. A DNA element may be a combination of genes, promoters, enhancers, terminators, introns, intergenic regions, barcodes, gRNAs, and fragments of a genes, promoters, enhancers, terminators, introns, intergenic regions, barcodes, and gRNAs. The DNA element, as used herein, can be a recombined-recipient oligonucleotide produced herein. In embodiments, the DNA element is in a donor plasmid. In other embodiments, the DNA element is moved to or is in a recipient oligonucleotide. In other embodiments, the DNA element is moved from a recipient oligonucleotide to a reset donor plasmid.

As used herein, the term “gene editing reagent” refers to components required for gene editing tools and may include enzymes, riboproteins, solutions, co-factors and the like. For example, gene editing reagents include one or more components required for Zinc finger nucleases (ZFNs), transcription activator like effector nucleases (TALEN), meganucleases, and clustered regularly interspaced short palindromic repeats system (CRISPRICas) gene editing.

As used herein, the term “endonuclease” refers to an enzyme or a component of an endonuclease system (e.g., any component of CRISPR, including a gRNA) which possesses endonucleolytic catalytic activity for polynucleotide cleavage. For example, an endonuclease or component thereof can cleave a phosphodiester bond of an oligonucleotide or polynucleotide. An endonuclease cleaves at a phosphodiester bond within or adjacent to its recognition site sequence, which spans at least 4 bp in length. Types of endonucleases include, but are not limited to restriction enzymes, AP endonuclease, T7 endonuclease, T4 endonuclease, Bal 31 endonuclease, Endonuclease I, Micrococcal nuclease, Endonuclease II, Neurospora endonuclease, S1 endonuclease, PI-nuclease, Mung bean nuclease I, DNAse I, RNA-guided DNA endonuclease, (e.g. CRISPR, including any CRISPR components, e.g. Cas protein, gRNA, etc.), Homothallic switching endonuclease, TALENs, zinc finger nucleases, and Endo R.

By “cleavage” it is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In some embodiments, a complex including a guide RNA and a site-specific modifying enzyme is used for targeted double-stranded DNA cleavage.

As used herein, the term “CRISPR” or “clustered regularly interspaced short palindromic repeats” is used in accordance with its plain ordinary meaning and refers to a genetic element that bacteria use as a type of acquired immunity to protect against viruses. CRISPR includes short sequences that originate from viral genomes and have been incorporated into the bacterial genome. Cas (CRISPR associated proteins) process these sequences and cut matching viral DNA sequences. Thus, CRISPR sequences function as a guide for Cas to recognize and cleave DNA that are at least partially complementary to the CRISPR sequence. By introducing plasmids including Cas genes and specifically constructed CRISPRs into eukaryotic cells, the eukaryotic genome can be cut at any desired position.

The term “homologous recombination” refers to a type of genetic recombination where information is exchanged between two similar or identical nucleic acid sequences, which may be referred to herein as “homology regions”. In some embodiments of the methods described here, a homology region may comprise, for example, two areas of homology which may optionally flank a non-homologous region. In embodiments of the methods described here, an E. coli RecA gene may be used for boosting homologous recombination. “RecA” refers to the bacterial homolog of the family of ubiquitous 38-kD homologous DNA repair proteins which mediates ATP-dependent homologous recombination in bacteria. In embodiments, the donor cell or recipient cell of the methods described herein includes an oligonucleotide encoding one or more homologous DNA repair genes, such as RecA. In embodiments, homologous DNA repair gene expression is inducible. In embodiments, the homologous DNA repair gene is RecA. In embodiments, homologous DNA repair genes are the recombineering genes Redα, Redβ, and Redγ. Non-limiting examples of methods for homologous recombination and gene editing using various nuclease systems can be found, for example, in U.S. Pat. No. 8,945,839, International PCT application Pub. No. WO2013/163394 and U.S. Patent Application Nos. 2016/0060657, 2012/0192298A1 and US2007/0042462. These and other known methods for homologous recombination can be used in combination with the methods described here.

As used herein, the term “transfection” is used in accordance with its plain ordinary meaning and refers to a process of deliberately introducing naked or purified nucleic acids into eukaryotic cells. In instances, “transfection” may refer to other methods and cell types, although other terms are often preferred. For example, the term “transformation” is typically used to describe non-viral DNA transfer in bacteria and non-animal eukaryotic cells, including plant cells. In animal cells, transfection is the preferred term. For example, the term “transduction” is often used to describe virus-mediated gene transfer into eukaryotic cells.

The terms “bacterial conjugation” and “bacterial mating” are interchangeable and refer to a mode of genetic exchange between bacteria. Typically, the bacterial conjugation involves only a portion of the genome of one of the cells (the donor) and the complete genome of its partner (the recipient cell). Thus, genetic transfer in bacterial conjugation is typically partial. In embodiments, bacterial conjugation is transfer of non-genomic bacterial DNA from a donor cell to a recipient cell. In instances, bacterial conjugation occurs through a plasmid. In instances, bacterial conjugation occurs through an exogenous DNA in the bacteria. In embodiments, the donor cell and the recipient cell are in contact for bacterial conjugation to occur. In embodiments, the donor cell and the recipient cell include linking bridge (e.g. pilus) for bacterial conjugation to occur.

In embodiments, the recipient cell or the donor cell includes an oligonucleotide that enables plasmid conjugation. In embodiments, the oligonucleotide that enables plasmid conjugation is in the donor cell genome. In embodiments, the oligonucleotide that enables plasmid conjugation is in a helper plasmid. In embodiments, the oligonucleotide that enables plasmid conjugation is the Tra operon. In embodiments, the oligonucleotide that enables plasmid conjugation is selected from: IncF1 Tra (traA, traB, traC, traD, traE, traF, traG, traH, traI, traJ, traK, traL, traM, traN, traO, traP, traQ, traR, traS, traT), IncP Tra operon: (trbA, trbB, trbC, trbD, trbE, trbF, trbG, trbH, trbI, trbJ, trbK, trbL, traA, traB, traC, traD, traE, traF, traG, traH, traI, traJ, traK, traL, traM, traN, traO), IncI1 tra operon: (traE, traF, traG, traH, traI, traJ, traK, traL, traM, traN, traO, traP, traQ, traS, traT, traU, traV, traW, traY), pTiC58 tra genes: (traA, traF, traB, traC, traG, traD, traR, traI), and pIJ101: clt, korB. In embodiments, the oligonucleotide that enables plasmid conjugation is IncF1 Tra (traA, traB, traC, traD, traE, traF, traG, traH, traI, traJ, traK, traL, traM, traN, traO, traP, traQ, traR, traS, traT). In embodiments, the oligonucleotide that enables plasmid conjugation is IncP Tra operon: (trbA, trbB, trbC, trbD, trbE, trbF, trbG, trbH, trbI, trbJ, trbK, trbL, traA, traB, traC, traD, traE, traF, traG, traH, traI, traJ, traK, traL, traM, traN, traO). In embodiments, the oligonucleotide that enables plasmid conjugation is IncI1 tra operon: (traE, traF, traG, traH, traI, traJ, traK, traL, traM, traN, traO, traP, traQ, traS, traT, traU, traV, traW, traY). In embodiments, the oligonucleotide that enables plasmid conjugation is pTiC58 tra genes: (traA, traF, traB, traC, traG, traD, traR, traI). In embodiments, the oligonucleotide that enables plasmid conjugation is pIJ101: clt, korB.

As used herein, “donor cell” refers to a cell (e.g. a bacteria cell) that transfers genetic material to another cell (e.g. a bacteria cell, a plant cell, etc.). The cell that receives the transferred genetic material is referred to herein as a “recipient cell”.

The term “donor plasmid”, as used herein refers to DNA from a donor cell (e.g. a bacteria cell) including an oligonucleotide sequence (e.g. donor DNA, oligonucleotide including a DNA element) that is to be transferred from the donor cell to a recipient cell (e.g. a bacteria cell, yeast cell, plant cell, etc.). Typically, the donor plasmid is a circular double-stranded DNA that is separate from genomic DNA. Thus, the term “recipient plasmid” refers to DNA from a recipient cell that receives the donor DNA. In embodiments, the DNA from the donor plasmid is received by DNA other than DNA from a recipient plasmid. Thus, in embodiments, the donor DNA may be incorporated into genomic DNA.

In embodiments, the donor plasmid includes an origin of transfer. In embodiments, the origin of transfer is from a mobile element. In embodiments, the mobile element is a plasmid. In embodiments, the plasmid is an IncFI plasmid, an IncPa plasmid, an IncI1 plasmid, a pTiC58 from Agrobacterium tumefaciens, a pAD1 plasmid, an Inc18 plasmid, or an IncH plasmid. In embodiments, the plasmid is an IncFI plasmid. In embodiments, the plasmid is an IncPa plasmid. In embodiments, the plasmid is an IncI1 plasmid. In embodiments, the plasmid is a pTiC58 from Agrobacterium tumefacien. In embodiments, the plasmid is a pAD1 plasmid. In embodiments, the plasmid is an Inc18 plasmid. In embodiments, the plasmid is an IncH plasmid. The plasmids are discussed in greater detail in Ippen-Ihler, K. A., and Minkley, E. G., Jr., (1986). The conjugation system of F, the fertility factor of Escherichia coli. Ann. Rev. Genet. 20:593-624.; Guiney, D. G., and Lanka, E., (1989), Conjugative transfer of IncP plasmids, in: Promiscuous Plasmids of Gram-negative Bacteria (C. M. Thomas, ed.), Academic Press, London, pp. 27-56.; Catherine E. D. Rees, David E. Bradley, Brian M. Wilkins, (1987) Organization and regulation of the conjugation genes of IncI1 plasmid ColIb-P9. Plasmid. 18: 223-236.; von Bodman S B, McCutchan J E, Farrand S K. (1989) Characterization of conjugal transfer functions of Agrobacterium tumefaciens Ti plasmid pTiC58. J. Bacteriol. 171(10):5281-5289.; Clewell D B, Weaver K E. (1989) Sex pheromones and plasmid transfer in Enterococcus faecalis. Plasmid. 21(3):175-84.; Kohler V, Vaishampayan A, Grohmann E. (2018) Broad-host-range Inc18 plasmids: Occurrence, spread and transfer mechanisms. Plasmid. 99:11-21.; Andreas Schlüter, Patrice Nordmann, Remy A. Bonnin, Yves Millemann, Felix G. Eikmeyer, Daniel Wibberg, Alfred Pühler, Laurent Poirel. (2014) IncH-Type Plasmid Harboring bla_{CTX-M-15}, bla_{DHA-1}, and qnrB4 Genes Recovered from Animal Isolates. Antimicrobial Agents and Chemotherapy 58(7):3768-3773. The entire contents of these references are incorporated herein by reference in their entirety for all purposes.

In embodiments, the origin of transfer is from a mobile element. In embodiments, the mobile element is from a conjugative transposon. In embodiments, the conjugative transposon is Tn916 from Enterococcus faecalis or CTnDOT from Bacteroides. In embodiments, the conjugative transposon is Tn916 from Enterococcus faecalis. In embodiments, the conjugative transposon is CTnDOT from Bacteroides. In embodiments, the mobile element is from an integrating conjugative element. In embodiments, the mobile element is from SXT from Vibrio cholerae or R391 from Providencia rettgeri. In embodiments, the mobile element is from SXT from Vibrio cholerae. In embodiments, the mobile element is from R391 from Providencia rettgeri. The elements are described in more detail in references Rice L. B. (1998). Tn916 family conjugative transposons and dissemination of antimicrobial resistance determinants. Antimicrobial agents and chemotherapy, 42(8), 1871-1877.; Cheng Q, Paszkiet B J, Shoemaker N B, Gardner J F, Salyers A A. (2000) Integration and excision of a Bacteroides conjugative transposon, CTnDOT. J Bacteriol. 182(14):4035-43.; Bianca Hochhut and Matthew K. Waldor. (1999) Site-specific integration of the conjugal Vibrio cholerae SXT element into prfC. Mol. Microbiology. 32(1):99-110.; Boltner D, MacMahon C, Pembroke J T, Strike P, Osborn A M. R391: a conjugative integrating mosaic comprised of phage, plasmid, and transposon elements. J Bacteriol. 2002; 184(18):5158-5169., each of which is herein incorporated by reference in its entirety.

In embodiments, the donor plasmid includes a conditional replication origin. In embodiments, the conditional replicon is R6K-pir, RSF1010 oriV—RepA/B/C, ColE2 P9-RepA, RP4 oriV-trfA, pPS10 oriV-RepA, pSC101 ori—RepC^{TS}, RK2 oriV, bacteriophage P1 ori, plasmid pSC101 origin of replication, bacteriophage lambda ori, pBR322 plasmid, pSU739 plasmid, or pSU300 plasmid. In embodiments, the conditional replicon is R6K-pir. In embodiments, the conditional replicon is RSF1010 oriV—RepA/B/C. In embodiments, the conditional replicon is ColE2 P9—RepA. In embodiments, the conditional replicon is RP4 oriV-trfA. In embodiments, the conditional replicon is pPS10 oriV-RepA. In embodiments, the conditional replicon is pSC101 ori—RepC^{TS}. In embodiments, the conditional replicon is RK2 oriV. In embodiments, the conditional replicon is bacteriophage P1 ori. In embodiments, the conditional replicon is plasmid pSC101 origin of replication. In embodiments, the conditional replicon is bacteriophage lambda ori. In embodiments, the conditional replicon is pBR322 plasmid. In embodiments, the conditional replicon is pSU739 plasmid. In embodiments, the conditional replicon is pSU300 plasmid. The plasmids are described in references: Metcalf W W, Jiang W, Daniels L L, Kim S K, Haldimann A, Wanner B L. (1996) Conditionally replicative and conjugative plasmids carrying lacZ alpha for cloning, mutagenesis, and allele replacement in bacteria. Plasmid. 35(1):1-13.; Scherzinger E, Bagdasarian M M, Scholz P, Lurz R, Rückert B, Bagdasarian M. (1984) Replication of the broad host range plasmid RSF1010: requirement for three plasmid-encoded proteins. Proc Natl Acad Sci USA. 81(3):654-8.; ColE2-P9: Yagura M, Nishio S Y, Kurozumi H, Wang C F, Itoh T. (2006) Anatomy of the replication origin of plasmid ColE2-P9. J Bacteriol. 188(3):999-1010.; Ayres E K, Thomson V J, Merino G, Balderes D, Figurski D H. Precise deletions in large bacterial genomes by vector-mediated excision (VEX). (1993) The trfA gene of promiscuous plasmid RK2 is essential for replication in several gram-negative hosts. J Mol Biol. 5; 230(1):174-85.; Maestro B, Sanz J M, Diaz-Orejas R, Fernindez-Tresguerres E. (2003) Modulation of pPS10 host range by plasmid-encoded RepA initiator protein. J Bacteriol. 185(4):1367-75.; Hashimoto-Gotoh, T., & Sekiguchi, M. (1977). Mutations of temperature sensitivity in R plasmid pSC101. Journal of bacteriology, 131(2), 405-412.; Ayres E K, Thomson V J, Merino G, Balderes D, Figurski D H. Precise deletions in large bacterial genomes by vector-mediated excision (VEX). (1993) The trfA gene of promiscuous plasmid RK2 is essential for replication in several gram-negative hosts. J Mol Biol. 5; 230(1):174-85. Stenzel T T, Patel P, Bastia D. (1987) The integration host factor of Escherichia coli binds to bent DNA at the origin of replication of the plasmid pSC101. Cell. 5; 49(5):709-17.; Sugiura S, Ohkubo S, Yamaguchi K. (1993) Minimal essential origin of plasmid pSC101 replication: requirement of a region downstream of iterons. J Bacteriol. 175(18):5993-6001.; Pal S K, Mason R J, Chattoraj D K. (1986) P1 plasmid replication. Role of initiator titration in copy number control. J Mol Biol. 20; 192(2):275-85.; LeBowitz J H, McMacken R. (1984) The bacteriophage lambda O and P protein initiators promote the replication of single-stranded DNA. Nucleic Acids Res. 12(7):3069-3088.; Grindley N D, Kelley W S. (1976) Effects of different alleles of the E. coli K12 pol A gene on the replication of non-transferring plasmids. Mol Gen Genet. 2; 143(3):311-8.; Francia, M. V., & Garcia Lobo, J. M. (1996). Gene integration in the Escherichia coli chromosome mediated by Tn21 integrase (Int21). Journal of bacteriology, 178(3), 894-898.; Mendiola M V, de la Cruz F. (1989) Specificity of insertion of IS91, an insertion sequence present in alpha-haemolysin plasmids of Escherichia coli. Mol Microbiol. 3(7):979-84. The references are incorporated herein in their entirety.

In embodiments, the conditional replication origin is dependent on presence of an oligonucleotide. In embodiments, the oligonucleotide encodes pir1, pir1-116, repA/repB/repC (RSF1010 replicon), repA (ColE2-P9 replicon), trfA (RP4 replicon), RepA (pSP10 replicon), RepC^{TS} (pSC101 replicon), or a combination thereof. In embodiments, the oligonucleotide encodes pir1. In embodiments, the oligonucleotide encodes pir1-116. In embodiments, the oligonucleotide encodes repA/repB/repC (RSF1010 replicon). In embodiments, the oligonucleotide encodes repA (ColE2-P9 replicon). In embodiments, the oligonucleotide encodes trfA (RP4 replicon). In embodiments, the oligonucleotide encodes RepA (pSP10 replicon). In embodiments, the oligonucleotide encodes RepC^{TS} (pSC101 replicon).

In embodiments, the conditional replication origin depends on a condition of cell growth. In embodiments, the condition is temperature.

For the methods provided herein, in embodiments, the donor plasmid or recipient oligonucleotide includes a replicon that can replicate plasmids of lengths from 20 or 30 kilobases.

For the methods provided herein, in embodiments, the donor plasmid or recipient oligonucleotide includes a replicon that that can replicate plasmids of lengths greater than 30 kilobases. In embodiments, the replicon can replicate plasmids of lengths of about 30 kilobases to about 500 kilobases. In embodiments, the replicon can replicate plasmids of lengths of about 30, 50, 70, 90, 100, 120, 140, 160, 180, 200, 220, 240, 260, 280, 300, 350, 400, 450, or 500 kilobases. The length may be any value or subrange within the indicated ranges, including endpoints.

In embodiments, the replicon is from a P1-derived artificial chromosome or a bacterial artificial chromosome. In embodiments, the replicon is from a P1-derived artificial chromosome. In embodiments, the replicon is from a bacterial artificial chromosome. In embodiments, the donor plasmid or recipient oligonucleotide includes an inducible high-copy replication of origin. In embodiments, the donor plasmid includes an inducible high-copy replication of origin. In embodiments, the recipient oligonucleotide includes an inducible high-copy replication of origin.

In embodiments, the donor plasmid or recipient oligonucleotide is a yeast artificial chromosome (YAC), a bacterial artificial chromosome (BAC), a mammalian artificial chromosome (MAC), a human artificial chromosome (HAC), or a plant artificial chromosome (PAC). In embodiments, the donor plasmid is a yeast artificial chromosome (YAC). In embodiments, the donor plasmid is a mammalian artificial chromosome (MAC). In embodiments, the donor plasmid is a human artificial chromosome (HAC). In embodiments, the donor plasmid is a plant artificial chromosome. In embodiments, the recipient oligonucleotide is a yeast artificial chromosome (YAC). In embodiments, the recipient oligonucleotide is a mammalian artificial chromosome (MAC). In embodiments, the recipient oligonucleotide is a human artificial chromosome (HAC). In embodiments, the recipient oligonucleotide is a plant artificial chromosome.

As used herein, the phrase “captured genomic DNA or cDNA” refers to genomic DNA or cDNA (e.g., 50-500 kb are contemplated) stored on plasmids (e.g., donor plasmids). The captured genomic or cDNA can be that within an organized DNA library in cells, such YAC, BAC, MAC, HAC, PAC libraries, and the like.

In embodiments, the donor plasmid or recipient oligonucleotide comprises a conjugation competent vector, which may be a viral vector. In embodiments, the donor plasmid is a viral vector. In embodiments, the recipient oligonucleotide is a viral vector. In embodiments, the viral vector is a retrovirus. In embodiments, the viral vector is a lentivirus. In embodiments, the viral vector is an adenovirus. In embodiments, the viral vector is an adeno-associated virus. In embodiments, the viral vector is a tobacco mosaic virus. In embodiments, the viral vector is a baculovirus. In embodiments, the viral vector is a herpes simplex virus. In embodiments, the viral vector is a poxvirus. In embodiments, the viral vector is gammaretrovirus. In embodiments, the viral vector is Sendai virus.

As used herein, the term “control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

As used herein, the term “contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

As used herein, the terms “origin of transfer” or “oriT” refer to a short sequence (up to 500 bp) that is necessary for transfer of DNA from a bacterial host and recipient during bacterial conjugation.

As used herein, “curable origin of replication” refers to an origin of replication that does not replicate when cells are grown in the presence of certain chemicals or environmental conditions. Under these conditions, plasmids containing a curable origin of replication are lost from the cell. For example, pSC101 ori^{TS} does not function at high temperature and is lost.

As used herein, the term “mobile element” is a type of genetic material that can move around within a genome, or can be transferred between genomes, even between species.

As used herein, the term “conjugative transposon” refers to integrated DNA elements that excise themselves to form a covalently closed circular intermediate that can be reintegrated in the same cell or transferred via conjugation to a recipient cell.

As used herein, the term “integrating conjugative element” refers to a group of chromosomally integrated, self-transmissible genetic elements.

As used herein, the term “P1-derived artificial chromosome” refers to a DNA construct that originated from the P1 bacteriophage.

As used herein, the term “bacterial artificial chromosome” refers to an engineered DNA sequence used to clone DNA sequences into bacteria.

The term “recombination-mediated genetic engineering genes” or “recombineering genes” refers to genes that assist in creating genetic modifications in a DNA sequence. In instances, recombination-mediated genetic engineering genes allow in vivo construction of constructions in cells (e.g. bacteria cells) without in vitro genetic engineering techniques. In instances, recombination-mediated genetic engineering genes allow for genetic modifications to occur without introduction of enzymes including ligases and restriction enzymes. For example, the genes may be involved in a bacterium's natural process of homologous recombination without traditional molecular biology techniques known in the art. In embodiments, the recombineering genes are lambda red genes. The recombineering genes are Redα, Redβ, and Redγ. For example, the genes may induce homologous recombination at a high rate in bacteria. In a donor cell or recipient cell, expression of one or more recombineering genes may be inducible. In embodiments, the donor cell or the recipient cell includes an oligonucleotide encoding one or more recombination-mediated genetic engineering genes. In embodiments, the oligonucleotide encoding one or more recombination-mediated genetic engineering genes is in the donor cell plasmid. In embodiments, the recombination-mediated genetic engineering genes are inducible. In embodiments, the recombination-mediated genetic engineering genes are Redα, Redβ, and Redγ. In embodiments, the oligonucleotide encoding one or more homologous DNA repair genes is in the recipient cell genome. In embodiments, the oligonucleotide encoding one or more homologous DNA repair genes is in a helper plasmid. In embodiments, the oligonucleotide encoding one or more homologous DNA repair genes is in the recipient plasmid. In embodiments, the oligonucleotide encoding one or more homologous DNA repair genes is in the recipient cell genome.

As used herein, the term “inducible high-copy replication of origin” refers to a plasmid or vector containing a high number of origins of replication, (for example, between 150-200 copies in E. coli plasmid pUC) that are inducible by environmental conditions, such as change in temperature.

As used herein, the term “helper plasmid” is a plasmid that contains genes or other DNA elements necessary for a bacteria to carry out a specified function. A helper plasmid may contain an endonuclease, elements to transfer foreign DNA into the genome, to transfer a plasmid to another cell, or to perform homologous recombination. In embodiments, the helper plasmid is an IncF1 plasmid, an IncPa plasmid, an IncI1 plasmid, a pTiC58 from Agrobacterium tumefaciens, a cAD1 plasmid, an Inc18 plasmid, a pIJ101 from Streptomyces, or an IncH plasmid. In embodiments, the helper plasmid is an IncF1 plasmid. In embodiments, the helper plasmid is an IncPa plasmid. In embodiments, the helper plasmid is an IncI1 plasmid. In embodiments, the helper plasmid is a pTiC58 from Agrobacterium tumefaciens. In embodiments, the helper plasmid is a cAD1 plasmid. In embodiments, the helper plasmid is an IncI8 plasmid. In embodiments, the helper plasmid is a pIJ101 from Streptomyces. In embodiments, the helper plasmid is an IncH plasmid. In embodiments, the helper plasmid lacks a functional origin of transfer. In embodiments, the helper plasmid includes a selectable marker selecting for retention of the helper plasmid in the donor cell.

As used herein, the term “homing endonuclease” refers to an endonuclease that is either encoded as a free-standing gene within an intron sequence, as a fusion with a host protein, or as a self-splicing protein. Homing endonucleases catalyze the hydrolysis of DNA at longer recognition sites, when compared to Group II restriction enzymes. Homing endonuclease examples include, but are not limited to, LAGLIDAG, GIY-YIG, His-Cys box, H-N-H, PD-(D/E)xK, and Vsr-like/EDxHD. In embodiments, the homing endonuclease is I-ScaI, PI-SceI, I-AniI, I-CeuI, I-ChuI, I-CpaI, I-CpaII, I-CreI, I-DmoI, H-DreI, I-HmuI, I-HmuII, I-LlaI, I-MsoI, PI-PfuI, PI-PkoII, I-PorI, I-PpoI, PI-PspI, I-SceI, I-SceII, I-SceIII, I-SceIV, I-SceV, I-SceVI, I-SceVII, I-Ssp6803I, I-TevI, I-TevII, I-TevIII, PI-TliI, PI-TliII, I-Tsp061I, or I-Vdi141I. In embodiments, the homing endonuclease is I-ScaI. In embodiments, the homing endonuclease is PI-SceI. In embodiments, the homing endonuclease is I-AniI. In embodiments, the homing endonuclease is I-CeuI. In embodiments, the homing endonuclease is I-ChuI. In embodiments, the homing endonuclease is I-CpaI. In embodiments, the homing endonuclease is I-CpaII. In embodiments, the homing endonuclease is I-CreI. In embodiments, the homing endonuclease is I-DmoI. In embodiments, the homing endonuclease is H-DreI. In embodiments, the homing endonuclease is I-HmuI. In embodiments, the homing endonuclease is I-HmuII. In embodiments, the homing endonuclease is I-LlaI. In embodiments, the homing endonuclease is I-MsoI. In embodiments, the homing endonuclease is PI-PfuI. In embodiments, the homing endonuclease is PI-PkoII. In embodiments, the homing endonuclease is I-PorI. In embodiments, the homing endonuclease is I-PpoI. In embodiments, the homing endonuclease is PI-PspI. In embodiments, the homing endonuclease is I-SceI. In embodiments, the homing endonuclease is I-SceII. In embodiments, the homing endonuclease is I-SceIII. In embodiments, the homing endonuclease is I-SceIV. In embodiments, the homing endonuclease is I-SceV. In embodiments, the homing endonuclease is I-SceVI. In embodiments, the homing endonuclease is I-SceVII. In embodiments, the homing endonuclease is I-Ssp6803I. In embodiments, the homing endonuclease is I-TevI. In embodiments, the homing endonuclease is I-TevII. In embodiments, the homing endonuclease is I-TevIII. In embodiments, the homing endonuclease is PI-TliI. In embodiments, the homing endonuclease is PI-TliII. In embodiments, the homing endonuclease is I-Tsp061I, or I-Vdi141I. In embodiments, the homing endonuclease is I-Vdi141I.

As used herein, the term “HO” or “Homothallic switching endonuclease” refers to the zinc-finger nuclease in Saccharomyces cerevisiae responsible for initiation of mating type interconversion.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein, “donor cell” refers to a cell (e.g. a bacteria cell) that transfers genetic material to another cell (e.g. a bacteria cell, a plant cell, etc.). The cell that receives the transferred genetic material is referred to herein as a “recipient cell”.

The term “donor plasmid/construct”, as used herein refers to DNA from a donor cell (e.g. a bacteria cell) including an oligonucleotide sequence (e.g. donor DNA, oligonucleotide including a DNA element or fragment thereof) that is to be transferred from the donor cell to a recipient cell (e.g. a bacteria cell, yeast cell, plant cell, etc.). Typically, the donor plasmid is a circular double-stranded DNA that is separate from genomic DNA. Thus, the term “recipient plasmid” refers to plasmid DNA from a recipient cell that receives the donor DNA (e.g. by homologous recombination of the donor DNA into the recipient). The term “recipient oligonucleotide” refers to any oligonucleotide in the recipient cell that receives the donor DNA. In embodiments, the DNA from the donor plasmid is received by DNA other than DNA from a recipient plasmid. Thus, in embodiments, the donor DNA may be incorporated into genomic DNA.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

In embodiments of the methods described here, the donor construct or recipient cell includes an oligonucleotide encoding one or more homologous DNA repair genes. In embodiments, the oligonucleotide encoding the one or more homologous DNA repair genes is in the first, second, or subsequent donor plasmid. In embodiments, homologous DNA repair gene expression is inducible. In embodiments, the homologous DNA repair gene is RecA.

In embodiments of the methods described here, the construct cell or recipient cell includes an oligonucleotide encoding one or more recombination-mediated genetic engineering genes. In embodiments, the oligonucleotide encoding one or more recombination-mediated genetic engineering genes is in the donor cell plasmid.

In embodiments of the methods described here, the recombination-mediated genetic engineering genes are inducible. In embodiments, the recombination-mediated genetic engineering genes are Redα, Redβ, and Redγ. In embodiments, the oligonucleotide encoding one or more homologous DNA repair genes is in the recipient cell genome. In embodiments, the oligonucleotide encoding one or more homologous DNA repair genes is in a helper plasmid. In embodiments, the oligonucleotide encoding one or more homologous DNA repair genes is in the recipient plasmid. In embodiments, the oligonucleotide encoding one or more homologous DNA repair genes is in the recipient cell genome.

In embodiments of the methods described here, donor cells, recipient cells, or recombinant recipient cells may be in an ordered array, or in a first or second ordered array. In embodiments, the donor cells, recipient cells, or recombinant recipient cells may be transferred to positions on a third ordered array, a fourth ordered array, or a subsequent ordered array.

In embodiments of the methods described here, the donor cells and the recipient cells are bacteria cells. In embodiments, the recipient cells are not bacteria cells. In embodiments, the recipient cells are plant cells. In embodiments, the recipient cells are yeast cells. In embodiments, the recipient cells are mammalian cells.

### Methods of Truncating or Assembling DNA Elements

The invention provides methods of truncating and/or assembling a DNA element into a recombined-recipient oligonucleotide within a recipient cell, the method comprising: (a) contacting a first donor cell comprising a first donor plasmid with a recipient cell comprising a recipient oligonucleotide under conditions to (i) transfer the first donor plasmid from the first donor cell to the recipient cell by conjugation and (ii) recombine the first donor plasmid and the recipient oligonucleotide in the recipient cell by homologous recombination, wherein: the first donor plasmid comprises an optional first endonuclease site (C1) and an optional third endonuclease site (C3); and a first oligonucleotide comprising a first homologous recombination region (HR1), a first DNA element (oligo1), a second homologous recombination region (HR2) comprising two homologous recombination regions (HR2.1, HR2.2); optionally wherein HR2.1 or oligo1, and HR2.2 flank a non-homologous region comprising one (C2) or two endonuclease sites (C2.1, C2.2); the recipient oligonucleotide comprises a third homologous recombination region (HR3) homologous to HR1 and a fourth homologous recombination region (HR4) homologous to HR2.2, and optionally a second DNA element (oligo2); thereby providing, following the homologous recombination of HR1 with HR3 and HR2.2 with HR4, a first recombined-recipient oligonucleotide in the recipient cell comprising at least a portion of oligo1 and/or oligo2, wherein HR1 is within oligo1 and/or HR3 is within oligo2, such that the oligo1 and/or oligo2 is truncated. In embodiments, HR3 and HR4 flank a non-homologous region comprising one (C4) or two endonuclease sites (C4.1, C4.2). In embodiments, the recipient oligonucleotide is in a recipient cell plasmid or the recipient cell genome. In embodiments, the first recombined recipient oligonucleotide comprises at least a portion of a gene, a promoter, an enhancer, a terminator, an intron, an intergenic region, a barcode, a guide RNA (gRNA), or a combination thereof. In embodiments, DNA elements oligo1 and/or oligo2 are captured genomic DNA or cDNA.

In other embodiments, the invention method further comprises: (b) contacting a second donor cell comprising a second donor plasmid with the recipient cell comprising the first recombined-recipient oligonucleotide under conditions to (i) transfer the second donor plasmid from the second donor cell to the first recipient cell by conjugation and (ii) recombine the second donor plasmid and the first recombined-recipient oligonucleotide to form a second recombined-recipient oligonucleotide in the recipient cell by homologous recombination, wherein the second donor plasmid comprises an optional fifth endonuclease site (C5) and an optional sixth endonuclease site (C6); and a second oligonucleotide comprising a fifth homologous recombination region (HR5) homologous to HR2.1, optionally a third DNA element (oligo3), optionally a sixth homologous recombination region (HR6) comprising two homologous recombination regions (HR6.1, HR6.2), thereby providing, following the homologous recombination of HR5 with R2.1 and HR6.2 with HR4, a second recombined-recipient oligonucleotide in the recipient cell comprising at least a portion of the oligo1, oligo2 and/or oligo3, wherein HR5 is within oligo3 and/or HR2.1 is within oligo1 or oligo2, such that the oligo1, oligo2, and/or oligo3 is truncated. In embodiments, HR6.1 and HR6.2 flank a non-homologous region comprising one (C7) or two endonuclease sites (C7.1, C7.2). In embodiments, the second recombined-recipient oligonucleotide comprises at least a portion of one or more genes, promoters, enhancers. terminators, introns, intergenic regions, barcodes, guide RNAs (gRNAs), or a combination thereof.

In further embodiments of the invention method, step (b) is repeated for one or more iterations with a third or subsequent donor cell comprising a third or subsequent donor plasmid comprising compatible HR regions and a third or subsequent oligonucleotide comprising a fourth or subsequent DNA element (oligo4, oligo5, . . . oligoN), thereby forming a third or subsequent recombined-recipient oligonucleotide comprising at least a portion of the first, the second, third, fourth and/or subsequent DNA elements which together form a third or subsequent recombined-recipient oligonucleotide. In embodiments, step (a) comprises a plurality of first donor cells, each comprising a different first donor plasmid; and step (b) comprises a plurality of second, third, or subsequent donor cells, each comprising a different second, third, or subsequent donor plasmid; optionally wherein each first donor cell is in a position in a first ordered array and each second, third, or subsequent donor cell is in a position in a second, third, or subsequent ordered array; optionally wherein the method generates a combinatorial library comprising a plurality of different assembled DNA elements. In embodiments, the combinatorial library selected from the group consisting of: a yeast artificial chromosome (YAC), a bacterial artificial chromosome (BAC), a mammalian artificial chromosome (MAC), a human artificial chromosome (HAC), and/or a plant artificial chromosome. In embodiments, the donor plasmid or recipient oligonucleotide is a viral vector. In embodiments, the donor plasmid comprises an oligonucleotide that enables plasmid conjugation. In embodiments, the donor plasmid or the recipient cell comprises an oligonucleotide encoding one or more homologous DNA repair genes; optionally wherein expression of the one or more homologous DNA repair genes is inducible. In embodiments, the donor plasmid or recipient cell comprises an oligonucleotide encoding one or more recombination-mediated genetic engineering genes. In embodiments, the donor cell and the recipient cell are independently a bacteria cell; optionally wherein the bacteria cell is E. coli, Vibrio natriegens or V. cholerae. In embodiments, the assembled DNA element is from 100 nucleotides to 500,000 nucleotides in length. In embodiments, the first, second or subsequent homologous recombination (HR) regions and their corresponding HR regions on the recipient oligonucleotide each comprise from about 20 base pairs to about 500 base pairs; optionally about 50 to 100 base pairs. In embodiments, the bridging oligonucleotides has a length in a range selected from the group consisting of: from 30-900, from 40-800, from 50-700, from 50-600, from 50-500, from 50-400, from 50-300, from 50-200, from 60-150, from 70-125, and/or from 80-100 nucleotides in length. In embodiments, the number of base pairs that are truncated from the recipient oligonucleotide is selected from the group consisting of: 50 bp-200 Kb, 100 bp-150 Kb, 200 bp-125 Kb, 300 bp-100 Kb, 400 bp-90 Kb, 500 bp-80 Kb, 600 bp-70 Kb, 700 bp-60 Kb, 800 bp-50 Kb, 900 bp-40 Kb, 1000 bp-30 Kb.

In embodiments, the method further comprises one or more steps of lysing the recipient cells; amplifying an assembled DNA element; isolating an assembled DNA element; isolating a recipient oligonucleotide; sequencing an assembled DNA element; and sequencing a recipient oligonucleotide. In embodiments, the steps of contacting the first and second or subsequent donor cells with the first recipient cell are performed simultaneously; optionally wherein only a final donor plasmid comprises a selectable marker or each donor plasmid comprises a selectable marker not present on the recipient oligonucleotide.

In embodiments, the donor plasmid comprising the last DNA element to form part of an assembled DNA element comprises a barcode homologous recombination (BHR) region to produce recipient cells each containing a recombined recipient oligonucleotide comprising the assembled DNA element the BHR, and a further HR; and the method further comprises (i) constructing or acquiring an array of barcode donor cells, each containing a barcode donor plasmid comprising an HR homologous to the BHR, a unique barcode oligonucleotide, and a second HR homologous to the further HR of the recombined recipient oligonucleotide; (ii) contacting the array of barcode donor cells with an array of the recipient cells under conditions to (a) transfer the barcode donor plasmids from the barcode donor cells to the recipient cells by conjugation and (b) recombine the barcode donor plasmids and recipient oligonucleotides in the recipient cells by homologous recombination, thereby producing an array of recipient cells comprising barcoded assemblies.

In embodiments, each donor plasmid comprises a further pair of unique endonuclease sites CX, CY, flanking a barcode homologous recombination (BHR) region and the method further comprises contacting an array of recipient cells, each comprising a DNA assembly, with an array of barcode donor cells, each containing a barcode donor plasmid comprising a pair of HR regions homologous to the BHR flanking a unique barcode oligonucleotide, to produce an array of recipient cells comprising barcoded assemblies.

In embodiments, the DNA assembly methods may further comprise contacting a reset donor cell comprising a reset donor plasmid with a recipient cell comprising a recombined recipient oligonucleotide, wherein the reset donor plasmid comprises, in sequential order, a homologous recombination region (HRt) homologous to a terminal sequence of the DNA assembly, a reset endonuclease site, a selectable marker, a reset endonuclease site, a homologous recombination region (HRX), and an origin of transfer, wherein the recombined recipient oligonucleotide comprises, in sequential order, a reset endonuclease site, the DNA assembly, a homologous recombination region homologous to HRX (HRXa) and a reset endonuclease site, thereby providing, subsequent to homologous recombination between the HRt and the terminal sequence of the DNA assembly and between the HRX and the HRXa, a reset plasmid comprising the origin of transfer and the DNA assembly. In embodiments, the reset plasmid is in a donor cell. In embodiments, the reset plasmid contains a restricted origin of replication that functions in both donor cells and recipient cells. In embodiments, the reset donor plasmid is constructed by a method comprising introducing an oligonucleotide insert comprising homologous recombination regions HRt, HRX, flanking two endonuclease sites (C1, C2) and a counter-selectable marker (CM), HRt-C1-CM-C2-HRX; or a library of such oligonucleotide inserts; allowing an endonuclease to cleave the endonuclease sites and introducing a counter-selectable marker at the cleavage sites using homologous recombination.

The invention also provides methods of conjugating barcodes to oligonucleotides, the method comprising (a) inserting each oligonucleotide of a mixture of oligonucleotides into a donor plasmid, each donor plasmid comprising, in sequential order, optionally a first endonuclease site (C1), a first homologous recombination region (HR1), a second homologous recombination region (HR2), and optionally a second endonuclease site (C2); wherein each oligonucleotide is inserted between HR1 and HR2, thereby providing a plurality of donor plasmids comprising donor oligonucleotides, each donor plasmid comprising a single donor oligonucleotide from the mixture of oligonucleotides: C1-HR1-oligo-HR2-C2; (b) transforming a plurality of cells with the plurality of donor plasmids such that each cell comprises a donor plasmid, thereby forming a plurality of donor cells; (c) plating and culturing the plurality of donor cells, each in a unique position on a first ordered array, thereby providing a first ordered array of donor cells; (d) providing a plurality of recipient cells in a second ordered array, wherein each recipient cell comprises a recipient oligonucleotide comprising, in sequential order, a unique barcode sequence, wherein the unique barcode sequence identifies a position of the recipient cell in the second ordered array, a third homologous recombination region (HR3) homologous to HR1, optionally a third endonuclease site (C3), and a fourth homologous recombination region (HR4) homologous to HR2; (e) contacting the first ordered array of donor cells with the second ordered array of recipient cells under conditions to (i) transfer the donor plasmids from the donor cells to the recipient cells in corresponding positions on the array by conjugation, (ii) optionally cleave the first, second, and third endonuclease sites, and (ii) transfer the oligonucleotides from the donor plasmids to the recipient cell oligonucleotides by homologous recombination, thereby forming an third array of fusion oligonucleotides, each comprising a unique barcode sequence and a donor oligonucleotide from the mixture of oligonucleotides; and (f) optionally sequencing the fusion oligonucleotides, and thereby identifying each oligonucleotide in the array of by its barcode sequence. In embodiments, the recipient oligonucleotide is in a recipient cell plasmid or the recipient cell genome. In embodiments, the donor plasmid comprises a selectable marker between HR1 and HR2 selecting for integration of the oligonucleotide into the recipient cell oligonucleotide; optionally wherein the donor plasmid comprises a counter-selectable marker. In embodiments, the recipient cell oligonucleotide comprises a fourth endonuclease site (C4).

In a further aspect is provided a method of assembling a DNA element. The method includes: (a) providing a first host cell including a first donor plasmid including, in sequential order: (i) a first endonuclease target site, (ii) a first homologous recombination region, (iii) optionally a first oligonucleotide including a first DNA element, (iv) a second homologous recombination region, (v) a second endonuclease target site, (vi) and a third endonuclease target site, (b) providing a recipient cell, wherein the recipient cell includes a recipient oligonucleotide including: (i) a third homologous recombination region, wherein the third homologous region is homologous to the first homologous recombination region, (ii) a fourth endonuclease target site, and (iii) a fourth homologous region, wherein the fourth homologous recombination region is homologous to the second homologous recombination region; and (c) contacting the first host cell with the recipient cell under conditions to (i) transfer the first donor plasmid from the first host cell to the recipient cell by bacterial conjugation, (ii) direct a first endonuclease to the at least one of the first endonuclease target site, the third endonuclease target site, or the fourth endonuclease target site, thereby producing double-stranded breaks in the first donor plasmid and the recipient oligonucleotide and (iii) recombine the first donor plasmid and the recipient oligonucleotide in the recipient cell by homologous recombination via the first and second homologous recombination regions with the third and fourth corresponding homologous recombination regions, thereby forming a recombined recipient oligonucleotide. The method may further include: (d) providing a second host cell including a second donor plasmid including, in sequential order: (i) a fifth endonuclease target site, (ii) a fifth homologous recombination region which is homologous to the second homologous region, (iii) a second oligonucleotide including a second DNA element, (iv) a sixth homologous recombination region which is homologous to the fourth homologous region, and (v) a sixth endonuclease target site; (e) contacting the second host cell with the recipient cell containing the recombined recipient oligonucleotide under conditions to (i) transfer the second donor plasmid from the second host cell to the recipient cell by bacterial conjugation, (ii) express a second endonuclease, (iii) direct the second endonuclease to the second endonuclease target site, the fifth endonuclease target site and/or the sixth endonuclease target site, thereby producing double-stranded breaks, (iv) recombine the second donor plasmid and the recombined recipient oligonucleotide in the recipient cell by homologous recombination via the fifth and sixth homologous recombination sites with the corresponding second and fourth homologous recombination sites, thereby forming a second recombined recipient oligonucleotide including an assembled DNA element. In some embodiments, a different portion of the fourth homologous region is homologous to the sixth homologous recombination region, compared to the portion of the fourth homologous region that is homologous to the second homologous recombination region.

In embodiments, step (a) includes a plurality of first host cells, wherein each cell includes a unique first oligonucleotide. In embodiments, step (d) includes a plurality of second host cells, wherein each cell includes a unique second oligonucleotide. In embodiments, each first host cell includes a unique plasmid. In embodiments, each second host cells includes a unique plasmid. In embodiments, each first host cell is in a position in a first ordered array. In embodiments, a plurality of first host cells are in a position in a first ordered array, thereby forming a pool of first host cells in the first ordered array. In embodiments, each second host cell is in a position in a second ordered array. In embodiments, a plurality of second host cells are in a position in a second ordered array, thereby forming a pool of second host cells in each position in the second ordered array.

In embodiments, the first donor cell is in a first ordered array, the second donor cell is in a second ordered array, and one or more subsequent donor cells are in one or more subsequent arrays. Thus, in embodiments, the method provided herein generates a variant library including a plurality of different assembled DNA elements. In embodiments, the variant library is generated by 1) making each variant independently using a first host cell, a second host cell, or a subsequent host cell in a position in a first, second array, or subsequent array or 2) generating a variant pool using a plurality of first host cells, second host cells, or subsequent host cells in a position in a first, second array, or subsequent array. In embodiments, the variant library is generated by making each variant independently using a first host cell, a second host cell, or a subsequent host cell in a position in a first, second array, or subsequent array. In embodiments, the variant library is generated by generating a variant pool using a plurality of first host cells, second host cells, or subsequent host cells in a position in a first, second array, or subsequent array. For example, for the method provided herein including embodiments thereof, the first DNA element and/or second DNA element may be a DNA barcode or plurality of DNA barcodes. In embodiments, the method generates a recursive barcoding platform. For example, in embodiments wherein the first DNA element and/or second DNA element is a DNA barcode or plurality of DNA barcodes, the method can be used for tracking of cell lineages.

For example, the first DNA element and/or DNA element may be a gRNA or a plurality of gRNAs. Thus, in embodiments, the method includes generation of combinatorial gRNA libraries.

In embodiments, the first endonuclease targets the first endonuclease target site. In embodiments, the first endonuclease targets the third endonuclease target site. In embodiments, the first endonuclease targets the fourth endonuclease target site. In embodiments, the second endonuclease targets the second endonuclease target site. In embodiments, the second endonuclease targets the fifth endonuclease target site. In embodiments, the second endonuclease targets the sixth endonuclease target site.

In embodiments, the DNA element is a gene. In embodiments, the DNA element is a promoter. In embodiments, the DNA element is an enhancer. In embodiments, the DNA element is a terminator. In embodiments, the DNA element is an intron. In embodiments, the DNA element is an intergenic region. In embodiments, the DNA element is a barcode. In embodiments, the DNA element is a translation initiation site. In embodiments, the DNA element is a gRNA. In embodiments, the DNA element is a fragment of any of the foregoing.

In embodiments, the recipient oligonucleotide is in a recipient plasmid. In embodiments, the recipient oligonucleotide is in the recipient cell genome.

In embodiments, the second donor plasmid further includes a seventh homologous recombination region and a seventh endonuclease target site between the components of (d) iii) and (d) iv). In embodiments, the first endonuclease targets the seventh endonuclease site.

In embodiments, the donor plasmid includes an oligonucleotide encoding a gRNA, and the recipient cell includes an oligonucleotide encoding an RNA-guided DNA endonuclease. In embodiments, the donor plasmid includes an oligonucleotide encoding a gRNA, and wherein the recipient cell genome includes an oligonucleotide encoding an RNA-guided DNA endonuclease. In embodiments, the donor plasmid includes an oligonucleotide encoding a gRNA, and the recipient plasmid includes an oligonucleotide encoding an RNA-guided DNA endonuclease. In embodiments, the donor plasmid includes an oligonucleotide encoding a gRNA, and a recipient helper plasmid includes an oligonucleotide encoding an RNA-guided DNA endonuclease. In embodiments, the donor plasmid includes an oligonucleotide encoding a gRNA, and the donor plasmid includes an oligonucleotide encoding an inducible RNA-guided DNA endonuclease. In embodiments, the donor plasmid includes an oligonucleotide encoding a gRNA, and the recipient genome includes an oligonucleotide encoding an inducible RNA-guided DNA endonuclease. In embodiments, the donor plasmid includes an oligonucleotide encoding a gRNA, and the recipient plasmid includes an oligonucleotide encoding an inducible RNA-guided DNA endonuclease. In embodiments, the donor plasmid includes an oligonucleotide encoding a gRNA, and a recipient helper plasmid includes an oligonucleotide encoding an inducible RNA-guided DNA endonuclease.

In embodiments, the recipient cell includes an inducible gRNA. In embodiments, the donor cell includes an oligonucleotide encoding an RNA-guided DNA endonuclease. In embodiments, the recipient cell includes an oligonucleotide encoding an RNA-guided DNA endonuclease. In embodiments, expression of the RNA-guided DNA is constitutive. In embodiments, expression of the RNA-guided DNA is inducible.

In embodiments, the RNA-guided DNA endonuclease is Cas9. In embodiments, the RNA-guided DNA endonuclease is Cas10. In embodiments, the RNA-guided DNA endonuclease is Cpf1. In embodiments, the RNA-guided DNA endonuclease is C2c1. In embodiments, the RNA-guided DNA endonuclease is C2c2. In embodiments, the RNA-guided DNA endonuclease is C2c3. In embodiments, the RNA-guided DNA endonuclease is Cas12c1. In embodiments, the RNA-guided DNA endonuclease is Cas12a. In embodiments, the RNA-guided DNA endonuclease is Cas12b. In embodiments, the RNA-guided DNA endonuclease is Cas12c2. In embodiments, the RNA-guided DNA endonuclease is Cas12g. In embodiments, the RNA-guided DNA endonuclease is Cas12e. In embodiments, the RNA-guided DNA endonuclease is Cas12i1. In embodiments, the RNA-guided DNA endonuclease is Cas12i2.

For the methods provided herein, in embodiments, an oligonucleotide encoding the first endonuclease is in the donor plasmid. In embodiments, an oligonucleotide encoding the first endonuclease is the recipient oligonucleotide. In embodiments, an oligonucleotide encoding the first endonuclease is in a recipient cell helper plasmid. In embodiments, an oligonucleotide encoding the first endonuclease is in the recipient genome. In embodiments, expression of the first endonuclease is inducible. In embodiments, the oligonucleotide encoding the second endonuclease is in the donor plasmid. In embodiments, an oligonucleotide encoding the second endonuclease is in the recipient oligonucleotide. In embodiments, an oligonucleotide encoding the second endonuclease is in a recipient cell helper plasmid.

In embodiments, the first endonuclease and/or the second endonuclease is a homing endonuclease. In embodiments, the homing endonuclease is I-ScaI, PI-SceI, I-AniI, I-CeuI, I-ChuI, I-CpaI, I-CpaII, I-CreI, I-DmoI, H-DreI, I-HmuI, I-HmuII, I-LlaI, I-MsoI, PI-PfuI, PI-PkoII, I-PorI, I-PpoI, PI-PspI, I-SceI, I-SceII, I-SceIII, I-SceIV, I-SceV, I-SceVI, I-SceVII, I-Ssp6803I, I-TevI, I-TevII, I-TevIII, PI-TliI, PI-TliII, I-Tsp061I, or I-Vdi141I. In embodiments, the homing endonuclease is I-ScaI. In embodiments, the homing endonuclease is PI-SceI. In embodiments, the homing endonuclease is I-AniI. In embodiments, the homing endonuclease is I-CeuI. In embodiments, the homing endonuclease is I-ChuI. In embodiments, the homing endonuclease is I-CpaI. In embodiments, the homing endonuclease is I-CpaII. In embodiments, the homing endonuclease is I-CreI. In embodiments, the homing endonuclease is I-DmoI. In embodiments, the homing endonuclease is H-DreI. In embodiments, the homing endonuclease is I-HmuI. In embodiments, the homing endonuclease is I-HmuII. In embodiments, the homing endonuclease is I-LlaI. In embodiments, the homing endonuclease is I-MsoI. In embodiments, the homing endonuclease is PI-PfuI. In embodiments, the homing endonuclease is PI-PkoII. In embodiments, the homing endonuclease is I-PorI. In embodiments, the homing endonuclease is I-PpoI. In embodiments, the homing endonuclease is PI-PspI. In embodiments, the homing endonuclease is I-SceI. In embodiments, the homing endonuclease is I-SceII. In embodiments, the homing endonuclease is I-SceIII. In embodiments, the homing endonuclease is I-SceIV. In embodiments, the homing endonuclease is I-SceV. In embodiments, the homing endonuclease is I-SceVI. In embodiments, the homing endonuclease is I-SceVII. In embodiments, the homing endonuclease is I-Ssp6803I. In embodiments, the homing endonuclease is I-TevI. In embodiments, the homing endonuclease is I-TevII. In embodiments, the homing endonuclease is I-TevIII. In embodiments, the homing endonuclease is PI-TliI. In embodiments, the homing endonuclease is PI-TliII. In embodiments, the homing endonuclease is I-Tsp061I, or I-Vdi141I. In embodiments, the homing endonuclease is I-Vdi141I.

In embodiments, the endonuclease is a transcription activator-like effector nuclease. In embodiments, the endonuclease is a zinc finger nuclease.

For the methods provided herein, in embodiments, steps (d) to (e) are repeated for one or more iterations, thereby forming one or more subsequent assembled DNA elements. In embodiments, the first, second or subsequent donor plasmid includes a selectable marker selecting for integration of the first oligonucleotide, second oligonucleotide or subsequent oligonucleotide into the recipient cell oligonucleotide. In embodiments, the first donor plasmid includes a selectable marker selecting for integration of the first oligonucleotide into the recipient cell oligonucleotide. In embodiments, the second donor plasmid includes a selectable marker selecting for integration of the second oligonucleotide into the recipient cell oligonucleotide. In embodiments, the subsequent donor plasmid includes a selectable marker selecting for integration of the subsequent oligonucleotide into the recipient cell oligonucleotide

In embodiments, the first donor plasmid includes a selectable marker selecting for integration of the first oligonucleotide into the recipient oligonucleotide. In embodiments, the selectable marker is between the components of (a)(v) and (a)(iv). In embodiments, the second donor plasmid includes a selectable marker selecting for integration of the second oligonucleotide into the recipient oligonucleotide.

In embodiments, the recipient cell oligonucleotide includes a counter-selectable marker selecting for integration of the first oligonucleotide into the recipient cell oligonucleotide. In embodiments, the recombined recipient cell oligonucleotide includes a counter-selectable marker selecting for integration of the second or subsequent oligonucleotide into the recombined recipient cell oligonucleotide. Counter-selectable markers for use in the methods described herein are described above.

In embodiments, the assembled DNA element, which may also be referred to as a DNA assembly, is sequenced. In embodiments, the recipient oligonucleotide is sequenced. In embodiments, the recombinant recipient oligonucleotide is sequenced. In embodiments, the recombinant recipient oligonucleotide is a plasmid, wherein the plasmid is linearized, ligated to sequencing adaptors and sequenced. In embodiments, the assembled DNA element is amplified by PCR and sequenced. In embodiments, (a) the recipient cells are lysed, (b) the oligonucleotides are digested with an endonuclease or a plurality of endonucleases, (c) the assembled DNA element is isolated, and (d) the assembled DNA element or plurality of assembled genes are ligated to sequencing adaptors and sequenced. In embodiments, the assembled DNA element is isolated. In embodiments, the recombinant recipient oligonucleotide is isolated.

In embodiments, the assembled DNA element is from about 100 nucleotides to about 500,000 nucleotides in length. The length may be any value or subrange within the indicated ranges, including endpoints.

In embodiments, the assembled DNA element is from about 100 nucleotides, about 1000 nucleotides, about 10,000 nucleotides, about 20,000 nucleotides, about 40,000 nucleotides, about 60,000 nucleotides, about 80,000 nucleotides, about 100,000 nucleotides, about 120,000 nucleotides, about 140,000 nucleotides, about 160,000 nucleotides, about 180,000 nucleotides, about 20,000 nucleotides, about 240,000 nucleotides, about 260,000 nucleotides, about 280,000 nucleotides, about 300,000 nucleotides, about 320,000 nucleotides, about 340,000 nucleotides, about 360,000 nucleotides, about 380,000 nucleotides, about 400,000 nucleotides, about 420,000 nucleotides, about 440,000 nucleotides, about 460,000 nucleotides, about 480,000 nucleotides, or about 500,000 nucleotides in length. The length may be any value or subrange within the indicated ranges, including endpoints.

In embodiments, the first, second or subsequent homology regions and the corresponding first, second or subsequent homology regions are about 20 base pairs to about 500 base pairs in length. The length may be any value or subrange within the indicated ranges, including endpoints.

In embodiments, the first, second or subsequent homology regions and the corresponding first, second or subsequent homology regions are about 20 base pairs, 40 base pairs, 60 base pairs, 80 base pairs, 100 base pairs, 120 base pairs, 140 base pairs, 160 base pairs, 180 base pairs, 200 base pairs, 220 base pairs, 240 base pairs, 260 base pairs, 280 base pairs, 300 base pairs, 320 base pairs, 340 base pairs, 360 base pairs, 380 base pairs, 400 base pairs, 420 base pairs, 440 base pairs, 460 base pairs, 480 base pairs or 500 base pairs in length. In embodiments, the first, second or subsequent homology regions and the corresponding the first, second or subsequent homology regions are about 50 base pairs in length. The length may be any value or subrange within the indicated ranges, including endpoints.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Methods for In Vivo DNA Stitching

Example 1 is shown in U.S. Prov. Pat. App. No. 63/682,315, filed Aug. 12, 2024, which is incorporated by reference herein in its entirety as well as Int. Pat. Pub. WO2022187697A1, which is incorporated by reference herein in its entirety.

### Example 2: Bridging Existing DNA Blocks

As depicted in FIG. 7, bridging-oligonucleotide sequences that are 80-100 bp long were used to stitch together DNA blocks without homology. To stitch together parts of existing DNA blocks, the first 40-50 bp of the bridging-oligonucleotide sequence was designed to match the last 40-50 bp of the desired region in the preceding DNA block, and the second 40-50 bp was designed to match the first 40-50 bp of the desired region in the next incoming DNA block.

To construct the donor plasmids comprising the bridging-oligonucleotides, a set of bridging-oligonucleotide sequences, flanked by AscI and NotI restriction sites, was designed on a DNA element and ordered from TWIST. The TWIST-obtained DNA element was then digested using AscI and NotI restriction enzymes, and fragments of the expected size were gel extracted. The extracted gel fragment was ligated with AscI and NotI digested odd or even donor plasmids using T4 ligase and transformed into the dSL2 donor strain. Cells carrying the correct bridging-oligonucleotide donor plasmid were selected on LB+100 μg/ml Hyg+2 μg/ml Tc agar plates for odd donor plasmids, and with LB+60 μg/ml Sp+2 μg/ml Tc agar plates for even donor plasmids.

For stitching together two DNA blocks where one is in an odd donor plasmid or the other is in an even donor plasmid, only one stitch with a donor plasmid containing the bridging-oligonucleotide sequence is necessary (e.g., odd—even bridge—odd). However, for stitching together pairs of DNA blocks where both are in odd donor plasmids or even donor plasmids, two stitching steps are required to ensure that the selection and counter-selection markers are in the correct orientation (e.g., odd—even bridge 1—odd bridge 2—even). In these cases, the bridging-oligonucleotide sequence is the same for both odd and even bridge donor plasmids. Because the bridging-oligonucleotide sequence remains the same, the assembled product on the recipient remains the same; only the downstream selection and counter-selection cassettes are exchanged.

As depicted in FIG. 7, bridging oligonucleotide B10 was used to truncate a DNA element originally used in the assembly of Malbrachea aurantiaca malbrancheamide biosynthetic gene cluster and join it with a DNA element originally used in a synthetic DNA construct (Synthetic DNA 2). Next, B11 was used to truncate the recombinant construct at a site in Synthetic DNA 2 and add part of a DNA element originally used in a Synthetic DNA 3, truncating off the front of the Synthetic DNA 3 fragment. Next, B12 was used to seamlessly join the resulting recombinant construct with another DNA element originally used in the assembly of Malbrachea aurantiaca malbrancheamide. This procedure was performed at 12-fold replication and the resulting constructs were sequence verified. The purity score indicates the fraction of sequence-perfect constructs for each replicate.

### Example 3: High-Throughput DNA Engineering by Mating Bacteria

We introduce SCRIVENER (Sequential Conjugation and Recombination for In Vivo Elongation of Nucleotides with low ERrors), an in vivo DNA assembly platform that streamlines and scales DNA engineering. SCRIVENER combines bacterial conjugation, in vivo DNA cutting, and homologous recombination to stitch DNA blocks together by mating E. coli in large arrays or pools. This approach is simpler, cheaper, and higher throughput than methods requiring DNA to be moved in and out of cells. We perform over 5,000 assemblies with two to 19 blocks (240 bp-12 kb) and assemble constructs up to 81 kb with high fidelity. Most errors are deletions between long repeats, but SCRIVENER minimizes their impact by enabling high-replication assembly and sequence verification at a nominal additional cost per replicate. The platform enables combinatorial library construction and DNA block reuse without PCR, making it a powerful tool to accelerate DNA design-build-test-learn cycles.

Scalable information processing platforms, such as those that handle written language or computer code, have sparked technology innovation cycles that develop applications to sit on top of these base layers. DNA holds promise to become the next major information medium, with emerging applications that use long DNA constructs to produce small molecules, multispecific antibodies, gene therapies, cellular therapies, and organisms designed to purpose.

However, DNA engineering is currently cumbersome, creating a bottleneck to the development of some DNA-based applications. For example, workflows for DNA assembly, such as restriction enzyme cloning, Gibson assembly¹, and Golden Gate assembly^{2,3}, can be idiosyncratic, expensive, and difficult to automate and scale. Clonal DNA must be purified from cells, manipulated using biochemistry and/or molecular biology approaches, and placed back into cells for cloning, amplification, and sequence verification. Scaling these in vivo→in vitro→in vivo DNA assembly workflows often requires construction of large centralized biofoundries with specialized equipment and experienced production teams.^{4,5}

Thus, improving accessibility and reducing the operational friction of large scale DNA engineering has the potential to decentralize, reduce cost, and increase the pace of development of some DNA-based applications. To address this challenge, we introduce SCRIVENER, which combines elements of the MAGIC subcloning system⁶ with elements of the GENESIS and BASIS E. coli-based genome-scale assembly systems^{7,8} to enable assembly and sequence verification of long and difficult DNA constructs on plasmids at high throughput. SCRIVENER offers distinct advantages over existing DNA assembly and verification systems. Once DNA fragments are cloned into bacteria, the entire assembly process is as simple as mating and growing bacteria in 96- or 384-position formats. Arrays of uniquely barcoded recipient cell plasmids allow colonies to be pooled before DNA isolation and sequencing, significantly reducing the cost and time required for whole-plasmid sequence verification. Additionally, SCRIVENER streamlines the reuse of DNA fragments and assembled products, facilitating modular DNA assembly without PCR or other idiosyncratic in vitro manipulations.

### Results

### Overview of SCRIVENER

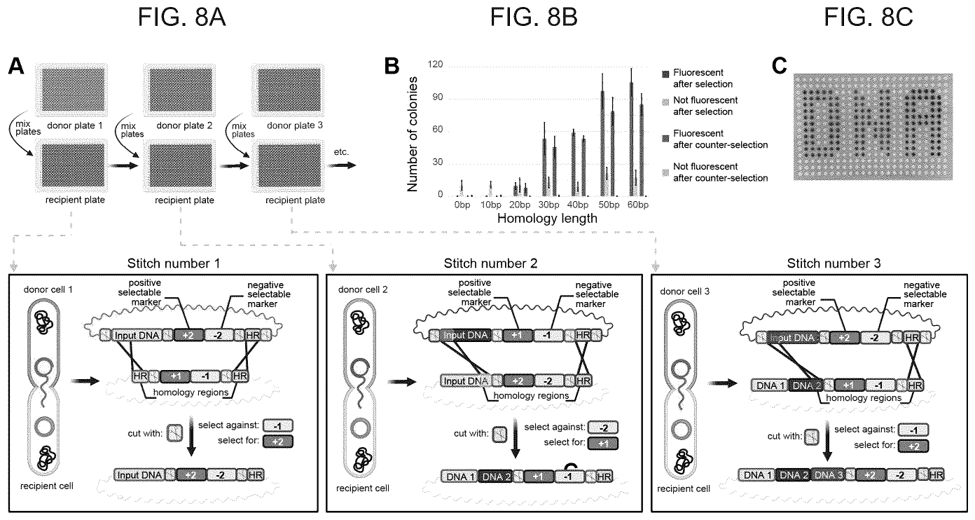

SCRIVENER sequentially and seamlessly assembles DNA blocks on plasmids simply by mating bacteria (FIG. 8A). DNA blocks are inserted into a fixed location on a ‘swapping cassette’ in donor plasmids and transformed into donor cells using standardized methods. Donor plasmids contain an origin of transfer (OriT) and are transferred via F-plasmid mediated conjugation from donor cells to recipient cells, which contain a recipient plasmid with a growing assembly next to another swapping cassette. A genetic program in the recipient cell cuts the swapping cassettes from both the donor and recipient plasmids using CRISPR/Cas9^{9,10} and “stitches” the incoming cassette into the recipient plasmid using the lambda Red homologous recombinase to extend an assembly¹¹. Homology regions designed at the end of the incoming DNA blocks ensure that assemblies are seamless. Swapping cassettes feature alternating selectable and counter-selectable markers, facilitating iterative stitching. As in the MAGIC system and in contrast to BASIS and GENESIS, donor plasmids have a restricted origin of replication that is non-functional in recipient cells, ensuring they are lost during cell outgrowth and minimizing the possibility of unwanted recombination events. Recipient plasmid backbones contain a selectable marker (not shown in FIG. 8A), allowing for the selection of recipient cells and the elimination of donor cells. These design features ensure that most cells passing through the selection and counter-selection steps contain the desired product. DNA constructs are assembled on ColE1 plasmids, making it easy to extract sufficient DNA yield for downstream applications.

### DNA Stitching Fidelity, Efficiency, and Homology Requirements

We first tested SCRIVENER by assembling the fluorophore mPapaya¹² by sequentially stitching four DNA blocks (244 bases each) that contained 50-65 bp of overlapping homology. We successfully assembled the full mPapaya gene in liquid media and on agar, with all four independent replicates in both conditions being fluorescent and sequence perfect after the fourth stitch. To test how stitching accuracy and efficiency depends on the length of homology, we performed the final fluorescence-conferring stitch with DNA blocks that contained between 0 bp and 60 bp of homology (FIG. 8B). Fluorescent colonies were recoverable with as little as 20 bp of homology, but shorter homology lengths resulted in fewer colonies (reduced stitching efficiency) and a higher ratio of non-fluorescent colonies after selection (reduced stitching fidelity). After counter-selection, only fluorescent colonies remained in all conditions, indicating that counter-selection effectively removes incorrect recipient plasmids. Using this assay and cell counts over the course of the procedure, we estimated a stitching efficiency of ˜10⁻⁵ in liquid for stitching blocks with 50 bp of homology. For experiments that follow, we typically chose homology regions with lengths of 45-50 bp, while avoiding sequence features that may lower homologous recombination efficiency and fidelity, such as repetitive sequences, secondary structure, homopolymers, and extreme GC content.

### DNA Stitching and Sequence Verification in Bacterial Arrays

We next developed mid- and high-throughput protocols to multiplex SCRIVENER. For the mid-throughput protocol, we limited our toolset to those found in a standard molecular biology lab (96-well plates and multi-channel pipettes) with the aim of enabling most labs to perform hundreds of assemblies in parallel. For the high-throughput protocol, we assembled on 96- or 384-position arrays on agar with off-the-shelf pinning robots. We first tested both methods in a 96-position format with an expanded set of three fluorophores (mPapaya, mPlum, sfGFP, 4 stitches each) at 32-fold replication each. To test sequence fidelity in high throughput, we created arrays of recipient cells with unique DNA barcodes at each position. Assemblies on these pre-barcoded plasmids allowed us to pool arrays prior to DNA isolation and Oxford Nanopore Technologies (ONT) sequencing, significantly reducing the cost and time for whole-plasmid sequence verification.¹³ Using this sequencing method and a custom analysis pipeline, we found that 94 of 95 and 69 of 73 positions with sufficient sequencing coverage contained no errors using the liquid-based and agar-based protocols, respectively (STAR Methods). We then tested a four-stitch assembly of the mPapaya and lacZ reporters in a 384-position format on agar. A functional reporter was observed at every position (FIG. 8C). These results demonstrate that many independent replicates of a construct can be assembled and sequence-verified at a nominal additional cost and time per replicate (Table 1).

SCRIVENER Performance with Long and Challenging DNA Sequences

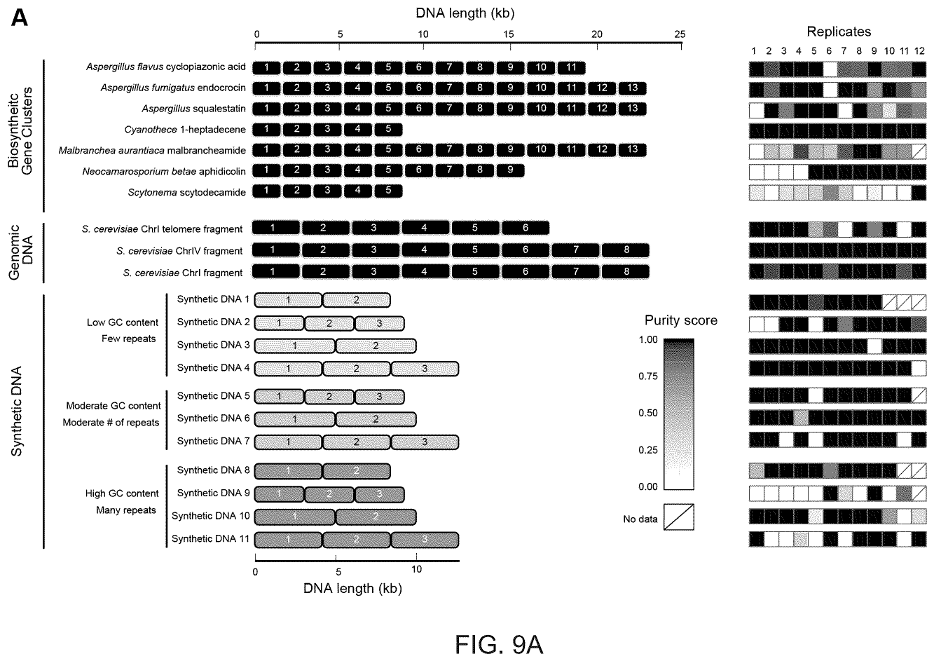

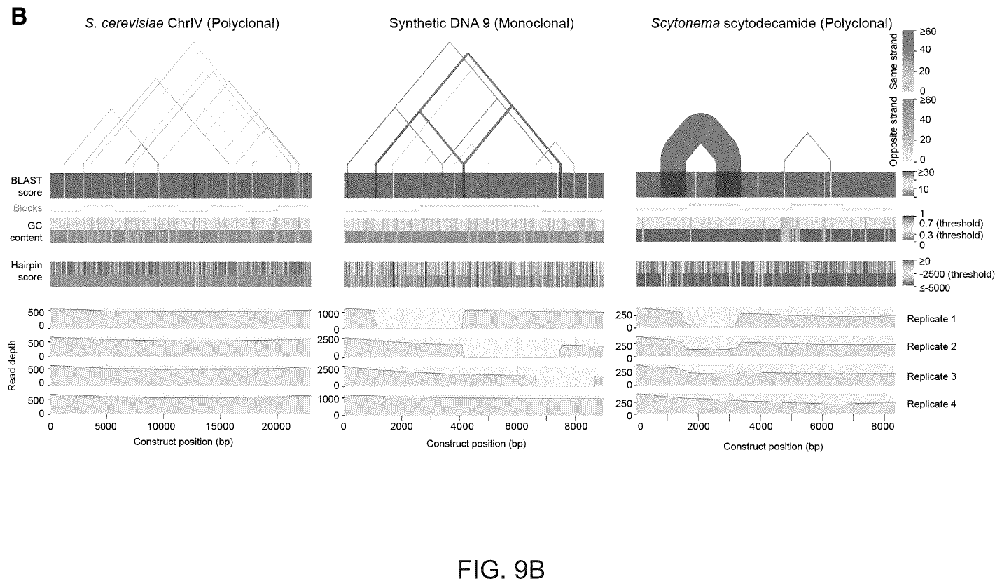

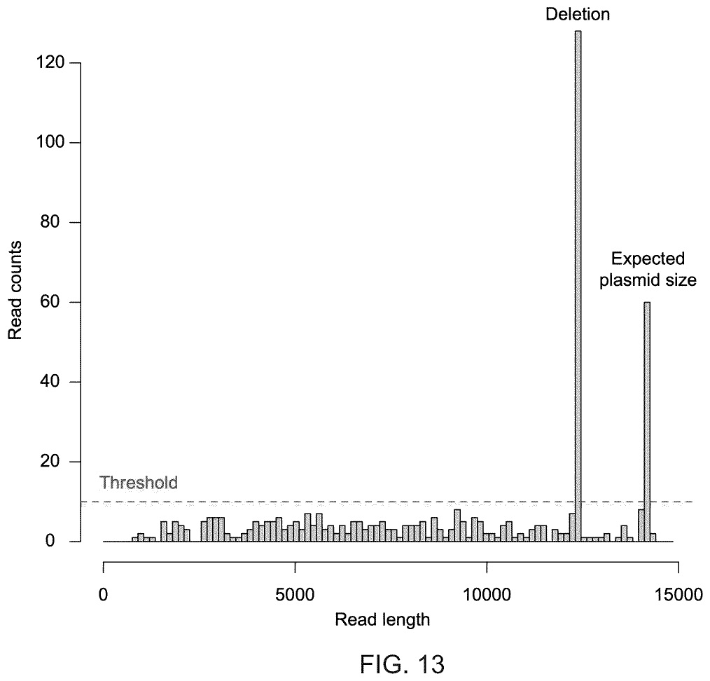

Sequence features such as extreme GC content, homopolymers, tandem repeats, DNA secondary structure, long interspersed repeats, and long overall construct lengths¹⁴ are challenging to assemble by in vitro assembly methods that include PCR or DNA annealing steps. To test SCRIVENER performance across some of these features, we designed a test set of 21 constructs that includes natural Biosynthetic Gene Clusters (BGCs), regions of the yeast Saccharomyces cerevisiae genome (including a telomeric region and several regions that major DNA synthesis providers could not synthesize,), and synthetic DNA constructs that contain different levels of overall GC content, DNA secondary structure, and interspersed repeats (FIG. 9). These assemblies used building blocks ranging from 1.8 kb to 5 kb to construct 8 kb to 23 kb constructs using 2 to 13 stitches at 12-fold replication each (FIG. 9B). To determine the assembly fidelity, we sequenced each assembly replicate either before (BGC and yeast genome assemblies) or after (synthetic DNA assemblies) isolation of a single cell at each position. Prior to single cell isolation, we expect each colony to be polyclonal, containing multiple competing cell lineages that stem from independent stitching events. A fraction of these lineages may contain detectable errors when sequenced at high coverage. After single cell isolation, we expect the colonies to be monoclonal. For each replicate, we calculated a purity score (FIG. 13), representing the estimated fraction of plasmid molecules that are both full-length and have perfectly assembled sequences (STAR Methods). We found that on average 66% of replicates per construct had a purity score>95% (FIG. 9A, Tables S3 and S4).

### Characterization of SCRIVENER Errors

For each construct, we examined sequence features (interspersed repeats, regions of extreme GC content, regions of secondary structure) that could contribute to errors along with the read depth coverage for each replicate (FIG. 9B). Inspection of these plots revealed that the most common error mode detected was a large deletion, typically between interspersed repeats on the same DNA strand (77 of 244 assemblies with sufficient sequencing coverage contained at least one deletion, ranging from 10 to 20,616 bp). The deleted regions typically encompassed a stitching junction and involved DNA from different stitching steps, suggesting that these deletions occurred after a successful stitch. Constructs with the lowest fidelity generally contained the longest imperfect direct repeats, and deletions frequently and reproducibly occurred between these repeats. For polyclonal colonies, we often found a mixture of full-length plasmid molecules and deletion-containing molecules (incomplete dips in the coverage at deletion loci in FIG. 9B), with the fraction of deletion-containing molecules being variable, even among replicates of the same construct. For putatively monoclonal colonies that went through a single cell isolation step, we generally found that all molecules either did or did not have a deletion. However, we detected some assembly replicates (13 of 125) that still contained mixtures of plasmid molecules with and without deletions, indicating that multiple cell lineages were isolated, a single lineage maintained multiple plasmids, or mutations occurred during cell outgrowth.

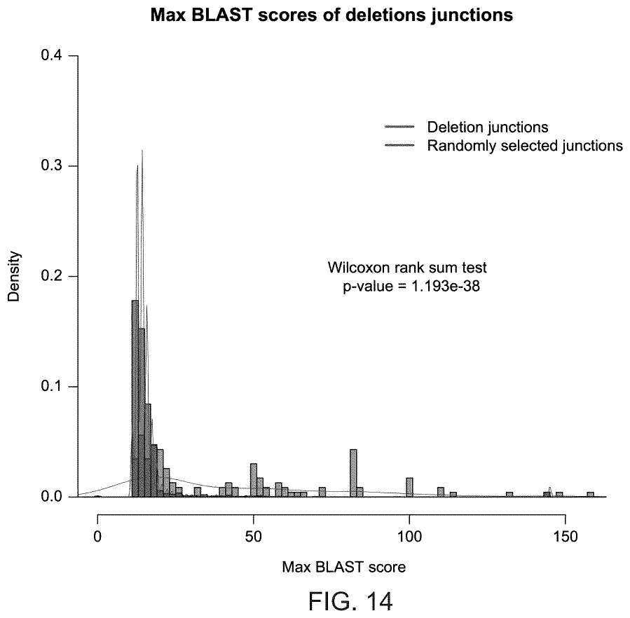

Examination of 50 bp windows flanking the predicted junction sites of the 117 detected deletions revealed a significant enrichment of long repeats in these regions (one-sided Wilcoxon rank-sum test p-value: 1.193×10⁻³⁸, FIG. 14), with 68 (59%) containing an imperfect repeat≥20 bp (20-101 bp, average of 56.7 bp and 84% sequence identity). Other deletion junctions contained only shorter regions of more perfect homology (average of 12.2 bp and 90% sequence identity), suggesting Microhomology-Mediated End Joining (MMEJ) may be a mechanism in some cases.¹⁵ In addition, we found 4 cases (1.6% of all assemblies) where the deletions coincided exactly at the DNA block boundaries. This suggests that these are not deletions but partially assembled products from previous steps that counter-selection failed to eliminate during the stitching process. Another less common error mode detected was insertions (37 insertion events in 21 assemblies ranging from 11 to 8,342 bp), 43% of which were partial duplications of either the assembled product or the plasmid backbone, and 51% of which mapped to the E. coli genome or the F-plasmid. Notably, we did not observe any point mutations caused by SCRIVENER in the assembled product of full-length plasmid molecules, although some point mutations were found in the plasmid backbones. Point mutations were commonly detected in plasmid molecules with deletions or insertions, generally near the indel junctions.

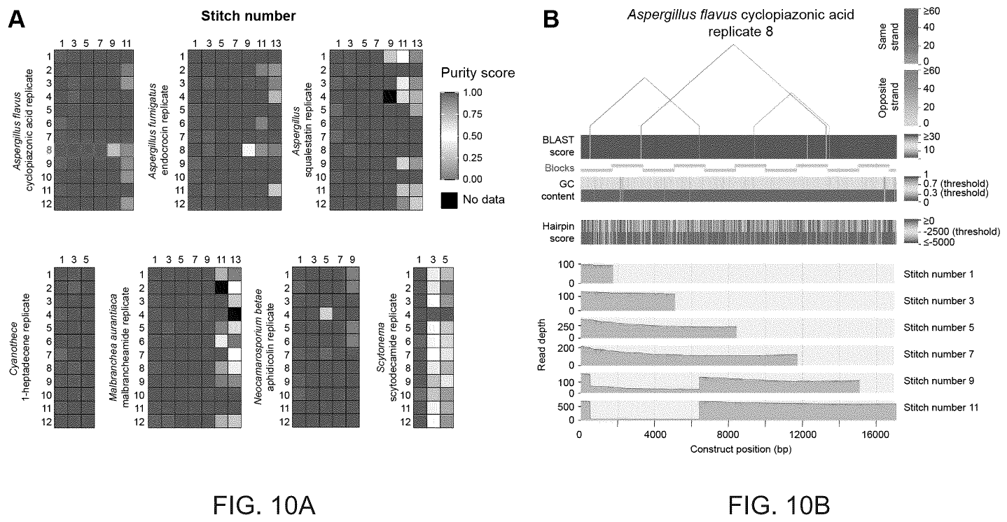

To investigate the source of deletion errors, we sequenced intermediates of the BGC assemblies at every odd stitch (stitch 1, 3, 5, etc.) and examined the assembly purity over time (FIG. 10). Read coverage plots at these intermediate stages revealed that deletions do not necessarily involve DNA actively being stitched. Instead, deletions can occur in DNA regions present early in the assembly, typically only becoming apparent later. Deletions expand in frequency over time suggesting that cells harboring these mutations are more fit, outcompeting cells with full-length products and eventually sweeping to dominate the cell population. The timing of the putative selective sweeps varied between replicates of the same assembly, suggesting that the rate at which the deletion mutation occurs and becomes established within the population is low, typically leading to stochastic, rather than deterministic, evolutionary dynamics¹⁶.

### Construction of Arrayed and Pooled Combinatorial Libraries

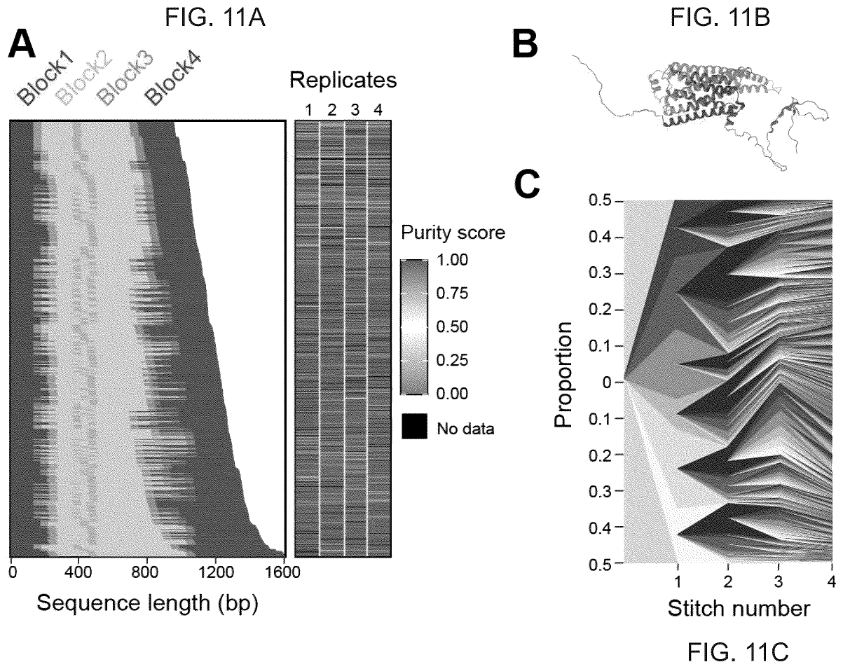

To further demonstrate the ability for SCRIVENER to scale, we next attempted to construct an arrayed combinatorial library of 1,296 G-protein coupled receptor (GPCR) chimeras. We selected six human GPCRs (ADRB2, 5HT1A, 5HT1D, 5HT4R, AA2AR, AA2BR) and broke each into four blocks (181-618 bp), with fixed homology junctions between adjacent blocks positioned in transmembrane domains (FIG. 11A, 4B,). We then assembled all 1,296 (64) combinations at 4-fold replication on 384-position agar arrays. From these 5,184 assemblies, we observed growth of all colonies on the final stitch. Following sequencing, we detected sufficient reads from 4,777 (92%) of the positions representing 1,275 (98%) of the designs, at an average replication of 3.53. Among these, 1,222 designs (94% of total) contained at least one replicate with a purity score>0.95, while 43 (3.6% of total) contained at least one replicate with sequence-perfect plasmid molecules but a lower purity score (2%-94.5%, 57.9% on average). Ten designs with sufficient sequencing coverage (0.77% of total) contained no sequence-perfect plasmid molecules in all replicates (purity score=0), possibly because they contained cytotoxic DNA sequences.

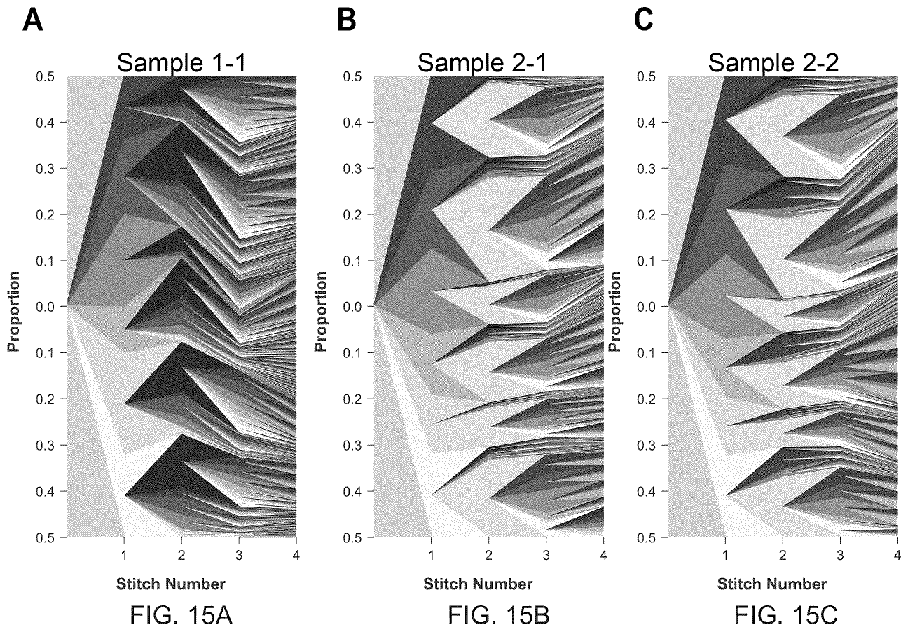

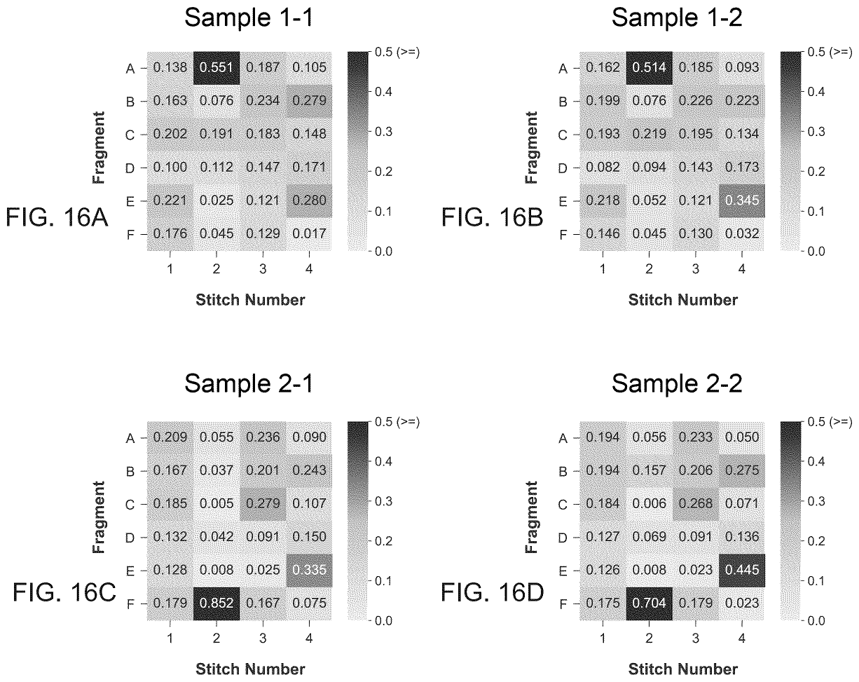

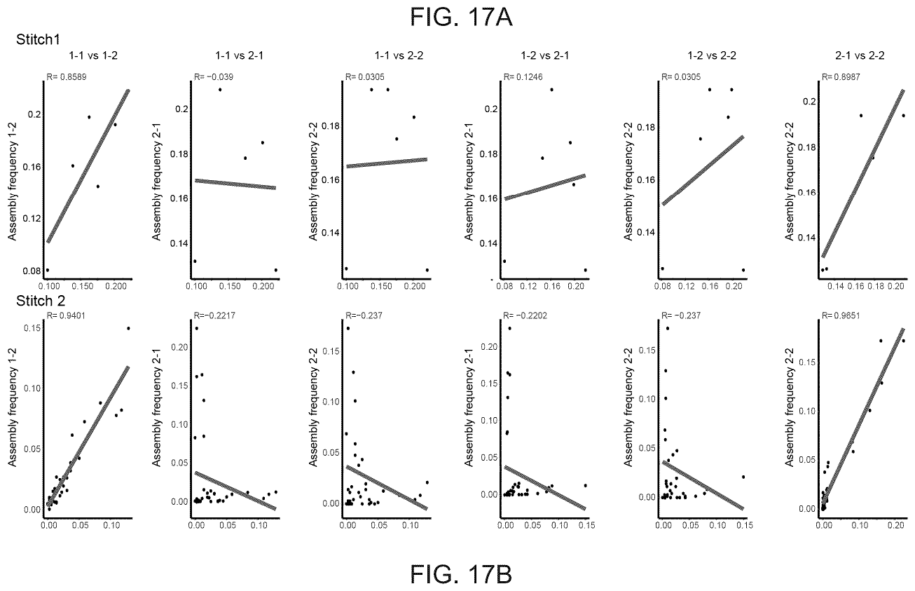

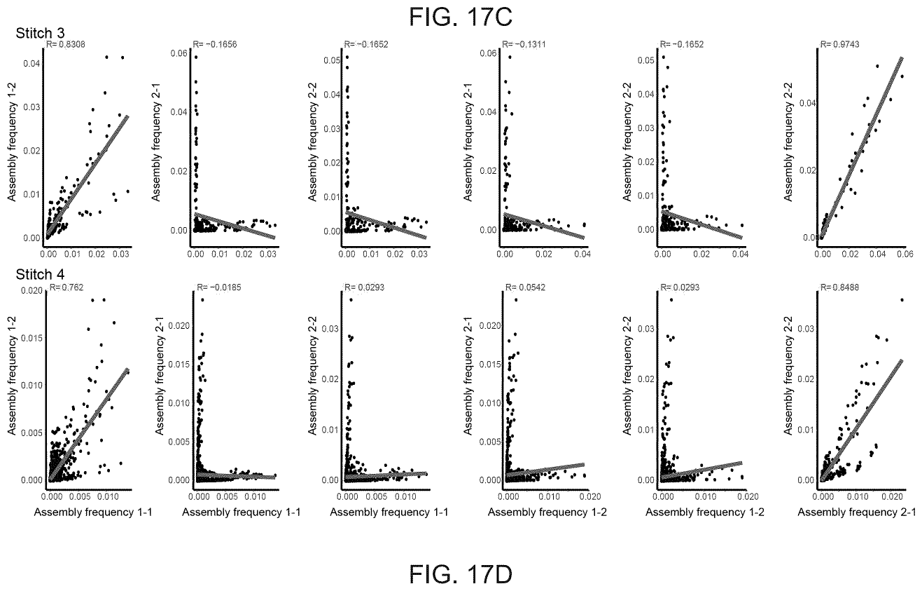

We next constructed the same combinatorial library using a simpler protocol whereby pools of donor and recipient bacteria are mated and undergo selection in 50 mL conical tubes (˜25,000 stitching events expected per cycle). We performed 4 independent pooled assemblies (2 sets of donor clones, 2 technical replicates each) and sequenced each pool at average coverage of 56, 74, 221 and 34 reads per design. We recovered 88%, 89%, 91%, and 70% of all 1,296 combinations, respectively, and detected 1,273 (98.2%) designs across the four pooled assembly replicates (FIG. 11C, FIG. 15). Examining the relative frequencies of each block in the pool revealed that some blocks were underrepresented and that frequency dispersion was likely to have a biological basis. Donor cells containing the same DNA block sometimes exhibited significantly and reproducibly different representation in the pool (FIGS. 16 and 17). These results suggest that the stitching rate can vary among biological replicates containing the identical donor plasmid sequence, a finding that was not observable during assembly in arrays with a single donor clone at each position.

To estimate the stitching accuracy of pooled assembly, we isolated and sequenced 24 colonies from each pooled assembly replicate and found that on average 75% of the colonies from each pooled assembly replicate contained a sequence-perfect design. Most errors (18 out of 24 colonies) were partially assembled products from previous assembly steps, suggesting that the counter-selection condition we chose in these pooled experiments was insufficient to remove assembly midproducts.

Reusing DNA Blocks and Assemblies without PCR

Composability, the ability to reuse and repurpose code snippets with low operational friction, has been a critical feature of information programming platforms because it accelerates the ability of engineers to build on top of each other's work^{17,18}. Repurposing of DNA blocks using existing in vitro methods generally requires that they are amplified with long primers by PCR to produce compatible ends, purified, validated, re-assembled, and transformed back into cells, a process that is difficult to automate and scale. By contrast, we reasoned that any two blocks already placed in SCRIVENER-compatible donor plasmids/cells could be seamlessly joined by utilizing small 100-140 bp “bridging” blocks that provide homology to both existing blocks. This workflow uses the same set of standardized operations as de novo DNA assembly, only some steps in the assembly process use bridging blocks. To test this idea, we attempted to join several of our previously incompatible blocks (no homology to each other) with bridges to their ends (FIG. 12A). As an added challenge, one of these assemblies contained an extremely long 2,519 bp imperfect repeat (88% sequence identity). Joining blocks with the same marker set required a single bridging block (with a different marker set), while joining blocks with different marker sets required two bridging steps. We found that the attempted bridges performed as designed, resulting in the expected chimeric assembly with high purity scores for most assembly replicates (FIG. 12A). We were also able to assemble the challenging construct containing a 2,519 bp imperfect repeat; however, it exhibited greatly reduced purity scores (0-28%), consistent with previous observations that deletions frequently occur between long repeats. As an additional validation of our purity score metric, we sequenced 12 single-colony isolates from an assembly replicate with a purity score of ˜28% and found that 4 out of 12 isolates (˜33%) contained the correct sequence.

We next asked whether a bridging block could be used to truncate an existing assembly or incoming block by providing homology to sequences in the middle, rather than the ends of these sequences. This would enable assembly of a chosen part of any existing block without PCR, thereby increasing the utility of DNA blocks already onboarded into SCRIVENER donors. We found that bridges with internal homology regions function as designed and can truncate at least 740 bp and 500 bp off of the end of a growing assembly and incoming block, respectively (FIG. 12B).

To evaluate whether bridging blocks could join already assembled products, we transferred two previously assembled constructs from recipient to donor plasmids using standard restriction enzyme cloning. Our results confirmed that these products could be seamlessly joined with a bridging block (FIG. 12C). We next evaluated the system's ability to assemble long DNA constructs using this hierarchical assembly strategy. To do so, we performed 19 sequential stitching steps, incorporating 9 linkers and 10 pre-assembled DNA blocks, to construct an ˜81.4 kb product at 96-fold replication (FIG. 12D). As the assembly length increased, we observed smaller, more sparse colonies and some colony loss after selection and counter selection, indicating a reduction in cell growth rate presumably from metabolic stress. Out of 96 assembly attempts, 74 positions produced viable colonies. We isolated 12 of these colonies and sequenced them, finding that 9 out of 12 (75%) carried no errors resulting from the assembly. Taken together, we estimate an overall assembly success rate of 57.8% for this long construct.

### DISCUSSION

We developed a high-throughput in vivo DNA assembly platform, SCRIVENER, that sequentially stitches DNA blocks together by mating bacteria in arrays or pools. SCRIVENER uses standardized protocols for onboarding of DNA blocks, mating, selection, and sequence verification, eliminating the need for idiosyncratic methods, expensive reagents (e.g., enzymes, kits), and procedures that require extended hands-on times (mixing reagents, running gels, quantitating DNA).

We show that SCRIVENER can assemble complex constructs up to 81 kb on commonly-used ColE1 vectors, but that long interspersed repeats result in stochastic deletions. Cells harboring plasmids with deletions appear to be more fit, and expand within the population over time. At long plasmid lengths, cell growth slows, presumably from metabolic stress, and assembly failures become more common. Low-copy recipient vectors, such as the Bacterial Artificial Chromosomes (BACs) provide a promising avenue by which to extend lengths further.⁸

One limitation of SCRIVENER relative to Gibson and Golden Gate assembly is that SCRIVENER blocks must be assembled sequentially while in vitro methods can assemble several blocks in one step. However, the relative simplicity and low hands-on time of SCRIVENER enables more assemblies to be processed and sequence verified in parallel. Replicates add nominal additional cost and time to process, increasing the chances of recovering a sequence perfect clone on the first try for complex assemblies. Nevertheless, in vitro assembly strategies and SCRIVENER are likely to be highly complementary. For example, Golden Gate assembly could be used to insert multiple blocks into SCRIVENER donor plasmids, which are subsequently assembled in vivo to make larger constructs. We showed that complete assemblies can be ported back into donor plasmids and used to assemble longer constructs in a hierarchical workflow (FIG. 12C, D). While we used traditional in vitro cloning methods for this example, we envision that genetic programs similar to SCRIVENER stitching could be generated to perform this DNA move operation in vivo, potentially simplifying and increasing the speed of many-part assemblies.

In our hands, the greatest bottleneck to SCRIVENER throughput is onboarding DNA blocks into the donor plasmids/cells, even when using standardized methods. However, multiple DNA synthesis providers have scaled this process and offer DNA blocks already cloned into a vector of choice. Assuming these existing processes can be modified to function with SCRIVENER donor plasmids and cells, we envision that practitioners could purchase “stitch-ready”DNA blocks in donor plasmids/cells, enabling mid- to high-throughput DNA assembly simply by mating cells.

We demonstrated that SCRIVENER blocks are composable by using short bridging blocks. This feature could be useful for quickly generating new variants as part of design-build-test cycles, for constructing higher order assemblies that concatenate two or more small assemblies, and for shuffling the order of an assembly to optimize transcription or other properties. Bridging blocks can be designed to provide homology to the middle of a growing assembly or incoming block, thereby enabling selected parts of existing blocks to be reused in high throughput without a PCR step. While our initial tests indicated that bridges can truncate up to 740 bp off of a growing assembly and 500 bp off of an incoming block, further work is needed to assess practical truncation length limits. Depending on these results, it may be valuable to construct arrayed SCRIVENER donor cell libraries that create a reservoir of useful DNA sequences that can be reused without PCR (e.g., a human cDNA library). Composability of the SCRIVENER platform could incentivize DNA block storage, reuse, and sharing, facilitating the development of a robust disaggregated DNA engineering ecosystem where practitioners can build on top of each other's designs¹⁹. Instantiating such a DNA engineering environment requires products that further simplify SCRIVENER engineering (e.g. assembly kits and stitch-ready DNA blocks) and ecosystem infrastructure investments, such as those that increase the capacity of plasmid repositories or develop a SCRIVENER-compatible DNA block marketplace.

Data and Code availability: GenBank files of all plasmid backbones and a table of DNA blocks used in this study are below or available in the Supplementary data with Matsui et al. 2024 bioRxiv preprint available at //doi.org/10.1101/2024.09.03.611066 High-throughput DNA engineering by mating bacteria. The raw FASTQ files from Oxford Nanopore sequencing conducted in this study are available in the Sequence Read Archive (SRA BioProject PRJNA1198116). Code for sequence and data analysis is available in the GitHub repository github.com/tmatsui22222/SCRIVENER.

Genbank files of plasmids used in this study are shown in the sequence listing.

TABLE 1
Estimated costs for different steps in an in vivo DNA assembly
workflow. SCRIVENER HT is assembly on agar. SCRIVENER
MT is assembly in liquid media in 96-well plates.

| SCRIVENER | — | — | — | — |
|---|---|---|---|---|
| HT | | | | |
| Item | Unit cost | Cost | Cost per | Notes |
| | | per | base | |
| | | stitch | (1.8 kb | |
| | | | blocks) | |
| Singer | $2.18 | $0.02 | $0.000013 | 4 plates at 384 |
| PlusPlate | | | | positions/plate (donor plate, |
| | | | | mating plate, selection plate, |
| | | | | counter-selection plate) |
| Singer Repads | $0.91 | $0.01 | $0.000005 | 4 384-pin pads |
| LB Agar | $580.00 | $0.01 | $0.000004 | Assume 4 100 mL plates at |
| (2.5 kg) | | | | 384 positions/plate, |
| | | | | additional cost for selection |
| | | | | drugs is negligable |
| Consumables | | $0.04 | $0.000022 | |
| Subtotal | | | | |
| Equipment | $35,000.00 | $0.09 | $0.000050 | Assume 1536 stitches per |
| (Singer | | | | work day |
| ROTOR | | | | |
| amortized cost | | | | |
| per year) | | | | |
| Labor (cost per | $50.00 | $0.06 | $0.000034 | Assume 2 minutes to pour |
| hour) | | | | an agar plate, and 5 minutes |
| | | | | for each pinning step |
| Equipment | | $0.15 | $0.000084 | |
| and Labor | | | | |
| Subtotal | | | | |
| TOTAL | | $0.19 | $0.000106 | |
| SCRIVENER | — | — | — | — |
| MT | | | | |
| Item | Unit cost | Cost | Cost per | Notes |
| | | per | base | |
| | | stitch | (1.8 kb | |
| | | | blocks) | |
| 96-well Plate | $4.70 | $0.20 | $0.000109 | 4 plates at 96 positions/plate |
| | | | | (donor plate, mating plate, |
| | | | | selection plate, counter- |
| | | | | selection plate) |
| Pipette tips | $27.00 | $0.11 | $0.000063 | Assume 4 tips/stitch for |
| (960) | | | | mating and selection |
| LB (2.5 kg) | $403.00 | $0.002 | $0.000001 | Assume 4 96-well plates at |
| | | | | 10 mL/plate |
| Consumables | | $0.31 | $0.000172 | |
| Subtotal | | | | |
| Equipment | $0.00 | $0.00 | $0.000000 | |
| (amortized cost | | | | |
| per year) | | | | |
| Labor (cost per | $50.00 | $0.36 | $0.000199 | Assume 30 minutes to make |
| hour) | | | | media, 5 minutes to fill a |
| | | | | plate and 5 minutes for each |
| | | | | plate transfer step |
| Equipment | | $0.36 | $0.000199 | |
| and Labor | | | | |
| Subtotal | | | | |
| TOTAL | | $0.67 | $0.000371 | |
| MULTIPLEX | — | — | — | — |
| ED WHOLE- | | | | |
| PLASMID | | | | |
| SEQUENCE | | | | |
| VERIFICATION | | | | |
| Item | Unit cost | Cost | Cost per | Notes |
| | | per | base | |
| | | plasmid | (10 kb | |
| | | | constructs) | |
| QIAprep Spin | $497.00 | $0.01 | $0.000001 | Assume 384 barcoded |
| Miniprep Kit | | | | plasmids per miniprep |
| (250 preps) | | | | |
| Oxford | $999.00 | $0.02 | $0.000002 | Assume 24 ONT barcodes |
| Nanopore | | | | used per flow cell, 384 |
| Rapid | | | | plasmids per ONT barcode |
| Barcoding Kit | | | | |
| 96 V14 | | | | |
| MinION Flow | $900.00 | $0.10 | $0.000010 | Assume 9,216 plasmids per |
| Cell (R10.4.1) | | | | flow cell at >100X coverage |
| Consumables | | $0.12 | $0.000012 | |
| Subtotal | | | | |
| Equipment | $0.00 | $0.00 | $0.000000 | Negligable amortized cost |
| | | | | for MinION |
| Labor (cost per | $50.00 | $0.02 | $0.000002 | Assume 4 hours to process |
| hour) | | | | 9,216 positions in parallel |
| Equipment | | $0.02 | $0.000002 | |
| and Labor | | | | |
| Subtotal | | | | |
| TOTAL | | $0.14 | $0.000014 | |

TABLE 2
Plasmids used in this study

| | | | SEQ |
|---|---|---|---|
| systematic | description | GeneBank file name in | ID |
| name | name | Supplemental Data 3 | NO: |
| eBSD28 | helper | eBSD28_helper_plasmid.gb | 2 |
| | plasmid | | |
| eBSD10 | recipient | eaBSD10_recipient_plasmid.gb | 3 |
| | plasmid | | |
| eBSD13 | 1st odd | eBSD13_1st_odd_donor_plasmid.gb | 4 |
| | donor | | |
| | plasmid | | |
| eBSD16 | odd donor | eBSD16_odd_donor_plasmid.gb | 1 |
| | plasmid | | |
| eBSD19 | even donor | eBSD19_even_donor_plasmid.gb | 5 |
| | plasmid | | |
| eBSD22 | even donor | eBSD22_even_donor_plasmid.gb | 6 |
| | plasmid | | |

### STAR Methods

Strains Used in this Study

The donor strain (dSL2: HB101 ΔuidA::pir+, ΔendA::FRT, F128-xx (oriT::TcR)) and recipient strain (rSL2: HB101 ΔendA::FRT) were constructed from the HB101 strain²¹ (araC14, leuB6(Am), Δ(gpt-proA)62, lacY1, glnX44(AS), galK2(Oc), λ-, recA13, rpsL20(strR), xylA5, mtl-1, thiE1, [hsdS20]) using a pop-in pop-out strategy, as described²². The endA gene was deleted in both donor and recipient strains to increase plasmid stability. Insertion of pir+ gene in the genome of the donor strain was necessary to support replication of the donor plasmids with an R6Kγ origin of replication²³. The oriT in the F128-xx plasmid was replaced with a tetracycline resistance marker (TcR) to prevent conjugation of the F plasmid itself to the recipient strain.

### Media and Chemicals

Luria-Bertani (LB) broth was used for cloning and for growth of donor and recipient plasmids. For selection and maintenance plasmids, antibiotics were added at the following concentrations: kanamycin (Kan) (25 μg/mL), spectinomycin (Sp) (60 μg/mL), hygromycin B (Hyg) (100 μg/mL), gentamicin (Gm) (25 μg/mL), tetracycline (Tc) (2 μg/mL), carbenicillin (Carb) (100 μg/mL), chloramphenicol (Cm) (25 μg/mL), apramycin (Apm) (100 μg/mL), streptomycin (Str) (25 μg/mL). For counter-selection and removal of plasmids, 6% sucrose was added for SacB, and 200 μg/mL 4-chloro-phenylalanine (4CP) was added for PheS. To improve the efficiency of PheS counterselection in LB, T251A/A294G ePheS variant was used²⁴. L-arabinose (0.2% w/v) and L-rhamnose (0.2% w/v) were used to induce the ParaBAD and PrhaBAD promoters, respectively.

Plasmids Used in this Study

All plasmid backbones used in this study were constructed by restriction digestion and ligation or Gibson assembly of synthetic gene blocks sourced from Twist, IDT, or GenScript and are listed in Table 2.

### Construction of Barcoded Recipient Cell Arrays

To generate arrays of recipient strains with uniquely barcoded recipient plasmid, the rSL2 recipient strain was transformed with helper plasmid eBSD28 and selected for integration using LB+100 μg/mL Carb to create eBSD35. The helper plasmid eBSD28 contains an arabinose inducible Cas9, rhamnose inducible lambda red recombineering genes, and a temperature-sensitive pSC101 origin. Barcoded recipient plasmids were constructed by inserting a random 20mer into the eBSD10 plasmid backbone region. To achieve this, eBSD10 was first PCR amplified using 2 pairs of primers: oSL1482 (which contains the 20mer barcode) and oSL1484, and oSL1426 and oSL1483. The PCR amplified products were gel extracted and assembled via NEBuilder HiFi DNA assembly master mix (NEB). The assembled product was transformed into eBSD35, selected on LB+25 μg/mL Gm, and 672 single colonies were picked. The barcode region from presumptive clones was amplified using primers oBSD20 and oBSD9 and Sanger sequenced. Unique barcodes with a Levenshtein distance greater than 6 to each other were selected and rearrayed to generate three separate arrays of barcoded recipient clones (paSL5—96 barcodes, paSL6—384 barcodes, and eaBSD3—96 barcodes, 576 barcodes total).

Insertion of DNA Blocks into Donor Plasmids

DNA blocks used for assemblies were synthesized by Twist Bioscience (Document S2), IDT, or amplified from BY4716 yeast genomic DNA by PCR via PrimeSTAR or Platinum SuperFi II DNA Polymerase (Thermo Fisher Scientific). The blocks were cloned into linearized donor vectors by standardized methods using either restriction enzyme cloning, Gibson cloning, or gap repair cloning. For restriction enzyme cloning, the DNA block and the appropriate donor plasmid were digested by the restriction enzymes AscI and NotI and purified by gel extraction. The purified plasmid backbone and insert was ligated by T4 ligase reaction following the standard protocol (NEB). The ligated products were transformed into the dSL2 donor cell and selected on the appropriate LB+antibiotic plates.

For Gibson cloning, the appropriate linearized plasmid and purified DNA block was mixed into the NEBuilder HiFi DNA assembly master mix following standard protocol (NEB) and then transformed into dSL2 donor cells. Overlaps of 20-25 bp were used.

For gap repair cloning, 200 ng of the DNA block and 200 ng of the appropriate linearized donor plasmid were transformed into dSL2 donor cells to assemble the two fragments via the endogenous E. coli machinery²⁵. Overlaps of 20-25 bp were used.

All cloned plasmids were purified using a Qiagen miniprep. For small inserts, insertion regions were verified by Sanger sequencing (McLAB or Azenta/Genewiz). For larger inserts, whole plasmids were sequenced by Oxford Nanopore sequencing (Plasmidsaurus, Primordium, or in-house).

### Testing DNA Stitching Fidelity, Efficiency, and Homology Requirements for SCRIVENER

The mPapaya fluorescence gene was split into four DNA blocks such that four stitches are necessary to complete the assembly. All blocks were inserted into the appropriate donor plasmid/strain (eBSD13, eBSD16, or eBSD19 in strain dSL2) The first three DNA blocks were stitched into recipient strain rSL2 and tests were performed by stitching the fourth DNA block. Several mPapaya fourth donors were designed such that the donor blocks have 0, 10, 20, 30, 40, 50, and 60 bp of homology with the third mPapaya stitch product. The recipient strains and the different donor strains were first grown overnight in LB+60 μg/mL Sp+25 μg/mL Gm+100 μg/mL Carb and LB+100 μg/mL Hyg+2 μg/mL Tc, respectively. The cells were then diluted 100 fold with the same LB+antibiotic and grown at 30° C. for four hours. After growth, OD600 was measured for each culture, and each culture was then normalized to OD600 of 0.5. Twenty-five μL of the recipient strain was mixed with 75 μL of one of the donor strains, with three replicates for each recipient-donor pair. The cell mixtures were centrifuged and re-suspended in 1 mL of SOB+0.2% arabinose+0.2% rhamnose and grown at 30° C. for two hours. After growth, OD600 was measured again for each culture to make sure a similar number of cells were being plated after mating across all conditions. Fifty μL of the culture was plated onto LB+50 μg/mL Sp+25 μg/mL Gm+100 μg/mL Carb agar plates and grown for two days at 30° C. The cells were then replica-plated using a velvet onto LB+50 μg/mL Sp+25 μg/mL Gm+100 μg/mL Carb+6% sucrose agar plates and grown at 30° C. for one day. The total number of colonies and the number of fluorescent colonies was counted for each and used for calculation of relative stitching fidelity and efficiency.

To estimate the absolute stitching efficiency for the stitch with 50 bp of homology, we first determined the colony-forming units (CFU) in a 1 mL culture at an OD600 of 1 for both the recipient and donor strains. A recipient strain carrying the third mPapaya stitch product and a donor carrying the fourth mPapaya DNA block with 50 bp homology were grown overnight in LB+60 μg/mL Sp+25 μg/mL Gm+100 μg/mL Carb and LB+100 μg/mL Hyg, respectively. They were then diluted 100 fold and grown at 30° C. for four hours. After growth, OD600 was measured for each replicate and each culture was normalized to either OD600 of 0.25, 0.5, and 0.75, with three replicates each. The normalized cultures were further diluted to 10⁻⁵ in 1 mL of SOB, and 100 μL of this dilution (for a total of 10⁻⁶ dilution) was plated onto either LB+60 μg/mL Sp+25 μg/mL Gm+100 μg/mL Carb agar plate for recipient strains and LB+100 μg/mL Hyg agar plates for donor strains. The cells were grown for two days at 30° C. and the total number of cells was counted. To get the CFUs per 1 mL when OD600 equals 1, the number of colonies was multiplied by 10⁶*(1/initial OD600). On average, a CFU was ˜1.5×10⁸/mL for donor strains and ˜3×10⁸/mL for recipient strains. Using this CFU value for the recipients, stitching efficiency was determined according to the following equation: efficiency=((number of colonies after counter-selection))/((OD600 before mating/OD600 after mating)×(recipient CFU per 1 mL when OD600 equals to 1)×(volume used for mating in μL/1,000 μL/mL)). The efficiency measured for each replicate was 9.05×10⁻⁶, 1.23×10⁻⁵, and 1.04×10⁻⁵, for an average efficiency of 1.06×10⁻⁵. These estimates are conservative because they assume that all stitching happens immediately upon mating and all outgrowth following mating is creating replicates of successful stitching events. In reality, some outgrowth is happening before stitching, which, if measurable and taken into consideration, would increase the efficiency we calculated.

### Arrayed Assembly on Agar Plates

Arrayed assemblies were performed in an 96- or 384-position format by mating arrays of donor strains with an arrays of barcoded recipient strains, then pinning to selectable agar pads and counter-selectable agar pads in series using a Singer ROTOR HDA pinning robot. Recipient arrays were grown at 30° C. overnight on LB+25 μg/mL Gm+100 μg/mL Carb, and the donor arrays were grown at 37° C. overnight on LB+100 μg/mL Hyg+2 μg/mL Tc. The donor and recipient colonies were then pinned and mixed onto the same mating plate (LB+0.2% arabinose+0.2% rhamnose) and incubated at 30° C. for 4-6 hours. To select for recombinant recipient cells, the mated colonies were pinned onto a selection plate LB+100 μg/mL Hyg+25 μg/mL Gm+100 μg/mL Carb+0.2% arabinose and incubated at 30° C. overnight. The next day, cells on the selection plate were pinned to the counter-selection plate LB+100 μg/mL Hyg+25 μg/mL Gm+100 μg/mL Carb+200 μg/mL 4CP and incubated at 30° C. overnight, completing the first stitch. In parallel, donor cells for the second stitch were grown at 37° C. overnight on either LB+60 μg/mL Sp+2 μg/mL Tc plate or LB+100 μg/mL Apm+2 μg/mL Tc plate.

To initiate the second stitch, recombinant recipients from the first stitch and donors for the second stitch were pinned and mixed onto the same mating plate (LB+0.2% arabinose+0.2% rhamnose) using a Singer ROTOR and incubated at 30° C. for 4-6 hours. To select for only the recombinant recipient cells, the mixed cells were then pinned onto either an LB+60 μg/mL Sp+25 μg/mL Gm+100 μg/mL Carb+0.2% arabinose plate or LB+100 μg/mL Apm+25 μg/mL Gm+100 μg/mL Carb+0.2% arabinose plate, depending on the the selection marker in the donor cells. After overnight growth at 30° C., cells on the selection plate were pinned to either an LB+60 μg/mL Sp+25 μg/mL Gm+100 μg/mL Carb+6% sucrose plate or LB+100 μg/mL Apm+25 μg/mL Gm+100 μg/mL Carb+6% sucrose plate and incubated at 30° C. overnight, completing the second stitch. In parallel, donor cells for the third stitch were grown at 37° C. overnight on LB+100 μg/mL Hyg+2 μg/mL Tc plate.

The assembly process and agar plates used for every odd stitch onwards (third, fifth, seventh, etc.) is the same as the first stitch, and the assembly process and agar plates used for every even stitch onwards (fourth, sixth, etc.) is the same as the second stitch. Following the last round of assembly, the helper plasmid with the temperature-sensitive origin was removed from the recipient cells by transferring the cells to an extra round of counter-selection plates without 100 μg/mL Carb and growing them at 37° C. overnight.

### Arrayed Assembly in Liquid

Arrayed assemblies were conducted in liquid in 96-well plates. The strains, media, and antibiotics used for each stitching step are the same as those in the agar assembly described above. To initiate the stitching process in a 96-well plate, the barcoded array of recipient strains and donor strain array were grown in 150 μL of LB+25 μg/mL Gm+100 μg/mL Carb, and LB+100 μg/mL Hyg+2 μg/mL Tc, respectively, on an orbital plate shaker at 37° C. overnight with 225 rpm shaking. Then, 120 μL of the donor cells were mixed with 30 μL of recipient cells and centrifuged. The supernatant was removed using a multi-channel pipette, and the cells were resuspended in 100 μL of LB+0.2% arabinose+0.2% rhamnose. The mixed cells were mated for 4-6 hours at 30° C. with 225 rpm shaking. To select for the recombinant recipient cells, 15 μL of the mixed cells were transferred into 135 μL of LB+100 μg/mL Hyg+25 μg/mL Gm+100 μg/mL Carb selection media and grown overnight at 30° C. with 225 rpm shaking. The next day, 5 μL of the overnight culture was transferred into 145 μL of LB+100 μg/mL Hyg+25 μg/mL Gm+100 μg/mL Carb+200 μg/mL 4CP counter-selection media and grown overnight at 30° C. with 225 rpm shaking. In parallel, donor cells for the second stitch were grown at 37° C. overnight with 225 rpm shaking in 150 μL of either LB+60 μg/mL Sp+2 μg/mL Tc or LB+100 μg/mL Apm+2 μg/mL Tc.

To initiate the second stitch, 30 μL of recombinant recipients from the first stitch and 120 μL of donors for the second stitch were mixed and centrifuged. The supernatant was removed using a multi-channel pipette, and the cells were resuspended in 100 μL of LB+0.2% arabinose+0.2% rhamnose. The mixed cells were mated for 4-6 hours at 30° C. with 225 rpm shaking. To select for the recombinant recipient cells, 15 μL of the mixed cells were transferred into 135 μL of either LB+60 μg/mL Sp+25 μg/mL Gm+100 μg/mL Carb or LB+100 μg/mL Apm+25 μg/mL Gm+100 μg/mL Carb selection media and grown overnight at 30° C. with 225 rpm shaking. The next day, 5 μL of the overnight culture was transferred into 145 μL of either LB+60 μg/mL Sp+25 μg/mL Gm+100 μg/mL Carb+6% sucrose or LB+100 μg/mL Apm+25 μg/mL Gm+100 μg/mL Carb+6% sucrose counter-selection media and grown overnight at 30° C. with 225 rpm shaking. In parallel, donor cells for the third stitch were grown at 37° C. overnight with 225 rpm shaking in 150 μL of LB+100 μg/mL Hyg+2 μg/mL Tc.

The assembly process and media used for every odd stitch onwards (third, fifth, seventh, etc.) are the same as the first stitch, and the assembly process and media used for even stitch onwards (second, fourth, sixth, etc.) are the same as the second stitch. Following the last round of assembly, the helper plasmid with the temperature-sensitive origin was removed from the recipient cells by transferring 5 μL of cells to an extra round of 145 μL of counter-selection media without 100 μg/mL Carb and growing them at 37° C. overnight.

### Design of the GPCR Variant Library

We aimed to create a library of novel G protein-coupled receptor (GPCR) chimeras for high-throughput ligand screening in engineered yeast cells. Utilizing GPCRdb, we performed a multiple sequence alignment of 56 human GPCRs that are functional in yeast^{20,26,27}. By applying a 15-amino acid (45-bp) percent identity sliding window average over the alignment and excluding positions with gaps, we identified three local maxima in transmembrane helices 2, 4, and 6. These local maxima served as fixed homology regions for our assemblies, resulting in the creation of four DNA blocks per gene.

To minimize mutations in the three 45-bp homology regions, gene selection was prioritized based on higher percent identity in these regions from the initial alignment. For future library use in yeast, we selected GPCRs with hydrophilic ligands, closely related family members, and those with available structural and pharmacological data. The chosen receptors included two adenosine receptors (ADORA2A, ADORA2B), three serotonin receptors (HTR1A, HTR1D, HTR4), and an epinephrine receptor (ADRB2) (Table S7). The amino acid sequences were codon optimized for Saccharomyces cerevisiae using Integrated DNA Technology's Codon Optimization Tool (idtdna.com/CodonOpt).

### Pooled Assembly in Liquid

We performed four independent pooled assemblies (two technical replicates of two biological replicates using different donor colones with the same DNA block sequences). Cell pools were grown in standard 50 mL conical tubes. For each stitch, 33 OD of donor cells (5.5 OD for each donor strain) and 8.25 OD of recipient cells from the previous stitch were mixed and spun down at 3,900 rpm for 10 minutes. The cell pellets were suspended in 10 mL LB+0.2% arabinose+0.2% rhamnose medium and incubated at 30° C. for 4-5 hours with 250 rpm shaking. Based on an estimated stitching efficiency of 10⁻⁵ calculated above, we expected ˜2.5*10⁴ stitching events in the pool (2.48*10⁹ cells/10⁻⁵) or ˜20× the number of designs. Cell cultures were spun down, resuspended in 30 mL of selection medium (first stitch and odd stitch selection: LB+200 μg/mL Hyg+25 μg/mL Gm+100 μg/mL Carb+0.2% arabinose; even stitch selection: LB+60 μg/mL Sp+25 μg/mL Gm+100 μg/mL Carb+0.2% arabinose), and incubated at 30° C. overnight with 250 rpm shaking. Next, 15 mL of selected cells were spun down and resuspended with 45 mL of the same selection medium for 5 hours at 30° C. with 250 rpm shaking. For counter-selection, 5 mL of this culture was mixed with 45 mL counter selection medium (first stitch and odd stitch counter-selection: LB+200 μg/mL Hyg+25 μg/mL Gm+100 μg/mL Carb+200 μg/mL 4CP; even stitch counter-selection: LB+60 μg/mL Sp+25 μg/mL Gm+100 μg/mL Carb+6% sucrose) at 30° C. overnight with 250 rpm shaking. Donor cells for the following stitch were cultured on the same day at 37° C. overnight with 250 rpm shaking. Following the last round of assembly, the helper plasmid with the temperature-sensitive origin was removed from the recipient cells by doing an extra round of counter-selection without 100 μg/mL Carb and incubated at 37° C. overnight.

### Data Analysis for Pooled GPCR Assembly

We sequenced the pool of GPCR assembly products at every stitch by Oxford Nanopore Sequencing and mapped each read to the 1,296 GPCR designs using minimap2 (v2.28) using minimap2-c options to generate paf file²⁸. The paf files provided the mapped reference and the number of residue matches for each read. Reads that mapped to multiple GPCR variants were assigned to the assembly product with the highest number of residue matches. Reads that matched equally to two assembly products were not counted. Table S8 records the number of reads that mapped to each design before and after filtering. Some block combinations that were not detected by sequencing at early stages of the assembly were detected at a later stage of the assembly, indicating the sequencing data missed some designs present in the pool. Python package muller v0.6.0 is used for making Muller plots²⁹.

Sequence Verification of Single Colonies from Pooled GPCR Assembly

Based on the frequency of stitching in liquid of 10⁵, we expected that each assembly was the product of multiple assembly lineages, some of which may contain an error. Because of the high error rate of Oxford Nanopore sequencing (Q18-20), we could not determine if an error of given read was due to a sequencing error or a true error in a lineage. To more accurately determine the stitching fidelity of the pooled assembly, the four pooled GPCR assemblies were streaked onto LB+100 μg/mL Sp+25 μg/mL Gm agar plates and grown overnight at 37° C. From each replicate, 24 colonies were picked, and plasmids were extracted using the Qiagen MiniPrep kit. The plasmids were then individually sequenced (Plasmidsaurus/Primordium). Alignments (minimap2) between the consensus sequences generated by Plasmidsaurus/Primordium and the expected reference sequences were used to estimate the error rate.

### Examination of Sequence Features in Constructs

For each construct assembled in this paper, several sequence features were examined: interspersed repeats, extreme GC content, and secondary structures. To search for interspersed repeats, BLAST (v2.16.0) “blastn” function was used to align the construct sequence to itself with the following parameters: -word_size 6, -reward 1, -penalty -1, -gapopen 3, -gapextend 2, -evalue 5, and -perc_identity 70. To identify regions with extremely high or low GC %, the GC % in a 51 bp rolling window was calculated. To find any secondary structures, Python package “Primer3” (primer3-py v2.0.3) was used with default parameters except for hairpin_window_size, which was changed to 41.

### Choosing Regions of Homology Between DNA Blocks

DNA blocks for each construct, along with their corresponding homology regions, were manually designed while taking into consideration specific sequence features that may affect homologous recombination efficiency and fidelity, as described above. For fluorophores, yeast chromosomes, and synthetic DNA, variable-sized DNA blocks were created to ensure that the homology regions had minimal repetitive sequences, homopolymers, and secondary structures, while maintaining a GC content close to 50%. For BGCs, all DNA blocks were ordered as 1,800 bp fragments. If homology regions contained repetitive sequences, homopolymers, secondary structures, or extreme GC content, the length of the homology region was extended up to 100 bp, depending on the extent of these sequence features.

### Oxford Nanopore Technologies (ONT) Sequencing and Data Analysis

For high-throughput in-house sequencing, arrays of cells containing barcoded recipient plasmids (with barcodes marking position on a plate) were pooled by adding 10 mL of water to each agar plate and scraping the cells using a cell spreader. Since the plasmid size can vary significantly depending on the assembly (ranging from ˜6.5 kb to ˜27 kp) and size may impact the plasmid prep efficiency, we designed assembly plates such that all positions contained plasmids within a 1 kb range of each other. Any plates with overlapping barcodes or large differences in plasmid size were pooled separately. After pooling, the plasmids were extracted from the harvested cells using Qiagen Miniprep kits.

Sequencing libraries for each plasmid pool were prepared using the Rapid Barcoding Kit 96 V14 (SQK-RBK 114.96) and run on Nanopore MinION Flow Cell (R10.4.1; FLO-MIN114). Each plasmid pool that was run on the same flow cell was uniquely barcoded with a different Rapid Barcode. The libraries were prepared using the standard protocol, with the following modifications: 1) 200 ng of input DNA was used instead of 50 ng, 2) 1.5 μL of the Rapid Barcode reagent was used instead of 1 μL, 3) DNA was eluted from the AMPure XP beads using 15 μL of Elution Buffer EB per 24 Rapid Barcodes instead of a flat 15 μL, and 4) up to 2 μg of a DNA library was loaded onto the flow cell instead of the recommended maximum of 800 ng. For each sequencing run on a flow cell, we aimed for at least 100× coverage per plasmid. To minimize bias in sequencing due to plasmid size, only plasmid pools within a 5 kb range of each other were run together on the same flow cell. Data from the flow cell was basecalled using Dorado (https://github.com/nanoporetech/dorado) with Super-accuracy basecalling. The data were then demultiplexed by Rapid Barcodes using Dorado.

For runs with a small number of plasmids, the flow cells were stopped when the number of reads per rapid barcode reached ˜200 times the number of plasmids. The flow cells were then washed using the Flow Cell Wash Kit (EXP-WSH004) following the provided protocol before loading the next set of DNA libraries.

ONT reads were analyzed by custom developed software in Python and executed on a local 36-core cluster. The pipeline begins with a demultiplexing step where sequencing reads are separated based on custom barcodes in the recipient plasmid using a tailored demultiplexing program. Following demultiplexing, the reads are aligned to reference sequences with minimap2 (v2.28) using the “map-ont” option^{28,30}. The alignment files are sorted and indexed using samtools (v1.20) using default parameters³¹. Variant calling is performed using bcftools³¹ (v1.20) with the mpileup (default ont-sup-1.20 parameters, except the coefficient for modeling homopolymer errors to 200, and max-depth to 10,000), and call (default parameters, except ploidy is set to 1) commands to identify small mutations and indels. Large structural variants (indels>10 bp) are identified with Sniffles2 (v2.2) using default parameters³².

To estimate the purity score, which represents the fraction of plasmid molecules with correctly assembled full-length products within an assembly replicate, read lengths were extracted from the demultiplexed FASTQ files. A histogram with a bin size of 50 bp was then created. “Peaks” or enrichment of certain read lengths, which presumably represents sequence data from fully intact plasmid DNA molecules that were not fragmented during plasmid extraction and library preparation, were identified using SciPy (v1.14.0) “find_peaks” function, with several changes from the default parameters. To account for the difference in read coverage, different values were used for the height and prominence parameters. The height parameter was set to 8, or to 5% of the number of reads in the largest bin (most number of reads), whichever is higher. The prominence parameter was set to 3.1, or two times the standard deviation in number of reads across all bins, whichever is higher. The distance parameter was set to two. After the peaks were detected, reads were assigned to the nearest peak to form clusters. Any reads farther than 150 bp away from a cluster peak (presumably caused by partial sequencing reads from fragmented DNA during library preparation) were not assigned to a cluster. The purity estimation of each sample was calculated by determining the proportion of reads clustered within 150 bp of the expected plasmid size over all reads assigned to any cluster. The reference mapping and variant calling analysis is reiterated on clustered reads to refine the variant calls and QC assessments. Only point mutations called with high confidence (combined quality score>100) were output in the final QC report generated by the nanopore sequencing analysis pipeline. Any identified mutations and indels were categorized based on whether they were in the assembled product or the plasmid backbone.

We next adjusted the purity scores calculated above to only consider errors that impact the assembly fidelity. For the read cluster of the expected size, if any point mutations or indels were found in the assembly sequence, the purity score was assigned to zero, with the exception of point mutations that could be traced back to being present in the donor plasmids/cells. Mutations found in donor plasmids were ignored, since these mutations do not reflect assembly errors and are likely to stem from errors in synthesis. Mutations or deletions<100 bp that were detected in the plasmid backbone were also ignored. We rarely encountered instances where large structural rearrangements (indels>100 bp) in the plasmid backbone resulted in a lower purity score by forming a separate read length cluster, despite having a sequence-perfect assembly region. In these cases, we did not adjust the purity score, as the large indels could indicate plasmid instability and an undesirable plasmid product . . . .

The identities of insertions called by Sniffles2 (v2.2) (N=37) were mapped using minimap2 with default parameters against the sequences of the donor and recipient plasmids, and the sequences of the donor strain genome and F-plasmid.

For replicate assemblies of the three fluorophores (mPapaya, mPlum, sfGFP, 4 stitches each), we assumed a replicate contained no errors if >95% of molecules are sequence perfect. Errors in these assemblies were caused by a subpopulation of molecules with point mutations or undesired assembly products. For other assemblies performed here, we reported the purity score directly and discussed errors in detail in the Results section.

### Sequence Verification of the 81 kb Assembly Product

Because of its long length, most nanopore sequencing reads did not encompass the full-length of the plasmid. Because a barcode at a single location on the plasmid is required to demultiplex pooled reads, our typical pooled sequencing pipeline could not be used here. To validate these assemblies, we instead isolated 12 single colonies, extracted plasmids independently for each, and submitted them for sequencing at Plasmidsarus. To identify variants, we aligned consensus sequences to the reference using MAFFT v7.526 with parameters --adjustdirection --add consensus_sequence_from_plasmidsarus reference_sequence, then extracted insertion, deletion, and mutation data from the alignment file using custom Python code.³³ Clones from C2, C5, C6, C7, and C12 were a perfect sequence following Oxford Nanopore sequencing. Clones C1, C3, and C4 each contained unique errors, indicating errors in assembly. Clones C8 and C11 contained an apparent error in a homopolymer region by Oxford Nanopore sequencing, but were then verified to be sequence perfect by Sanger sequencing. Clones C9 and C10 contained a common variant at position 49,384, indicating that this was an error in the donor plasmid sequence, not an assembly error. In total, 9 out of 12 replicates contained no assembly errors.

### GPCR Protein Structure

The predicted protein structure for a GPCR was generated in AlphaFold using the human GPCR variant ADRB2, which is one of the GPCRs in our test set (only it has been modified at the homology regions). The structure, colored by block boundaries, was generated using the Structure Viewer tool at: alphafold.ebi.ac.uk/entry/X5DQM5.

Testing Whether Deletions Occur More Often in Regions with Long Repeats

To assess whether deletions are more likely to occur in regions with repeated sequences, the 101 bp window surrounding the start and end positions of identified deletions was locally aligned to each other using BLAST (v2.16.0). If the same deletion was detected multiple times across different read length clusters within an assembly replicate, the deletion was only considered once. A total of 117 deletions total were examined. The BLAST “blastn” function was employed with the following parameters: -word_size 4 -reward 1 -penalty -1 -gapopen 5 -gapextend 2. For each deletion, 100 random pairs of 101 bp windows, separated by the same distance as the deletion, were randomly chosen from the construct where the deletion was detected. Each random pair of windows was also locally aligned to each other with blastn using the same parameters. A one-sided Wilcoxon rank-sum test was then used to compare the max BLAST scores with those of the randomly selected regions to test for significant differences.

### Construction of Bridge Donor Plasmids

Bridge sequences that are 80-140 bp long were used to stitch together DNA blocks without homology. To scarlessly stitch full-length DNA blocks together, the first 40-70 bp of the bridge sequence was designed to match the last 40-70 bp of the preceding DNA block, and the second 40-70 bp was designed to match the first 40-70 bp of the next incoming DNA block. To stitch together parts of existing DNA blocks, the first 40-70 bp of the bridge sequence was designed to match the last 40-70 bp of the desired region in the preceding DNA block, and the second 40-70 bp was designed to match the first 40-70 bp of the desired region in the next incoming DNA block.

To construct the bridge donor plasmids, three different sets of bridge sequences, each flanked by AscI and NotI restriction sites, were designed on the same DNA fragment and ordered from TWIST. The TWIST DNA fragment was then digested using AscI and NotI restriction enzymes, and fragments of the expected size were gel extracted. The extracted gel fragment was ligated with AscI and NotI digested odd or even donor plasmids using T4 ligase and transformed into the dSL2 donor strain. Cells carrying the correct bridge donor plasmid were selected on LB+100 μg/mL Hyg+2 μg/mL Tc agar plates for odd donor plasmids, and with LB+60 μg/mL Sp+2 μg/mL Tc agar plates for even donor plasmids. The identity of the donor plasmids were then determined by Sanger sequence using primer oSL8.

For stitching together two DNA blocks where one is in an odd donor plasmid or the other is in an even donor plasmid, only one stitch with a donor plasmid containing the bridge sequence is necessary (e.g., odd—even bridge—odd). However, for stitching together pairs of DNA blocks where both are in odd donor plasmids or even donor plasmids, two stitching steps are required to ensure that the selection and counter-selection markers are in the correct orientation (e.g., odd—even bridge 1—odd bridge 2—even). In these cases, the bridge sequence is the same for both odd and even bridge donor plasmids. Because the bridge sequence remains the same, the assembled product on the recipient remains the same; only the downstream selection and counter-selection cassettes are exchanged.

### Transfer of Assembled Products to Donor Plasmids

To transfer assembled products from recipient plasmids to donor plasmids, the recipient plasmids were digested with AscI and NotI restriction enzymes, and DNA fragments of the expected size were gel extracted. The gel extracted fragments were ligated with AscI and NotI digested donor plasmids using T4 ligase, and transformed into the dSL2 donor strain. Cells carrying the correct donor plasmids were selected on LB+100 μg/mL Hyg+2 μg/mL Tc agar plates.

### REFERENCES

1. Gibson, D. G. et al. Enzymatic assembly of DNA molecules up to several hundred kilobases. Nat. Methods 6, 343-345 (2009).
2. Engler, C., Gruetzner, R., Kandzia, R. & Marillonnet, S. Golden Gate Shuffling: A One-Pot DNA Shuffling Method Based on Type IIs Restriction Enzymes. PLOS ONE 4, e5553 (2009).
3. Weber, E., Engler, C., Gruetzner, R., Werner, S. & Marillonnet, S. A Modular Cloning System for Standardized Assembly of Multigene Constructs. PLOS ONE 6, e16765 (2011).
4. Hillson, N. et al. Building a global alliance of biofoundries. Nat. Commun. 10, 2040 (2019).
5. Ma, Y., Zhang, Z., Jia, B. & Yuan, Y. Automated high-throughput DNA synthesis and assembly. Heliyon 10, e26967 (2024).
6. Li, M. Z. & Elledge, S. J. MAGIC, an in vivo genetic method for the rapid construction of recombinant DNA molecules. Nat. Genet. 37, 311-319 (2005).
7. Wang, K. et al. Defining synonymous codon compression schemes by genome recoding. Nature 539, 59-64 (2016).
8. Zürcher, J. F. et al. Continuous synthesis of E. coli genome sections and Mb-scale human DNA assembly. Nature 1-8 (2023) doi:10.1038/s41586-023-06268-1.
9. Jinek, M. et al. A programmable dual-RNA-guided DNA endonuclease in adaptive bacterial immunity. Science 337, 816-821 (2012).
10. Jiang, W., Bikard, D., Cox, D., Zhang, F. & Marraffini, L. A. RNA-guided editing of bacterial genomes using CRISPR-Cas systems. Nat. Biotechnol. 31, 233-239 (2013).
11. Thomason, L. C., Sawitzke, J. A., Li, X., Costantino, N. & Court, D. L. Recombineering: genetic engineering in bacteria using homologous recombination. Curr. Protoc. Mol. Biol. 106, 1.16.1-1.16.39 (2014).
12. Hoi, H. et al. An Engineered Monomeric Zoanthus sp. Yellow Fluorescent Protein. Chem. Biol. 20, 1296-1304 (2013).
13. Li, W. et al. Arrayed in vivo barcoding for multiplexed sequence verification of plasmid DNA and demultiplexing of pooled libraries. Nucleic Acids Res. 52, e47 (2024).
14. Oberortner, E., Cheng, J.-F., Hillson, N. J. & Deutsch, S. Streamlining the Design-to-Build Transition with Build-Optimization Software Tools. ACS Synth. Biol. 6, 485-496 (2017).
15. Chayot, R., Montagne, B., Mazel, D. & Ricchetti, M. An end-joining repair mechanism in Escherichia coli. Proc. Natd. Acad. Sci. 107, 2141-2146 (2010).
16. Levy, S. F. et al. Quantitative evolutionary dynamics using high-resolution lineage tracking. Nature 519, 181-186 (2015).
17. Bosch, J. From software product lines to software ecosystems. in Proceedings of the 13th International Software Product Line Conference 111-119 (Carnegie Mellon University, USA, 2009).
18. Oster, C. & Wade, J. Ecosystem requirements for composability and reuse: An investigation into ecosystem factors that support adoption of composable practices for engineering design. Syst. Eng. 16, 439-452 (2013).
19. Shetty, R. P., Endy, D. & Knight, T. F. Engineering BioBrick vectors from BioBrick parts. J. Biol. Eng. 2, 5 (2008).
20. Pindy-Szekeres, G. et al. GPCRdb in 2023: state-specific structure models using AlphaFold2 and new ligand resources. Nucleic Acids Res. 51, D395-D402 (2023).
21. Boyer, H. W. & Roulland-dussoix, D. A complementation analysis of the restriction and modification of DNA in Escherichia coli. J. Mol. Biol. 41, 459-472 (1969).
22. Jensen, S. I., Lennen, R. M., Herrgird, M. J. & Nielsen, A. T. Seven gene deletions in seven days: Fast generation of Escherichia coli strains tolerant to acetate and osmotic stress. Sci. Rep. 5, 17874 (2015).
23. Rakowski, S. A. & Filutowicz, M. Plasmid R6K Replication Control. Plasmid 69, 231-242 (2013).
24. Miyazaki, K. Molecular Engineering of a PheS Counterselection Marker for Improved Operating Efficiency in Escherichia Coli. BioTechniques 58, 86-88 (2015).
25. Garcia-Nafria, J., Watson, J. F. & Greger, I. H. IVA cloning: A single-tube universal cloning system exploiting bacterial In Vivo Assembly. Sci. Rep. 6, 27459 (2016).
26. Lengger, B. & Jensen, M. K. Engineering G protein-coupled receptor signalling in yeast for biotechnological and medical purposes. FEMS Yeast Res. 20, foz087 (2020).
27. Kapolka, N. J. et al. DCyFIR: a high-throughput CRISPR platform for multiplexed G protein-coupled receptor profiling and ligand discovery. Proc. Natl. Acad. Sci. 117, 13117-13126 (2020).
28. Li, H. Minimap2: pairwise alignment for nucleotide sequences. Bioinformatics 34, 3094-3100 (2018).
29. Streck, A., Kaufmann, T. L. & Schwarz, R. F. SMITH: spatially constrained stochastic model for simulation of intra-tumour heterogeneity. Bioinformatics 39, btad102 (2023).
30. Li, H. New strategies to improve minimap2 alignment accuracy. Bioinformatics 37, 4572-4574 (2021).
31. Danecek, P. et al. Twelve years of SAMtools and BCFtools. GigaScience 10, giab008 (2021).
32. Smolka, M. et al. Detection of mosaic and population-level structural variants with Sniffles2. Nat. Biotechnol. 1-10 (2024) doi:10.1038/s41587-023-02024-y.
33. Katoh, K. & Standley, D. M. MAFFT Multiple Sequence Alignment Software Version 7: Improvements in Performance and Usability. Mol. Biol. Evol. 30, 772-780 (2013).

### Sequences:

| SEQ ID NO | Description |
|---|---|
| 1 | eBSD16_Hyg_pLann |
| 2 | eBSD28_Recp_help |
| 3 | eaBSD35_recipient plasmid |
| 4 | eBSD13_1st_odd_donor plasmid |
| 5 | eBSD19_Sp_pLann_1st odd donor |
| | plasmid |
| 6 | eBSD22_Apm_PheS_even donor |
| | plasmid |
| 7 | SacB |
| 8 | HygR |
| 9 | Replication initiation protein |
| 10 | spCas9 |
| 11 | AraC |
| 12 | Host nuclease inhibitor Gam |
| 13 | Phage recombination protein Bet |
| 14 | Lambda exonuclease |
| 15 | Beta lactamase (Amp resistance) |
| 16 | Phe t-RNA synthetase subunit alpha |
| 17 | Gentamycin resistance |
| 18 | Streptomycin resistance |
| 19 | Apramycin resistance |

What is claimed is:

1. A method of truncating and, optionally, replacing, at least one DNA sequence, the method comprising:
(a) conjugating a first donor cell comprising a first donor construct with a first recipient cell comprising a first recipient polynucleotide to (i) transfer the first donor construct from the first donor cell to the first recipient cell and (ii) recombine the first donor construct and the first recipient construct in the first recipient cell by homologous recombination, wherein:
the first donor construct comprises, from 5′ to 3′, (1) a first endonuclease site (C1), (2) a first homologous recombination region (HR1), optionally, a first joining polynucleotide sequence, which optionally comprises a seventh homologous recombination region (HR7), (3) a first selection cassette comprising at least one first selectable marker, (4) a second homologous recombination region (HR2), and (5) a second endonuclease site (C2);
the recipient construct comprises, from 5′ to 3′, (1) a first recipient polynucleotide comprising a first sequence to be truncated comprising a third homologous recombination region (HR3) that is homologous to HR1, and (2) a fourth homologous recombination region (HR4) that is homologous to HR2, and, optionally, further comprising a removable selectable construct comprising from 5′ to 3′ a third endonuclease site (C3), a second selection cassette comprising at least one second selectable marker, wherein the at least one second selectable marker is distinct from the at least one first selectable marker in the first selection cassette, and a fourth endonuclease site (C4), wherein HR3 ends at least one base pair (bp) upstream of the 3′ end of the first sequence to be truncated;
wherein, following the conjugation of the first donor cell and the first recipient cell, the first recipient cell comprises one or more endonuclease specific for C1, C2, and optionally C3, and C4, such that C1, C2, and optionally C3, and C4 are cleaved;
thereby providing, following the homologous recombination of HR1 with HR3 and HR2 with HR4, a first recombined polynucleotide in the recipient cell comprising, from 5′ to 3′, a fragment of the first sequence to be truncated, HR1/HR3, optionally, the first joining polynucleotide sequence, the first selection cassette, and HR2/HR4.

2. The method of claim 1, further comprising selecting for the first selectable marker after recombination of HR1 with HR3 and HR2 with HR4 to select for the first recombined polynucleotide.

3. The method of claim 1, wherein the first donor construct comprises, from 5′ to 3′, (1) the first endonuclease site (C1), (2) the first homologous recombination region (HR1), optionally, the first joining polynucleotide sequence, which optionally comprises a seventh homologous recombination region (HR7), a fifth endonuclease site (C5), the first selection cassette comprising at least one selectable marker, a sixth endonuclease site (C6), the second homologous recombination region (HR2), and (5) the second endonuclease site (C2).

4. The method of claim 1, wherein the first donor construct comprises a fifth endonuclease (C5) and a sixth endonuclease site (C6) flanking the first selection cassette.

5. The method of claim 4, further comprising:
(b) conjugating a second donor cell comprising a second donor construct to the cell comprising the first recombined polynucleotide to (i) transfer the second donor construct from the second donor cell to the cell comprising the first recombined polynucleotide and (ii) recombine the second donor construct and the first recombined polynucleotide in the recipient cell by homologous recombination, wherein:
the second donor construct comprises, from 5′ to 3′, (1) a seventh endonuclease site (C7), (2) a fifth homologous recombination region (HR5) that is homologous to HR7, optionally, a second joining polynucleotide sequence, (3) an optional ninth endonuclease site (C9), (4) a third selection cassette comprising at least one selectable marker, wherein the at least one selectable marker in the third selection cassette is distinct from the at least one first selectable marker in the first selection cassette, (5) an optional tenth endonuclease site (C10), (6) a sixth homologous recombination region (HR6) that is homologous to HR2/HR4, and (7) an eighth endonuclease site (C8);
the first recombined polynucleotide in the recipient cell comprises, from 5′ to 3′, a fragment of the first sequence to be truncated, HR1/HR3, the first joining polynucleotide sequence comprising HR7, the first selection cassette optionally with C5 upstream or 5′ of the selection cassette and C6 downstream or 3′ of the selection cassette, and HR2/HR4;
wherein the cell comprising the first recombined polynucleotide comprises at least one endonuclease specific for C7 and C8, such that are C7 and C8 are cleaved;
thereby providing, following the homologous recombination of HR5 and HR7 and also HR6 and HR2/HR4, a second recombined polynucleotide, the second recombined polynucleotide comprising, from 5′ to 3′, a fragment of the first sequence to be truncated, HR1/HR3, optionally, the first joining polynucleotide sequence or portion thereof 5′ or upstream of HR7, HR5/HR7, optionally, the second joining polynucleotide sequence, optionally C9, the third selection cassette, optionally C10, and HR2/HR4/HR6.

6. The method of claim 1, wherein
the first donor construct comprises, from 5′ to 3′, HR1, the first joining sequence comprising the seventh homologous recombination region (HR7), thereby generating the first recombined polynucleotide comprising, from 5′ to 3′, a fragment of the first sequence to be truncated, HR1, the first joining polynucleotide sequence, the first selection cassette, and HR2/HR4, wherein the first selection cassette is optionally flanked by endonuclease sites.

7. The method of claim 1, wherein the removable selectable construct is located between HR3 and HR4.

8. The method of claim 1, wherein
(i) the first donor cell comprises a polynucleotide encoding one or more homologous DNA repair genes that is transferred to the first recipient cell by conjugation;
(ii) the first recipient cell comprises a polynucleotide encoding one or more homologous DNA repair genes on a self-replicating construct; or
(iii) the first recipient cell comprises a polynucleotide encoding one or more homologous DNA repair genes that is integrated into a genome of the first recipient cell.

9. The method of claim 8, wherein the one or more homologous DNA repair genes comprise the lambda red homologous repair genes.

10. The method of claim 1, wherein the one or more endonuclease comprises an RNA-guided DNA endonuclease and wherein
(i) the first donor cell comprises a polynucleotide encoding one or more guide RNAs (gRNAs) that is transferred to the first recipient cell by conjugation;
(ii) the first recipient cell comprises a polynucleotide encoding one or more guide RNAs (gRNAs) on a self-replicating construct; or
(iii) the first recipient cell comprises a polynucleotide encoding one or more guide RNAs (gRNAs) that is integrated into a genome of the first recipient cell.

11. The method of claim 10, wherein the one or more guide RNAs each bind to one endonuclease site.

12. The method of claim 10, wherein
(i) the first donor cell comprises a polynucleotide encoding the RNA-guided DNA endonuclease that is transferred to the first recipient cell by conjugation;
(ii) the first recipient cell comprises a polynucleotide encoding the RNA-guided DNA endonuclease on a self-replicating construct; or
(iii) the first recipient cell comprises a polynucleotide encoding the RNA-guided DNA endonuclease that is integrated into a genome of the first recipient cell.

13. The method of claim 12, wherein the RNA-guided endonuclease is selected from the group consisting of Cas1, Cas1B, Cas2, Cas3, Cas4, Cas5, Cas6, Cas7, Cas8, Cas9, Cas10, Cas12, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

14. The method of claim 1, wherein the at least one selectable marker comprises a positive-selectable marker and/or a counter-selectable marker and the method further comprises selecting recipient cells based on the presence of the positive-selectable marker and/or the absence of the counter-selectable marker.

15. The method of claim 1, wherein the donor construct comprises a conditional origin of replication, wherein replication of the donor construct is dependent on a conditional replication factor, and wherein the first recipient cell does not comprise the conditional replication factor.

16. The method of claim 4, wherein step (b) is repeated for one or more iterations with a third or subsequent donor cell comprising a third or subsequent donor construct comprising compatible HR regions and a third or subsequent donor polynucleotide, thereby forming a third or subsequent recombined polynucleotide.

17. The method of claim 1, wherein expression of the one or more endonucleases is inducible and the method further comprises inducing expression of the one or more endonucleases.

18. A method of truncating and, optionally, replacing, at least one DNA sequence, the method comprising:
(a) conjugating a first donor cell with a first recipient cell, wherein the first donor cell comprises a first donor construct and wherein the first recipient cell comprises a first recipient polynucleotide, to (i) transfer the first donor construct from the first donor cell to the first recipient cell and (ii) recombine the first donor construct and the first recipient construct in the first recipient cell by homologous recombination, wherein:
the first donor construct comprises, from 5′ to 3′, (1) a first endonuclease site (C11), a first sequence to be truncated comprising a first homologous recombination region (HR1), (2) a first selection cassette comprising at least one first selectable marker, (3) a second homologous recombination region (HR2), and (4) a second endonuclease site (C2), wherein HR1 is located at least one bp downstream of the 5′ end of the first sequence to be truncated and wherein the first selection cassette is optionally flanked by endonuclease sites, such that a fifth endonuclease site (C5) is positioned 5′ of the first selection cassette and a sixth endonuclease site (C6) is positioned 3′ of the first selection cassette when present;
the recipient construct comprises, from 5′ to 3′, optionally, a first joining sequence, (1) a third homologous recombination region (HR3) that is homologous to HR1, and (2) a fourth homologous recombination region (HR4) that is homologous to HR2, and optionally further comprising a removable selectable construct positioned between HR3 and HR4 comprising from 5′ to 3′ an optional third endonuclease site (C3), a second selection cassette comprising at least one second selectable marker, wherein the at least one second selectable marker is distinct from the at least one first selectable marker in the first selection cassette, and a fourth endonuclease site (C4);
wherein, following the conjugation of the first donor cell and the first recipient cell, the first recipient cell comprises one or more endonuclease specific for C11, C2, and optionally C3, and C4, such that C11, C2, and optionally C3, and C4 are cleaved;
thereby providing, following the homologous recombination of HR1 with HR3 and HR2 with HR4, a first recombined polynucleotide in the recipient cell comprising, from 5′ to 3′, optionally, the first joining sequence, HR1/HR3, a 3′ fragment of the first sequence to be truncated, the first selection cassette, and HR2/HR4.

19. A method of joining two polynucleotide sequences, the method comprising:
(a) conjugating a first donor cell comprising a first bridging construct with a first recipient cell comprising a first recipient polynucleotide to (i) transfer the first bridging construct from the first donor cell to the first recipient cell and (ii) recombine the first bridging construct and the first recipient construct in the first recipient cell by homologous recombination, wherein:
the first bridging construct comprises, from 5′ to 3′, (1) a first endonuclease site (C1), (2) a first homologous recombination region (HR1), (3) a fifth homologous recombination region (HR5), (4) an optional fifth endonuclease site (C5), (5) a first selection cassette comprising at least one first selectable marker, (6) an optional sixth endonuclease site (C6), (7) a second homologous recombination region (HR2), and (8) a second endonuclease site (C2), the first bridging construct may also optionally contain a first sequence to be joined in the bridging construct between HR1 and HR5;
the recipient construct comprises, from 5′ to 3′, an optional first joining polynucleotide comprising a third homologous recombination region (HR3); a fourth homologous recombination region (HR4); and optionally further comprising, between HR3 and HR4, a removable selectable construct comprising an optional third endonuclease site (C3), a second selection cassette comprising at least one second selectable marker, wherein the at least one second selectable marker is distinct from the at least one first selectable marker in the first selection cassette, and an optional fourth endonuclease site (C4);
wherein, following the conjugation of the first donor cell and the first recipient cell, the first recipient cell comprises one or more endonucleases specific for C1 and C2, and optionally C3 and C4, such that C1 and C2, and optionally C3 and C4 are cleaved;
thereby providing, following the homologous recombination of HR1 with HR3 and HR2 with HR4, a first recombined polynucleotide in the recipient cell comprising, from 5′ to 3′, the portion of the first joining polynucleotide upstream of HR3, HR1/HR3, optionally the first sequence to be joined, HR5, optionally C5, the first selection cassette, optionally C6, and HR2/HR4;
(b) conjugating a second donor cell comprising a second donor construct to the cell comprising the first recombined polynucleotide to (i) transfer the second donor construct from the second donor cell to the cell comprising the first recombined polynucleotide and (ii) recombine the second donor construct and the first recombined polynucleotide in the recipient cell by homologous recombination, wherein:
the first donor construct comprises, from 5′ to 3′, (1) a seventh endonuclease site (C7), (2) a second joining polynucleotide sequence comprising a sixth homologous recombination region (HR6) that is homologous to HR5, (3) an optional ninth endonuclease site (C9), (4) a third selection cassette comprising at least one selectable marker, wherein the at least one selectable marker is distinct from the at least one first selectable marker in the first selection cassette, (5) an optional tenth endonuclease site (C10), (6) a seventh homologous recombination region (HR7), and (7) an eighth endonuclease site (C8);
wherein the cell comprising the first recombined polynucleotide comprises at least one endonuclease specific for C7 and C8, such that are C7 and C8 are cleaved;
thereby providing, following the homologous recombination of HR6 and HR5 and HR7 and HR2, a second recombined polynucleotide comprising, from 5′ to 3′: the portion of the first joining polynucleotide upstream of HR3, HR3, HR5/HR6, the second joining polynucleotide, optionally C9, the third selection cassette, optionally C10, and HR2/HR7.

20. The method of claim 19, wherein (I) HR3 begins at least one base pair (bp) downstream of the 5′ end of the first joining polynucleotide and/or (II) HR5 begins at least one bp downstream from the 5′ end of the second joining polynucleotide.
